SEQUENCE LISTING (34 sequences)

SEQ ID NO: 1 - protein, 8 aa, Mus musculus
  misc_feature (1)..(8): anti-hAPRIL heavy chain CDR1 (AA)
GYTFTSYV

SEQ ID NO: 2 - protein, 16 aa, Mus musculus
  misc_feature (1)..(16): anti-hAPRIL heavy chain CDR2 (AA)
YINPYNDAPKYNEKFK

SEQ ID NO: 3 - protein, 12 aa, Mus musculus
  misc_feature (1)..(12): anti-hAPRIL heavy chain CDR3 (AA)
GLGYALYYAMDY

SEQ ID NO: 4 - protein, 11 aa, Mus musculus
  misc_feature (1)..(11): anti-hAPRIL light chain CDR1 (AA)
KASQNVGNNVA

SEQ ID NO: 5 - protein, 7 aa, Mus musculus
  misc_feature (1)..(7): anti-hAPRIL light chain CDR2 (AA)
SASNRDS

SEQ ID NO: 6 - protein, 10 aa, Mus musculus
  misc_feature (1)..(10): anti-hAPRIL light chain CDR3 (AA)
QQYNIYPFTF

SEQ ID NO: 7 - DNA, 363 bp, Artificial Sequence
  Synthetic VH11 heavy chain variable region (DNA)
caggtccagcttgtgcagtctggggctgaggtgaagaagcctggggcctcagtgaaggtttcctgcaaggcttctggatacacattcactagctatgtgatgcattgggtgcgccaggcccccggacaaaggcttgagtggatgggatatattaatccttataatgatgctcctaaatacaatgagaagttcaaaggcagagtcaccattaccagggacacatccgcgagcacagcctacatggagctgagcagcctgagatctgaagacacggctgtgtattactgtgcgagaggcttgggttacgccctttactatgctatggactactggggccaagggaccacggtcaccgtctcctca

SEQ ID NO: 8 - protein, 121 aa, Artificial Sequence
  Synthetic VH11 heavy chain variable region (AA)
QVQLVQSGAEVKKPGASVKVSCKASGYTFTSYVMHWVRQAPGQRLEWMGYINPYNDAPKYNEKFKGRVTITRDTSASTAYMELSSLRSEDTAVYYCARGLGYALYYAMDYWGQGTTVTVSS

SEQ ID NO: 9 - DNA, 363 bp, Artificial Sequence
  Synthetic VH12 heavy chain variable region (DNA)
caggtccagcttgtgcagtctggggctgaggtgaagaagcctggggcctcagtgaaggtctcctgcaaggcttctggatacacattcactagctatgtgatgcactgggtgcgacaggcccctggacaagggcttgagtggatgggatatattaatccttataatgatgctcctaaatacaatgagaagttcaaaggcagggtcaccatgaccagggacacgtccatcagcacagcctacatggagctgagcaggctgagatctgacgacacggccgtgtattactgtgcgagaggcttgggttacgccctttactatgctatggactactggggccaagggaccacggtcaccgtctcgagc

SEQ ID NO: 10 - protein, 121 aa, Artificial Sequence
  Synthetic VH12 heavy chain variable region (AA)
QVQLVQSGAEVKKPGASVKVSCKASGYTFTSYVMHWVRQAPGQGLEWMGYINPYNDAPKYNEKFKGRVTMTRDTSISTAYMELSRLRSDDTAVYYCARGLGYALYYAMDYWGQGTTVTVSS

SEQ ID NO: 11 - DNA, 363 bp, Artificial Sequence
  Synthetic VH13 heavy chain variable region (DNA)
caggtccagcttgtgcagtctggggctgaggtgaagaagcctggggcctcagtgaaggtttcctgcaaggcatctggatacacattcactagctatgtgatgcactgggtgcgacaggcccctggacaagggcttgagtggatgggatatattaatccttataatgatgctcctaaatacaatgagaagttcaaaggcagagtcaccatgaccagggacacgtccacgagcacagtctacatggagctgagcagcctgagatctgaggacacggccgtgtattactgtgcgagaggcttgggttacgccctttactatgctatggactactggggccaagggacaatggtcaccgtctcgagc

SEQ ID NO: 12 - protein, 121 aa, Artificial Sequence
  Synthetic VH13 heavy chain variable region (AA)
QVQLVQSGAEVKKPGASVKVSCKASGYTFTSYVMHWVRQAPGQGLEWMGYINPYNDAPKYNEKFKGRVTMTRDTSTSTVYMELSSLRSEDTAVYYCARGLGYALYYAMDYWGQGTMVTVSS

SEQ ID NO: 13 - DNA, 363 bp, Artificial Sequence
  Synthetic VH14 heavy chain variable region (DNA)
caggtccagcttgtgcagtctggggctgaggtgaagaagcccggcgccagcgtgaaggtgagctgcaaggccagcggatacacattcactagctatgtgatgcactgggtgagacaggcccccggccagggcctggagtggatgggctatattaatccttataatgatgctcctaaatacaatgagaagttcaaaggcagagtgaccatgaccagagacaccagcgccagcaccgcctacatggagctgagcagcctgagaagcgacgacaccgccgtgtactactgcgccagaggcttgggttacgccctttactatgctatggactactggggccagggcaccaccgtgaccgtgagcagc

SEQ ID NO: 14 - protein, 121 aa, Artificial Sequence
  Synthetic VH14 heavy chain variable region (AA)
QVQLVQSGAEVKKPGASVKVSCKASGYTFTSYVMHWVRQAPGQGLEWMGYINPYNDAPKYNEKFKGRVTMTRDTSASTAYMELSSLRSDDTAVYYCARGLGYALYYAMDYWGQGTTVTVSS

SEQ ID NO: 15 - DNA, 363 bp, Artificial Sequence
  Synthetic VH14_1 heavy chain variable region (DNA)
caggtccagcttgtgcagtctggggctgaggtgaagaagcccggcgccagcgtgaaggtgagctgcaaggccagcggatacacattcactagctatgtgatgcactgggtgagacaggcccccggccagggcctggagtggatgggctatattaatccttataatgatgctcctaaatacaatgagaagttcaaaggcagagtgaccatgaccagtgacaccagcgccagcaccgcctacatggagctgagcagcctgagaagcgacgacaccgccgtgtactactgcgccagaggcttgggttacgccctttactatgctatggactactggggccagggcaccaccgtgaccgtgagcagc

SEQ ID NO: 16 - protein, 121 aa, Artificial Sequence
  Synthetic VH14_1 heavy chain variable region (AA)
QVQLVQSGAEVKKPGASVKVSCKASGYTFTSYVMHWVRQAPGQGLEWMGYINPYNDAPKYNEKFKGRVTMTSDTSASTAYMELSSLRSDDTAVYYCARGLGYALYYAMDYWGQGTTVTVSS

SEQ ID NO: 17 - DNA, 363 bp, Artificial Sequence
  Synthetic VH14_1C heavy chain variable region (DNA)
caggtccagcttgtgcagtctggggctgaggtgaagaagcccggcgccagcgtgaaggtgagctgcaaggccagcggatacacattcactagctatgtgatgcactgggtgagacaggcccccggccagggcctggagtggatgggctatattaatccttataatgatgctcctaaatacaatgagaagttcaaaggcagagtgaccatcaccagtgacaccagcgccagcaccgcctacatggagctgagcagcctgagaagcgacgacaccgccgtgtactactgcgccagaggcttgggttacgccctttactatgctatggactactggggccagggcaccaccgtgaccgtgagcagc

SEQ ID NO: 18 - protein, 121 aa, Artificial Sequence
  Synthetic VH14_1C heavy chain variable region (AA)
QVQLVQSGAEVKKPGASVKVSCKASGYTFTSYVMHWVRQAPGQGLEWMGYINPYNDAPKYNEKFKGRVTITSDTSASTAYMELSSLRSDDTAVYYCARGLGYALYYAMDYWGQGTTVTVSS

SEQ ID NO: 19 - DNA, 363 bp, Artificial Sequence
  Synthetic VH14_1D heavy chain variable region (DNA)
caggtccagcttgtgcagtctggggctgaggtgaagaagcccggcgccagcgtgaaggtgagctgcaaggccagcggatacacattcactagctatgtgatgcactgggtgagacaggcccccggccagggcctggagtggatgggctatattaatccttataatgatgctcctaaatacaatgagaagttcaaaggcagagtgaccatgaccagtgacaagagcgccagcaccgcctacatggagctgagcagcctgagaagcgacgacaccgccgtgtactactgcgccagaggcttgggttacgccctttactatgctatggactactggggccagggcaccaccgtgaccgtgagcagc

SEQ ID NO: 20 - protein, 121 aa, Artificial Sequence
  Synthetic VH14_1D heavy chain variable region (AA)
QVQLVQSGAEVKKPGASVKVSCKASGYTFTSYVMHWVRQAPGQGLEWMGYINPYNDAPKYNEKFKGRVTMTSDKSASTAYMELSSLRSDDTAVYYCARGLGYALYYAMDYWGQGTTVTVSS

SEQ ID NO: 21 - DNA, 363 bp, Artificial Sequence
  Synthetic VH14_1E heavy chain variable region (DNA)
gaggtccagcttgtgcagtctggggctgaggtgaagaagcccggcgccagcgtgaaggtgagctgcaaggccagcggatacacattcactagctatgtgatgcactgggtgagacaggcccccggccagggcctggagtggatgggctatattaatccttataatgatgctcctaaatacaatgagaagttcaaaggcagagtgaccatgaccagtgacaccagcgccagcaccgcctacatggagctgagcagcctgagaagcgacgacaccgccgtgtactactgcgccagaggcttgggttacgccctttactatgctatggactactggggccagggcaccaccgtgaccgtgagcagc

SEQ ID NO: 22 - protein, 121 aa, Artificial Sequence
  Synthetic VH14_1E heavy chain variable region (AA)
EVQLVQSGAEVKKPGASVKVSCKASGYTFTSYVMHWVRQAPGQGLEWMGYINPYNDAPKYNEKFKGRVTMTSDTSASTAYMELSSLRSDDTAVYYCARGLGYALYYAMDYWGQGTTVTVSS

SEQ ID NO: 23 - DNA, 363 bp, Artificial Sequence
  Synthetic VH14_1G heavy chain variable region (DNA)
caggtccagcttgtgcagtctggggctgaggtgaagaagcccggcgccagcgtgaaggtgagctgcaaggccagcggatacacattcactagctatgtgatgcactgggtgagacaggcccccggccagggcctggagtggatgggctatattaatccttataatgatgctcctaaatacaatgagaagttcaaaggcaaagcgaccatgaccagtgacaccagcgccagcaccgcctacatggagctgagcagcctgagaagcgacgacaccgccgtgtactactgcgccagaggcttgggttacgccctttactatgctatggactactggggccagggcaccaccgtgaccgtgagcagc

SEQ ID NO: 24 - protein, 121 aa, Artificial Sequence
  Synthetic VH14_1G heavy chain variable region (AA)
QVQLVQSGAEVKKPGASVKVSCKASGYTFTSYVMHWVRQAPGQGLEWMGYINPYNDAPKYNEKFKGKATMTSDTSASTAYMELSSLRSDDTAVYYCARGLGYALYYAMDYWGQGTTVTVSS

SEQ ID NO: 25 - DNA, 321 bp, Artificial Sequence
  Synthetic VL15 light chain variable region (DNA)
gacatcgtgatgacccagtctccttccaccctgtctgcatctgtaggagacagagtcaccatcacttgcaaggccagtcagaatgtgggtaataatgtagcctggtatcagcagaaaccagggaaagcccctaagctcctgatctcttcggcatccaaccgtgacagtggggtcccatcaaggttcagcggcagtggatctgggacagagttcactctcaccatcagcagcctgcagcctgatgattttgcaacttatttctgccagcaatataacatctatccattcacgtttggccaggggaccaagctggagatcaaa

SEQ ID NO: 26 - protein, 107 aa, Artificial Sequence
  Synthetic VL15 light chain variable region (AA)
DIVMTQSPSTLSASVGDRVTITCKASQNVGNNVAWYQQKPGKAPKLLISSASNRDSGVPSRFSGSGSGTEFTLTISSLQPDDFATYFCQQYNIYPFTFGQGTKLEIK

SEQ ID NO: 27 - DNA, 1344 bp, Artificial Sequence
  Synthetic VH14_1G heavy chain (DNA)
caggtccaacttgtgcagtctggggctgaagtgaagaagcccggcgctagtgtgaaggtttcatgtaaggcttctggatacacatttacttcatatgtaatgcactgggtgcgtcaagcccctggccagggcctggagtggatggggtatattaatccttataatgatgctcctaaatacaatgagaagttcaaaggaaaagcaactatgaccagtgatactagcgcttcaaccgcctacatggagctgagcagcttaagaagcgacgacaccgccgtgtactattgtgccaggggcttgggttacgccctttattatgctatggactactggggtcagggcaccacagtgaccgttagctctgcatctactaagggaccatccgtcttccccctggcgccatgctcccgcagtacaagtgagagcacagcagccctgggctgtttggtaaaggactacttccccgaacctgtgactgtgtcttggaactcaggcgccctgactagcggcgtgcacactttccctgctgtcctacagtcctcaggactatactccctctcgtctgtggtgacagtgccttcctcatcattgggaacgaaaacctatacttgcaacgttgatcacaagcccagcaacaccaaggtggacaagagagttgagtccaaatatggtcccccatgtccaccatgtccagcacctgagtttcttggcggaccaagtgttttcctgttccccccaaaacccaaggatactctcatgataagtcgcacccctgaagtcacttgcgtggtggtggacgttagccaggaagatcccgaagtccaattcaactggtacgtagatggcgtagaagtgcataatgcgaagacaaagccgagagaggagcagtttaattcgacgtatcgggtggtcagcgtcctcacagtcctgcaccaggactggctgaacggcaaggagtataagtgcaaggtctccaacaaaggtctcccgtcctccattgagaaaacaatctccaaagcaaaagggcagccccgagaaccacaagtgtacaccctgcccccatctcaggaggagatgaccaagaaccaggtcagtcttacctgcctggtcaaaggcttttatccctcagatatcgccgttgagtgggaaagcaatgggcagccggagaacaactacaagaccacgcctcccgttctggattctgacggatcgttctttttatacagcaggctaaccgtggacaagtctcggtggcaggaagggaatgtattttcttgcagtgtaatgcatgaggctctgcacaatcattacacacagaagtctctctccctgtctcttggtaaa

SEQ ID NO: 28 - protein, 448 aa, Artificial Sequence
  Synthetic VH14_1G heavy chain (AA)
QVQLVQSGAEVKKPGASVKVSCKASGYTFTSYVMHWVRQAPGQGLEWMGYINPYNDAPKYNEKFKGKATMTSDTSASTAYMELSSLRSDDTAVYYCARGLGYALYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 29 - DNA, 642 bp, Artificial Sequence
  Synthetic VL15 light chain (DNA)
gacattgtgatgacccagtctccttccactctctctgcatctgtgggagacagagtaaccatcacttgcaaggccagtcagaatgtgggtaataatgtagcctggtatcagcagaaaccagggaaagcccctaagctactgatctcttccgcatccaaccgggacagtggtgtgccctcaaggtttagcggcagtggatcagggacagagttcacattgaccatatccagcctgcagcctgatgattttgctacttatttctgccaacaatataacatttacccattcacgtttggccagggcaccaagctagagatcaaacggacggttgctgcaccctctgtctttatcttcccgccatctgatgaacagttgaagtccggaacagcctctgttgtgtgcctgctgaataacttttatccccgcgaggcgaaagttcagtggaaggtggataacgccctccaatcaggcaattcccaggagagtgtgacagagcaagattccaaggactcaacctacagcctcagcagtactttaactctgagcaaagcagactacgagaagcacaaagtctacgcttgcgaagtcacccatcagggccttagctcgcccgtcacaaagagctttaacaggggagaatgt

SEQ ID NO: 30 - protein, 214 aa, Artificial Sequence
  Synthetic VL15 light chain (AA)
DIVMTQSPSTLSASVGDRVTITCKASQNVGNNVAWYQQKPGKAPKLLISSASNRDSGVPSRFSGSGSGTEFTLTISSLQPDDFATYFCQQYNIYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - DNA, 57 bp, Homo sapiens
  misc_feature (1)..(57): Heavy chain secretion leader sequence (DNA)
atggactggacctggaggatcctctttttggtggcagcagccacaggtgcccactcc

SEQ ID NO: 32 - protein, 19 aa, Homo sapiens
  misc_feature (1)..(19): Heavy chain secretion leader sequence (AA)
MDWTWRILFLVAAATGAHS

SEQ ID NO: 33 - DNA, 66 bp, Homo sapiens
  misc_feature (1)..(66): Light chain secretion leader sequence (DNA)
atggacatgagagtcctcgctcagctcctggggctcctgctgctctgtttcccaggtgccagatgt

SEQ ID NO: 34 - protein, 22 aa, Homo sapiens
  misc_feature (1)..(22): Light chain secretion leader sequence (AA)
MDMRVLAQLLGLLLLCFPGARC

(19) United States
(12) Patent Application Publication
(10) Pub. No.: US 2021/0379183 A1
(43) Pub. Date: Dec. 9, 2021

(54) METHODS OF TREATING IgA NEPHROPATHY WITH AN APRIL BINDING ANTIBODY

(71) Applicants: CHINOOK THERAPEUTICS, INC., Seattle, WA (US); ADURO BIOTECH HOLDINGS, EUROPE B.V., Oss (NL)

(72) Inventors: Teun VAN DE LAAR, Helvoirt (NL); Somayeh HONARMAND, Orinda, CA (US); John DULOS, Elst (NL); Eduard DE COCK, Beuningen (NL)

(73) Assignees: CHINOOK THERAPEUTICS, INC., Seattle, WA (US); ADURO BIOTECH HOLDINGS, EUROPE B.V., Oss (NL)

(21) Appl. No.: 17/334,590

(22) Filed: May 28, 2021

Related U.S. Application Data
(60) Provisional application No. 62/704,831, filed on May 29, 2020.

(51) Int. Cl. A61K 39/395; C07K 16/28; A61K 47/22; A61K 47/18; A61K 47/26

(52) U.S. Cl. CPC A61K 39/39591; C07K 16/2875; A61K 47/22; A61K 2039/54; A61K 47/26; C07K 2317/24; A61K 47/183

(57) ABSTRACT

Formulation and use of antibodies which bind human APRIL for the treatment of conditions related to IgA overproduction or deposition, including IgA Nephropathy.

### RELATED APPLICATION

This application claims the benefit of U.S. Provisional Application Ser. No. 62/704,831, filed May 29, 2020, from which priority is claimed and which is hereby incorporated by reference in its entirety including all tables, figures and claims.

### SEQUENCE LISTING

The instant application contains a Sequence Listing which has been submitted in ASCII format via EFS-Web and is hereby incorporated by reference in its entirety. Said ASCII copy, created on Aug. 13, 2021, is named ADR-1013-US_SeqListing.txt and is 30 kilobytes in size.

### FIELD OF THE INVENTION

The present invention relates to the use of isolated antibodies, including fragments thereof, which bind human APRIL, for the treatment of IgA Nephropathy.

### BACKGROUND OF THE INVENTION

APRIL is expressed as a type-II transmembrane protein, but unlike most other TNF family members it is mainly processed as a secreted protein and cleaved in the Golgi apparatus where it is cleaved by a furin convertase to release a soluble active form (Lopez-Fraga et al., 2001, EMBO Rep 2:945-51,). APRIL assembles as a non-covalently linked homo-trimer with similar structural homology in protein fold to a number of other TNF family ligands (Wallweber et al., 2004, Mol Biol 343, 283-90). APRIL binds two TNF receptors: B cell maturation antigen (BCMA) and transmembrane activator and calcium modulator and cyclophilin ligand interactor (TACI) (reviewed in Kimberley et al., 2009, J Cell Physiol. 218(1):1-8). In addition, APRIL has recently been shown to bind heparan sulphate proteoglycans (HSPGs) (Hendriks et al., 2005, Cell Death Differ 12, 637-48). APRIL has been shown to have a role in B cell signalling and drive both proliferation and survival of human and murine B cells in-vitro (reviewed in Kimberley et al., 2009, J Cell Physiol. 218(1):1-8).

APRIL is predominantly expressed by immune cell subsets such as monocytes, macrophages, dendritic cells, neutrophils, B-cells, and T-cells, many of which also express BAFF. In addition, APRIL can be expressed by non-immune cells such as osteoclasts, epithelial cells and a variety of tumour tissues (reviewed in Kimberley et al., 2009, J Cell Physiol. 218(1):1-8). In fact, APRIL was originally identified based on its expression in cancer cells (Hahne et al., 1998, J Exp Med 188, 1185-90). High expression levels of APRIL mRNA were found in a panel of tumour cell lines as well as human primary tumours such as colon, and a lymphoid carcinoma.

APRIL serum levels were found to be increased in patients suffering from IgA nephropathy (McCarthy et al., 2011, J. Clin. Invest. 121(10):3991-4002).

APRIL plays a crucial role in the survival and proliferative capacity of several B-cell malignancies, and potentially also some solid tumours. APRIL is also emerging as a key player in inflammatory diseases or autoimmunity. Thus, strategies to antagonize APRIL are a therapeutic goal for a number of these diseases. Indeed clinical studies targeting APRIL with TACI-Fc (Atacicept) are currently ongoing for treatment of several autoimmune diseases. However, TACI-Fc also targets BAFF, a factor involved in normal B-cell maintenance. Antibodies directed against APRIL have been described in WO9614328, WO2001/60397, WO2002/94192, WO9912965, WO2001/196528, WO9900518 and WO2010/100056. WO2010/100056 describes antibodies targeting APRIL specifically. The antibodies of WO2010/100056 fully block the binding of APRIL to TACI and at least partially to BCMA. Antibody hAPRIL.01A fully blocks the binding to both BCMA and TACI. The hAPRIL.01A antibody inhibited B-cell proliferation, survival and antigen-specific Immunoglobulin secretion in vitro and in vivo (Guadagnoli et al., 2011, Blood 117(25):6856-65). In addition, hAPRIL.01A inhibited proliferation and survival of malignant cells in in vitro and in vivo representative of human CLL and MM disease (Guadagnoli et al., 2011, Blood 117(25):6856-65; Lascano et al., 2013, Blood 122(24): 3960-3; Tai et al., 2014, ASH poster 2098). Finally, hAPRIL.01A inhibited the secretion of antigen-specific IgA (Guadagnoli et al., 2011, Blood 117(25):6856-65). Anti-APRIL antibodies, including those that fully block the binding to both BCMA and TACI, may be useful in treating IgA nephropathy, and there is a need to provide more efficacious formulations and dosing regimen to treat this disease.

### SUMMARY OF THE INVENTION

The present invention relates to anti-APRIL antibody formulations that are suitable for delivery by parenteral routes, and in particular intravenous and/or subcutaneous routes. The formulations described herein can provide high-concentration dosing solutions while maintaining acceptable viscosities, antibody solubility, levels of protein degradation and aggregation (particularly during long-term storage), and administration site pain resulting from certain inactive ingredients of the formulation.

In a first aspect, the present invention provides an antibody formulation, comprising:

an anti-APRIL antibody at a concentration of about 20 mg/mL to about 190 mg/mL;

about 10 mM L-histidine;

about 75 mM L-arginine;

about 3% wt % sorbitol;

about 0.01 wt % polysorbate 20; and

a pH of about 6.0 to about 6.6.

In various embodiments, the formulation exhibits one or more, and preferably 2, 3, or all 4, of the following characteristics:

has a viscosity of 16 cP or less,

does not comprise glutamic acid or its salt,

has an osmolality of between about 250 mOsm/kg to about 390 mOsm/kg, and

has an optical density at 330 nm (OD330) of about 1.0 or less.

In certain embodiments, the formulation maintains at least 96% purity of the anti-APRIL antibody following storage at 2-8° C. for 9 months following manufacture of the formulation; and preferably also maintains at least 95% purity of the anti-APRIL antibody following storage at 25° C. for 6 months following manufacture of the formulation.

Protein aggregation is generally believed to occur as the result of two instability factors: conformational and colloidal. Conformational stability is the difference in free energy between the folded and unfolded states of a protein. Although not directly measured as a value of energy, the Melting Temperature value of T_{M} or to another extent, the Aggregation Temperature, T_{Agg}, can qualitatively determine increased or decreased conformational stability between formulations. Colloidal stability is a result of balancing attractive and repulsive intermolecular interactions; that is, the less protein-protein interactions take place, the less likelihood there is for a sample to aggregate. The osmotic interaction second virial coefficient can provide a tool for predicting the aggregation propensity of proteins in a formulation state. In certain embodiments, the formulation of the invention has a second virial coefficient of about 2.5×10⁻⁵ mol·mL/g² or greater when measured at 25° C. The second virial coefficient may be measured as known in the art, for example, by static light scattering or membrane osmometry.

High concentration antibody formulations are often described as being “opalescent,” a property that results from turbidity in the sample and which may be a precursor to self-association and aggregation of the antibody. Measurement of optical density at 330 nm is reflective of this turbidity. In preferred embodiments, the formulation has an OD330 of about 0.8 or less.

In various embodiments, the anti-APRIL antibody of the formulation has a calculated isoelectric point (pI) of about 7.4 or greater. Protein pI is calculated using pK values of amino acids described in Bjellqvist et al., Electrophoresis 1993, 14, 1023-1031.

In preferred embodiments, the anti-APRIL antibody is at a concentration of about 150 mg/mL in the formulation. In particularly preferred embodiments, such a formulation has an osmolality of between about 290 mOsm/kg to about 390 mOsm/kg, and most preferably also an OD330 of about 0.8 or less.

In other preferred embodiments, the anti-APRIL antibody is at a concentration of about 20 mg/mL in the formulation. In particularly preferred embodiments, such a formulation has an osmolality of between about 293 mOsm/kg to about 333 mOsm/kg, and most preferably also an OD330 of about 0.8 or less.

In certain embodiments, the formulation is free of one or more of, and most preferably each of, glycine, carbonate, HEPES, phosphate, citrate, and acetate.

Preferably, the anti-APRIL antibody of the formulation is a humanized antibody comprising a heavy chain variable region.light chain variable region pair selected from the group consisting of VH11.VL15, VH12.VL15, VH13.VL15, VH14.VL15, VH14_1.VL15, VH14_1C.VL15, VH14_1D.VL15, VH14_1E.VL15, and VH14_1 G.VL15. These sequences are defined hereinafter. Most preferred is VH14_1 G.VL15.

The term “about” as used throughout this document refers to +/−10% of any given value, and preferably +/−5% of any given value.

In a related aspect, the present claims relate to a method of administering an anti-APRIL antibody to an individual in need thereof comprising administering the formulation described herein by subcutaneous injection into the individual.

In another related aspect, the present claims relate to a method of administering an anti-APRIL antibody to an individual in need thereof comprising administering the formulation described herein by intravenous infusion into the individual.

Various dosing schedules may be employed as described hereinafter. In certain embodiments, the method comprises repeating the infusion or subcutaneous administration on a weekly (“QW”) schedule for multiple cycles (e.g., 4 weeks, 6 weeks, 8 weeks, etc.). In other embodiments, the method comprises repeating the infusion or subcutaneous administration on a schedule of at least every two weeks (“biweekly as used herein” or “Q2W”) schedule for multiple cycles (e.g., 4 weeks, 6 weeks, 8 weeks, etc.). Alternatively, the method comprises repeating the infusion or subcutaneous administration on a schedule of at least every 4 weeks (“Q4W”) or once per month (“QMT”) schedule for multiple cycles (e.g., 8 weeks, 12 weeks, 16 weeks, etc.). In certain embodiments, a frontloading dosing schedule is used. In one example a loading dosing schedule comprising administration, either by intraveneous infusion or subcutaneous administration, repeating at least every two weeks for up to at least 4 weeks, is followed by a maintenance dosing schedule comprising administration, either by intraveneous infusion or subcutaneous administration, wherein the maintenance dosing schedule results in administration of less of the anti-APRIL antibody, either by each administration comprising less anti-APRIL antibody, or by administering at longer intervals than during the loading dosing schedule. In another example, a loading dosing schedule comprising administration, either by intraveneous infusion or subcutaneous administration, repeating at least daily and more preferably twice daily for up to at least 4 days, is followed by a maintenance dosing schedule comprising administration, either by intraveneous infusion or subcutaneous administration, such as on a QW, Q2W, Q4W, QM, etc. schedule. In one embodiment, the loading dosing schedule comprises administering the antibody by intraveneous infusion, and the maintenance dosing schedule comprises administering the antibody by subcutaneous injection. In another embodiment, both the loading dosing schedule and the maintenance dosing schedule comprises administering the antibody by subcutaneous injection. In another embodiment, both the loading dosing schedule and the maintenance dosing schedule comprises administering the antibody by intraveneous infusion. This is not meant to be an exhaustive list of dosing schedules.

By way of example only, the subcutaneous injection of the method comprises administering about 2 mL of the antibody formulation into the patient's preferred injection site (e.g. thigh, abdomen, upper arm, etc.). In preferred embodiments, the anti-APRIL antibody of the formulation is at a concentration of about 150 mg/mL, resulting in administration of about 300 mg of anti-APRIL antibody in a single injection. In certain embodiments, the subcutaneous injection of the method comprises administering about 4 mL (as a single injection or as 2×2 mL injections) of the antibody formulation of the anti-APRIL antibody at a concentration of about 150 mg/mL, resulting in administration of about 600 mg of anti-APRIL antibody. The volume of administration, and the number of injections required as part of a single administration, may be adjusted as necessary to achieve a total desired dose of between about 10 mg to about 1350 mg of the anti-APRIL antibody.

In certain other embodiments, the intravenous infusion of the method comprises: (a) diluting the formulation of the first aspect of the invention, and embodiments thereof, to a concentration of between about 0.1 mg/mL to about 10 mg/mL in 0.9% saline; and (b) administering a total dose of between about 10 mg to about 1350 mg of the anti-APRIL antibody to the individual in a single intravenous dose of the diluted formulation over a period of about 2 hours. Again, by way of example only, about 15 mL of a formulation at an anti-APRIL antibody concentration of about 20 mg/mL is added to about 235 mL of 0.9% saline to provide the intraveneous dose at a concentration of about 1.2 mg/mL.

In certain embodiments, the method of administering an anti-APRIL antibody to an individual in need thereof comprises administering the formulation described herein by a loading/maintenance administration protocol. Such a protocol may comprise a loading component of the protocol that comprises one or more administrations of the anti-APRIL antibody at a higher concentration than the anti-APRIL antibody concentration in the maintenance component of the loading/maintenance administration protocol; one or more administrations of the anti-APRIL antibody at a higher frequency than the frequency of administration of the anti-APRIL antibody in the maintenance component of the loading/maintenance administration protocol; and/or one or more administrations of the anti-APRIL antibody at a different route than the route of administration of the anti-APRIL antibody in the maintenance component of the loading/maintenance administration protocol.

By way of example only, the loading component of the loading/maintenance administration protocol may comprise one or more intravenous administrations of the anti-APRIL antibody and the maintenance component of the loading/maintenance administration protocol comprises one or more subcutaneous administrations of the anti-APRIL antibody. In such an example, the concentration of the loading administration(s) may be higher and/or the frequency of administration may be greater than is used in the maintenance administration(s).

In another example, the loading component of the loading/maintenance administration protocol may comprise one or more subcutaneous administrations of the anti-APRIL antibody and the maintenance component of the loading/maintenance administration protocol comprises one or more intravenous administrations of the anti-APRIL antibody. In such an example, the concentration of the loading administration(s) may be higher and/or the frequency of administration may be greater than is used in the maintenance administration(s).

In another example, the loading component of the loading/maintenance administration protocol may comprise one or more subcutaneous administrations of the anti-APRIL antibody and the maintenance component of the loading/maintenance administration protocol comprises one or more subcutaneous administrations of the anti-APRIL antibody. In such an example, the concentration of the loading administration(s) may be higher and/or the frequency of administration may be greater than is used in the maintenance administration(s).

In one embodiment, the loading dose comprises intraveneous infusion of 150 to 1350 mg of an anti-APRIL antibody, with at least one subsequent infusion of that amount at a first time interval, and the maintenance dose comprises administering either i) a lower amount of the anti-APRIL antibody administered at the first time interval after the last loading dose infusion, with at least one subsequent administration at the lower amount and the same time interval for at least 12 weeks, ii) the same amount of anti-APRIL antibody administered at a second time interval after the last loading dose infusion, with at least one subsequent administration at the same amount and the second time interval for at least 12 weeks, wherein the second time interval is longer than the first time interval, or iii) a lower amount of the anti-APRIL antibody administered at the second time interval after the last loading dose infusion, with at least one subsequent administration the same amount at the second time interval for a least 12 weeks, wherein the maintenance dosing may be by intraveneous infusion or by subcutaneous injection, preferably subcutaneous injection. In one embodiment, the loading dose comprises subcutaneous injection of 150 to 1350 mg of an anti-APRIL antibody, with at least one subsequent subcutaneous injection of that amount at a first time interval, and the maintenance dose comprises administering either i) a lower amount of the anti-APRIL antibody administered at the first time interval after the last loading dose infusion, with at least one subsequent administration at the lower amount and the same time interval for at least 12 weeks, ii) the same amount of anti-APRIL antibody administered at a second time interval after the last loading dose infusion, with at least one subsequent administration at the same amount and the second time interval for at least 12 weeks, wherein the second time interval is longer than the first time interval, or iii) a lower amount of the anti-APRIL antibody administered at the second time interval after the last loading dose infusion, with at least one subsequent administration the same amount at the second time interval for a least 12 weeks, wherein the maintenance dosing may be by intraveneous infusion or by subcutaneous injection.

In another embodiment of the invention, an article of manufacture containing materials useful for the treatment of the disorders described above is provided. The article of manufacture comprises a container, a label and a package insert. Suitable containers include, for example, bottles, vials, syringes (pre-filled or filled from containers at the time of administration), autoinjectors, injector pens, etc. The containers may be formed from a variety of materials such as glass or plastic. The container holds a composition which is effective for treating the condition and may have a sterile access port (for example, the container may be an intravenous solution bag or a vial having a stopper pierceable by a hypodermic injection needle). At least one active agent in the article of manufacture is a container comprising an anti-APRIL antibody composition according to the present invention. In certain embodiments, the label on, or associated with, the container indicates that the composition is used for treating the condition of choice, e.g. IgA nephropathy for example. The article of manufacture may further comprise a second container comprising a pharmaceutically-acceptable buffer, such as phosphate-buffered saline, Ringer's solution and dextrose solution. It may further include other materials desirable from a commercial and user standpoint, including other buffers, diluents, filters, needles, and syringes. In addition, the article of manufacture may comprise a package inserts with instructions for use. The formulation of the present invention may be provided in a variety of forms, such as a single-use or multi-use vial comprising the antibody formulation or a pre-filled syringe, autoinjector, or injector pen comprising the antibody formulation. The concentration of the anti-APRIL antibody in such a container may be between about 20 mg/mL to about 190 mg/mL, and most preferably about 150 mg/mL. The volume of the formulation in such a container may be between 0.5 mL and 50 mL; preferably between 1 mL and 10 mL, and most preferably 1 mL, 2 mL, 3 mL, 4 mL, or 5 mL.

### BRIEF DESCRIPTION OF THE SEQUENCES

The sequences presented in the sequence listing relate to the amino acid sequences and encoding DNA sequences of V_{H} and V_{L} domains and of heavy and light chains of preferred antibodies for the formulations and methods described herein, including the amino acid sequences and encoding DNA sequences of the preferred heavy and light chains of the preferred antibody described herein. In addition, the amino acid sequences of the CDRs of both the heavy and light chains of the antibodies described herein are presented. Table 1 below correlates the sequence IDs to their respective sequence.

TABLE 1
Sequence Listing

| SEQ ID NO: | Description |
|---|---|
| 1 | anti-hAPRIL heavy chain CDR1 (AA) |
| 2 | anti-hAPRIL heavy chain CDR2 (AA) |
| 3 | anti-hAPRIL heavy chain CDR3 (AA) |
| 4 | anti-hAPRIL light chain CDR1 (AA) |
| 5 | anti-hAPRIL light chain CDR2 (AA) |
| 6 | anti-hAPRIL light chain CDR3 (AA) |
| 7 | VH11 heavy chain variable region (DNA) |
| 8 | VH11 heavy chain variable region (AA) |
| 9 | VH12 heavy chain variable region (DNA) |
| 10 | VH12 heavy chain variable region (AA) |
| 11 | VH13 heavy chain variable region (DNA) |
| 12 | VH13 heavy chain variable region (AA) |
| 13 | VH14 heavy chain variable region (DNA) |
| 14 | VH14 heavy chain variable region (AA) |
| 15 | VH14_1 heavy chain variable region (DNA) |
| 16 | VH14_1 heavy chain variable region (AA) |
| 17 | VH14_1C heavy chain variable region (DNA) |
| 18 | VH14_1C heavy chain variable region (AA) |
| 19 | VH14_1D heavy chain variable region (DNA) |
| 20 | VH14_1D heavy chain variable region (AA) |
| 21 | VH14_1E heavy chain variable region (DNA) |
| 22 | VH14_1E heavy chain variable region (AA) |
| 23 | VH14_1G heavy chain variable region (DNA) |
| 24 | VH14_1G heavy chain variable region (AA) |
| 25 | VL15 light chain variable region (DNA) |
| 26 | VL15 light chain variable region (AA) |
| 27 | VH14_1G heavy chain (DNA) |
| 28 | VH14_1G heavy chain (AA) |
| 29 | VL15 light chain (DNA) |
| 30 | VL15 light chain (AA) |
| 31 | Heavy chain secretion leader sequence (DNA) |
| 32 | Heavy chain secretion leader sequence (AA) |
| 33 | Light chain secretion leader sequence (DNA) |
| 34 | Light chain secretion leader sequence (AA) |

### BRIEF DESCRIPTION OF THE FIGURES

FIG. 1 depicts tabular results of turbidity, visual appearance and pH for various VH14_1 G.VL15 antibody formulations following temperature stress, freeze/thaw and shaking stress.

FIG. 2 depicts tabular results of percent purity for various VH14_1 G.VL15 antibody formulations following temperature stress, freeze/thaw and shaking stress as measured by SE-UPLC.

FIG. 3 depicts a graphical representation of SE-UPLC percent purity measured by peak areas of various VH14_1 G.VL15 antibody formulations following 12 weeks storage at −70° C.

FIG. 4 depicts a graphical representation of SE-UPLC percent purity measured by peak areas of various VH14_1 G.VL15 antibody formulations following 12 weeks storage at 2 to 8° C.

FIG. 5 depicts a graphical representation of SE-UPLC percent purity measured by peak areas of various VH14_1 G.VL15 antibody formulations following 12 weeks storage at 25° C.

FIG. 6 depicts a graphical representation of SE-UPLC percent purity measured by peak areas of various VH14_1 G.VL15 antibody formulations following 12 weeks storage at 45° C.

FIG. 7 depicts tabular results of percent purity for various VH14_1 G.VL15 antibody formulations following temperature stress, freeze/thaw and shaking stress as measured by CEX-UPLC.

FIG. 8 depicts ln purity (%) vs. time (days) for various VH14_1 G.VL15 antibody formulations at 25° C.

FIG. 9 depicts Arrhenius relationship plots (ln k_{obs} vs 1/T (Kelvin)) at 2-8° C., 25° C., and 45° C. for four VH14_1 G.VL15 antibody formulations.

FIG. 10 depicts ln purity (%) vs. time (days) for four VH14_1 G.VL15 antibody formulations at 25° C.

FIG. 11 depicts Arrhenius relationship plots (ln k_{obs} vs 1/T (Kelvin)) at 25° C., and 45° C. for various VH14_1 G.VL15 antibody formulations.

FIG. 12 depicts hydrodynamic radii (in nm) and % Mass of the population of species in various VH14_1 G.VL15 antibody testing samples.

FIG. 13 depicts tabular results of turbidity, visual appearance and pH for various VH14_1 G.VL15 antibody formulations following temperature stress.

FIG. 14 depicts tabular HIAC particle counting results for various VH14_1 G.VL15 antibody formulations following temperature stress.

FIG. 15 depicts tabular results of percent purity for various VH14_1 G.VL15 antibody formulations following temperature stress as measured by SE-HPLC.

FIG. 16 depicts tabular results of percent purity for various VH14_1 G.VL15 antibody formulations following temperature stress as measured by CE-HPLC.

FIG. 17 depicts ln purity (%) vs. time (days) for various VH14_1 G.VL15 antibody formulations at 25° C.

FIG. 18 depicts ln purity (%) vs. time (days) for various VH14_1 G.VL15 antibody formulations at 45° C.

FIG. 19 depicts Arrhenius relationship plots (ln k_{obs} vs 1/T (Kelvin)) at 25° C., and 45° C. for various VH14_1 G.VL15 antibody formulations.

FIG. 20 depicts ln purity (%) vs. time (days) for various VH14_1 G.VL15 antibody formulations at 5° C.

FIG. 21 depicts ln purity (%) vs. time (days) for various VH14_1 G.VL15 antibody formulations at 25° C.

FIG. 22 depicts ln purity (%) vs. time (days) for various VH14_1 G.VL15 antibody formulations at 45° C.

FIG. 23 depicts Arrhenius relationship plots (ln k_{obs} vs 1/T (Kelvin)) at 5° C., 25° C., and 45° C. for various VH14_1 G.VL15 antibody formulations.

FIG. 24 depicts a clinical trial protocol to evaluate the safety, tolerability, PK, and PD of IV administered VH14_1G.VL15.

FIG. 25 depicts mean serum BION-1301 concentrations+/−SD vs nominal time following IV administration of various doses of BION-1301.

FIG. 26 depicts mean free APRIL concentrations in serum as a percent of initial baseline concentration following IV administration of various doses of BION-1301.

FIGS. 27A-F depict mean change in serum immunoglobulin IgA, IgG, and IgM concentrations in serum as a percent of initial baseline concentration following IV administration of various doses of BION-1301.

FIG. 28A depicts percent change in serum immunoglobulin IgA, IgG, and IgM concentrations in serum as a percent of initial baseline concentration at day 29 following IV administration of various doses of BION-1301.

FIG. 28B depicts percent change in serum immunoglobulin IgA, IgG, and IgM concentrations in serum as a percent of initial baseline concentration at day 85 following IV administration of various doses of BION-1301.

FIG. 29 depicts a clinical trial protocol to evaluate the safety, tolerability, PK, and PD of IV administered vs SC administered BION-1301.

FIG. 30 depicts the mean (±SD) serum concentration of BION-1301 vs. time following a single IV or SC administration of 300 mg BION-1301 (semi-log scale).

FIG. 31A depicts the mean (±SD) fAPRIL concentrations after single-dose IV or SC administration of 300 mg BION-1301.

FIG. 31B depicts the mean (±SD) percent change relative to the baseline of fAPRIL after single-dose IV or SC administration of 300 mg BION-1301.

FIG. 32A-C depicts the mean % change relative to baseline in serum immunoglobulin levels over time (32A=IgA, 32B=IgG, 32C=IgM) after single-dose IV or SC administration of 300 mg BION-1301.

FIG. 33 shows reductions in serum IgA and Gd-IgA1 in a single ascending dose (SAD) and multiple ascending dose (MAD) study of BION-1301 administered by intravenous (IV) infusion in healthy human volunteers (ADU-CL-19; ClinicalTrials.gov Identifier: NCT03945318).

FIG. 34 shows changes in free APRIL levels, Gd-IgA1 levels, mesangial cell proliferation and proteinuria in IgAN patients following treatment with BION-1301.

### DETAILED DESCRIPTION

The invention thus relates to antibodies as described herein, uses and formulations thereof, that are efficious in treating IgA Nephropathy. The antibodies described herein are exemplified using the anti-hAPRIL antibody having amino acid sequence of SEQ ID NO: 28 for the heavy chain and SEQ ID NO: 30 for the light chain (also referred to as VH14_1G.VL15, or as used in clinical trials is also referred to as BION-1301). This antibody blocks the binding of human APRIL to human B cell maturation antigen (BCMA) and transmembrane activator and calcium modulator and cyclophilin ligand interactor (TACI), and has been shown to significantly reduce the levels of IgA in healthy volunteers. This reduction in IgA levels is expected to be similar in subjects having IgA nephropathy, and is therefore expected to have a significant therapeutic benefit. Additional features and discussion of antibodies useful in the formulation and methods described herein can be found in International Patent Application Number WO2016110587, the disclosure of which is hereby incorporated by reference as it relates to anti-APRIL antibodies useful in the treatment of IgA nephropathy. The formulations and methods as provided herein are expected to provide a stable formulation of the anti-hAPRIL antibody for treating IgA nephropathy.

Within the description of the present invention at least 90% sequence similarity should be understood as meaning more preferably at least 95%, such as at least 99% sequence similarity.

As the skilled person will understand, “sequence similarity” refers to the extent to which individual nucleotide or peptide sequences are alike. The extent of similarity between two sequences is based on the extent of identity combined with the extent of conservative changes. The percentage of “sequence similarity” is the percentage of amino acids or nucleotides which is either identical or conservatively changed viz. “sequence similarity”=(% sequence identity)+(% conservative changes).

For the purpose of this invention “conservative changes” and “identity” are considered to be species of the broader term “similarity”. Thus, whenever the term sequence “similarity” is used it embraces sequence “identity” and “conservative changes”. According to certain embodiments the conservative changes are disregarded and the % sequence similarity refers to % sequence identity.

The term “sequence identity” is known to the skilled person. In order to determine the degree of sequence identity shared by two amino acid sequences or by two nucleic acid sequences, the sequences are aligned for optimal comparison purposes (e.g., gaps can be introduced in the sequence of a first amino acid or nucleic acid sequence for optimal alignment with a second amino or nucleic acid sequence). Such alignment may be carried out over the full lengths of the sequences being compared. Alternatively, the alignment may be carried out over a shorter comparison length, for example over about 20, about 50, about 100 or more nucleic acids/bases or amino acids.

The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The degree of identity shared between sequences is typically expressed in terms of percentage identity between the two sequences and is a function of the number of identical positions shared by identical residues in the sequences (i.e., % identity=number of identical residues at corresponding positions/total number of positions×100). Preferably, the two sequences being compared are of the same or substantially the same length.

The percentage of “conservative changes” may be determined similar to the percentage of sequence identity. However, in this case changes at a specific location of an amino acid or nucleotide sequence that are likely to preserve the functional properties of the original residue are scored as if no change occurred.

For amino acid sequences the relevant functional properties are the physico-chemical properties of the amino acids. A conservative substitution for an amino acid in a polypeptide of the invention may be selected from other members of the class to which the amino acid belongs. For example, it is well-known in the art of protein biochemistry that an amino acid belonging to a grouping of amino acids having a particular size or characteristic (such as charge, hydrophobicity and hydrophilicity) can be substituted for another amino acid without substantially altering the activity of a protein, particularly in regions of the protein that are not directly associated with biological activity (see, e.g., Watson, et al., Molecular Biology of the Gene, The Benjamin/Cummings Pub. Co., p. 224 (4th Edition 1987)). For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Conservative substitutions include, for example, Lys for Arg and vice versa to maintain a positive charge; Glu for Asp and vice versa to maintain a negative charge; Ser for Thr and vice versa so that a free —OH is maintained; and Gln for Asn and vice versa to maintain a free —NH₂.

For nucleotide sequences the relevant functional properties is mainly the biological information that a certain nucleotide carries within the open reading frame of the sequence in relation to the transcription and/or translation machinery. It is common knowledge that the genetic code has degeneracy (or redundancy) and that multiple codons may carry the same information in respect of the amino acid for which they code. For example in certain species the amino acid leucine is coded by UUA, UUG, CUU, CUC, CUA, CUG codons (or TTA, TTG, CTT, CTC, CTA, CTG for DNA), and the amino acid serine is specified by UCA, UCG, UCC, UCU, AGU, AGC (or TCA, TCG, TCC, TCT, AGT, AGC for DNA). Nucleotide changes that do not alter the translated information are considered conservative changes.

For the present invention it is most preferred to use BLAST (Basic Local Alignment Tool) to determine the percentage identity and/or similarity between nucleotide or amino acid sequences.

Queries using the BLASTn, BLASTp, BLASTx, tBLASTn and tBLASTx programs of Altschul et al. (1990) may be posted via the online versions of BLAST accessible via http://www.ncbi.nlm.nih.gov. Alternatively a standalone version of BLAST {e.g., version 2.2.29 (released 3 Jan. 2014)) downloadable also via the NCBI internet site may be used. Preferably BLAST queries are performed with the following parameters. To determine the percentage identity and/or similarity between amino acid sequences: algorithm: blastp; word size: 3; scoring matrix: BLOSUM62; gap costs: Existence: 11, Extension: 1; compositional adjustments: conditional compositional score matrix adjustment; filter: off; mask: off. To determine the percentage identity and/or similarity between nucleotide sequences: algorithm: blastn; word size: 11; max matches in query range: 0; match/mismatch scores: 2, −3; gap costs: Existence: 5, Extension: 2; filter: low complexity regions; mask: mask for lookup table only.

The percentage of “conservative changes” may be determined similar to the percentage of sequence identity with the aid of the indicated algorithms and computer programs. Some computer programs, e.g., BLASTp, present the number/percentage of positives (=similarity) and the number/percentage of identity. The percentage of conservative changes may be derived therefrom by subtracting the percentage of identity from the percentage of positives/similarity (percentage conservative changes=percentage similarity—percentage identity).

According to a further aspect, the invention relates to an isolated polynucleotide encoding a V_{H} domain and/or a V_{L} domain of an antibody, or a heavy chain and/or light chain of the antibody, according to the invention. A polynucleotide sequence encoding the V_{H} domain preferably is a polynucleotide sequence having at least 90% sequence similarity with a polynucleotide sequence selected from the group consisting of SEQ ID NO: 7, 9, 11, 13, 15, 17, 19, 21 and 23, preferably SEQ ID NO: 13, 15 or 23, more preferably SEQ ID NO: 23. A polynucleotide sequence encoding the V_{L} domain preferably is a polynucleotide sequence having at least 90% sequence similarity with a polynucleotide sequence of SEQ ID NO: 25. A polynucleotide sequence encoding the heavy chain preferably is a polynucleotide sequence having at least 90% sequence similarity with a polynucleotide sequence of SEQ ID NO: 27. A polynucleotide sequence encoding the light chain preferably is a polynucleotide sequence having at least 90% sequence similarity with a polynucleotide sequence of SEQ ID NO: 29.

The invention further relates to an expression unit comprising a number of expression vectors, comprising a number of polynucleotides according to the invention under the control of suitable regulatory sequences, wherein the number of polynucleotides encode the V_{H} domain or heavy chain and the V_{L} domain or light chain of an antibody according to the invention. The expression unit may be designed such that the polynucleotide sequence coding for the V_{H} domain or heavy chaining and the polynucleotide sequence coding for V_{L} domain or light chain may be on the same expression vector. Thus the expression unit may comprise a single vector. Alternatively the polynucleotide sequence coding for the V_{H} domain or heavy chain and the polynucleotide sequence coding for the V_{L} domain or light chain may be on different expression vectors. In such embodiments the expression unit will comprise a plurality, such as for example 2, expression vectors.

A further aspect of the invention relates to a host cell comprising a number of polynucleotides of the invention and/or an expression unit of the invention. The expression unit preferably is an expression unit comprising an expression vector comprising both a polynucleotide sequence coding for the V_{H} domain or heavy chain and a polynucleotide sequence coding for the V_{L} domain or light chain.

Therapy

The formulations and methods of use of the antibodies of the present invention are suitable for treatment of a condition known or expected to be ameliorated by blocking the interaction of human APRIL with BCMA and/or TACI. As is already known in the art, blocking the interaction of human APRIL with BCMA and/or TACI inhibits immune cell proliferation and/or survival and thus may be of value for the treatment of conditions where such blocking of immune cell proliferation and/or survival is beneficial, such as inflammatory diseases, diseases mediated by Ig secretion and/or autoimmune diseases. Blocking of the interaction of human APRIL with BCMA and/or TACI may also be beneficial in the treatment of cancer.

Also, the antibodies of the invention may be beneficial in the treatment of other conditions wherein lowering of Immunoglobulin levels, such as IgA, including IgA1 or IgA2, IgG, IgM, Gd-IgA, levels, is beneficial, such as conditions associated with Ig secretion, in particular IgA secretion, Ig overproduction, such as IgA, including IgA1 or IgA2, IgG, IgM, Gd-IgA over production, in particular IgA overproduction, or Ig deposition, in particular IgA deposition. Examples of such conditions include, but are not limited to IgA nephropathy and other forms of glomerulonephritis, celiac disease, pemphigoid diseases, Henloch-Schönlein purpura, and other autoimmune diseases that are associated with Ig deposition. The formulations and methods of use of the anti-hAPRIL antibodies as described herein are particularly suited to the treatment of IgA nephropathy.

IgA nephropathy (IgAN) is the leading cause of primary glomerulonephritis (Berthelot L, et al., 2015, Kidney Int, 88:815-22). Prognosis for patients with IgAN is variable and depends on several factors. For patients with a mild or moderate proteinuria level and normal renal function at biopsy, 2.8% developed end-stage renal disease (ESRD) at 25 years of follow-up (Knoop T, et al., 2017, Nephrol Dial Transplant 32:1841-50), resulting in dialysis or kidney transplant. For the general IgAN population, it is reported that between 14% and 39% develop ESRD within 20 years from diagnosis (Berthoux F C, et al., 2008, Semin Nephrol 28:4-9; Manno C, et al., 2007, Am J Kidney Dis, 49:763-75). A critical early step in the pathology of IgAN is the generation of autoantibodies to galactose-deficient IgA1 (gd-IgA1), leading to the formation of immune-complexes which cause inflammation, mesangial cell proliferation, and complement activation that results in kidney damage. APRIL binds to BCMA and TACI to drive proliferation and survival of human plasmablasts/plasma cells (O'Connor B P, et al., 2004, J Exp Med, 199:91-8; Moreaux J, et al., 2007, Haematologica, 92:803-11). APRIL contributes to IgAN by promoting B-cell class switch to IgA-producing plasma cells (He B, et al., 2010, Nat Immunol 11:836-45) Importantly, anti-APRIL antibody decreases kidney damage, serum IgA, IgA deposits and proteinuria in an IgAN murine model.

Serum Gd-IgA1 levels are reportedly significantly higher in IgAN patients than disease controls and healthy controls. In patients with IgAN, serum Gd-IA1 levels were significantly correlated with estimated glomerular filtration rate, serum IgA level, and tubular atrophy/interstitial fibrosis. CKD progression was more frequent in IgAN patients with higher serum Gd-IgA1 levels than in those with lower serum Gd-IgA1 levels. Cox proportional hazard models showed that high GdIgA1 level was an independent risk factor for CKD progression after adjusting for several confounders. Kim et al., J. Clin. Med. 2020 Nov. 4; 9(11):3549. doi: 10.3390/jcm9113549.

A humanized APRIL antagonistic monoclonal antibody (as described herein) is in development for the treatment of IgAN, having undergone clinical trials in healthy volunteers (see clinicaltrials.gov NCT03945318). Blockade of APRIL by anti-hAPRIL antibody has been shown to significantly lower IgA and IgM and to a lesser extent IgG in healthy cynomolgus monkeys, and has shown similar results in the healthy human volunteers. In addition, this blockade reduced Gd-IgA1 in healthy human volunteers. Consequently, it is expected that blockade of APRIL in patients with IgAN will lead to a reduction in levels of IgA, IgG, and IgM and corresponding reductions in gd-IgA1, auto-antibodies to gd-IgA1, immune complex deposition and renal damage.

Myette et al. (2019, Kidney International 96(1):104-116) demonstrates the efficacy of a mouse anti-APRIL antibody in a mouse model for IgA nephropathy, and the human antibody VIS649 is part of a Phase 2 clinical trial (clinicaltrials.gov NCT04287985).

### General Definitions

The term “antibody” refers to any form of antibody that exhibits the desired biological activity, such as inhibiting binding of a ligand to its receptor, or by inhibiting ligand-induced signaling of a receptor. In the present case the biological activity comprises blocking of the binding of APRIL to its receptors BCMA and/or TACI. Thus, “antibody” is used in the broadest sense and specifically covers, but is not limited to, monoclonal antibodies (including full length monoclonal antibodies) and multispecific antibodies (e.g., bispecific antibodies) such as based on the Duobody® technology (Genmab) or Hexabody® technology (Genmab) or antibody fragment.

“Antibody fragment” and “antibody binding fragment” mean antigen-binding fragments and analogues of an antibody, typically including at least a portion of the antigen binding or variable regions (e.g. one or more CDRs) of the parental antibody. An antibody fragment retains at least some of the binding specificity of the parental antibody. Typically, an antibody fragment retains at least 10% of the parental binding activity when that activity is expressed on a molar basis. Preferably, an antibody fragment retains at least 20%, 50%, 70%, 80%, 90%, 95% or 100% or more of the parental antibody's binding affinity for the target. Examples of antibody fragments include, but are not limited to, Fab, Fab′, F(ab′)2, and Fv fragments; diabodies; linear antibodies; single-chain antibody molecules, e.g., sc-Fv, unibodies (technology from Genmab); nanobodies (technology from Ablynx); domain antibodies (technology from Domantis); and multispecific antibodies formed from antibody fragments. Engineered antibody variants are reviewed in Holliger and Hudson, 2005, Nat. Biotechnol. 23:1126-1136.

An “Fab fragment” is comprised of one light chain and the C_{H}1 and variable regions of one heavy chain. The heavy chain of a Fab molecule cannot form a disulfide bond with another heavy chain molecule.

An “Fc” region contains two heavy chain fragments comprising the C_{H}1 and C_{H}2 domains of an antibody. The two heavy chain fragments are held together by two or more disulfide bonds and by hydrophobic interactions of the C_{H}3 domains.

An “Fab′ fragment” contains one light chain and a portion of one heavy chain that contains the V_{H} domain and the C_{H}1 domain and also the region between the C_{H}1 and C_{H}2 domains, such that an interchain disulfide bond can be formed between the two heavy chains of two Fab′ fragments to form a F(ab′)₂ molecule.

An “F(ab′)₂ fragment” contains two light chains and two heavy chains containing a portion of the constant region between the C_{H}1 and C_{H}2 domains, such that an interchain disulfide bond is formed between the two heavy chains. A F(ab′)₂ fragment thus is composed of two Fab′ fragments that are held together by a disulfide bond between the two heavy chains.

The “Fv region” comprises the variable regions from both the heavy and light chains, but lacks the constant regions.

A “single-chain Fv antibody” (or “scFv antibody”) refers to antibody fragments comprising the V_{H} and V_{L} domains of an antibody, wherein these domains are present in a single polypeptide chain. Generally, the Fv polypeptide further comprises a polypeptide linker between the V_{H} and V_{L} domains which enables the scFv to form the desired structure for antigen binding. For a review of scFv, see Pluckthun, 1994, The Pharmacology of Monoclonal Antibodies, vol. 113, Rosenburg and Moore eds. Springer-Verlag, New York, pp. 269-315. See also, International Patent Application Publication No. WO 88/01649 and U.S. Pat. Nos. 4,946,778 and 5,260,203.

A “diabody” is a small antibody fragment with two antigen-binding sites. The fragments comprises a heavy chain variable domain (V_{H}) connected to a light chain variable domain (V_{L}) in the same polypeptide chain (V_{H}-V_{L} or V_{L}-V_{H}). By using a linker that is too short to allow pairing between the two domains on the same chain, the domains are forced to pair with the complementary domains of another chain and create two antigen-binding sites. Diabodies are described more fully in, e.g., EP 404,097; WO 93/11161; and Holliger et al., 1993, Proc. Natl. Acad. Sci. USA 90: 6444-6448.

“Duobodies” are bispecific antibodies with normal IgG structures (Labrijn et al., 2013, Proc. Natl. Acad. Sci. USA 110 (13): 5145-5150).

“Hexabodies” are antibodies that while retaining regular structure and specificity have an increased killing ability (Diebolder et al., 2014, Science 343(6176):1260-3).

A “domain antibody fragment” is an immunologically functional immunoglobulin fragment containing only the variable region of a heavy chain or the variable region of a light chain. In some instances, two or more V_{H} regions are covalently joined with a peptide linker to create a bivalent domain antibody fragment. The two V_{H} regions of a bivalent domain antibody fragment may target the same or different antigens.

An antibody fragment of the invention may comprise a sufficient portion of the constant region to permit dimerization (or multimerization) of heavy chains that have reduced disulfide linkage capability, for example where at least one of the hinge cysteines normally involved in inter-heavy chain disulfide linkage is altered as described herein. In another embodiment, an antibody fragment, for example one that comprises the Fc region, retains at least one of the biological functions normally associated with the Fc region when present in an intact antibody, such as FcRn binding, antibody half life modulation, ADCC (antibody dependent cellular cytotoxicity) function, and/or complement binding (for example, where the antibody has a glycosylation profile necessary for ADCC function or complement binding).

The term “chimeric” antibody refers to antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (See, for example, U.S. Pat. No. 4,816,567 and Morrison et al., 1984, Proc. Natl. Acad. Sci. USA 81:6851-6855).

As used herein, the term “humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The humanized forms of rodent antibodies will essentially comprise the same CDR sequences of the parental rodent antibodies, although certain amino acid substitutions may be included to increase affinity, increase stability of the humanized antibody, or for other reasons.

The antibodies of the present invention also include antibodies with modified (or blocked) Fc regions to provide altered effector functions. See, e.g. U.S. Pat. No. 5,624,821; WO2003/086310; WO2005/120571; WO2006/0057702; Presta, 2006, Adv. Drug Delivery Rev. 58:640-656. Such modification can be used to enhance or suppress various reactions of the immune system, with possible beneficial effects in diagnosis and therapy. Alterations of the Fc region include amino acid changes (substitutions, deletions and insertions), glycosylation or deglycosylation, and adding multiple Fc. Changes to the Fc can also alter the half-life of antibodies in therapeutic antibodies, and a longer half-life would result in less frequent dosing, with the concomitant increased convenience and decreased use of material. See Presta, 2005, J. Allergy Clin. Immunol.116:731 at 734-35.

The antibodies of the present invention also include antibodies with intact Fc regions that provide full effector functions, e.g. antibodies of isotype IgG1, which induce complement-dependent cytotoxicity (CDC) or antibody dependent cellular cytotoxicity (ADCC) in the a targeted cell.

The antibodies may also be conjugated (e.g., covalently linked) to molecules that improve stability of the antibody during storage or increase the half-life of the antibody in vivo. Examples of molecules that increase the half-life are albumin (e.g., human serum albumin) and polyethylene glycol (PEG). Albumin-linked and PEGylated derivatives of antibodies can be prepared using techniques well known in the art. See, e.g. Chapman, 2002, Adv. Drug Deliv. Rev. 54:531-545; Anderson and Tomasi, 1988, J. Immunol. Methods 109:37-42; Suzuki et al., 1984, Biochim. Biophys. Acta 788:248-255; and Brekke and Sandlie, 2003, Nature Rev. 2:52-62.

The term “hypervariable region,” as used herein, refers to the amino acid residues of an antibody which are responsible for antigen-binding. The hypervariable region comprises amino acid residues from a “complementarity determining region” or “CDR,” defined by sequence alignment, for example residues 24-34 (L1), 50-56 (L2) and 89-97 (L3) in the light chain variable domain and 31-35 (H1), 50-65 (H2) and 95-102 (H3) in the heavy chain variable domain (see Kabat et al., 1991, Sequences of proteins of Immunological Interest, 5th Ed. Public Health Service, National Institutes of Health, Bethesda, Md.) and/or those residues from a “hypervariable loop” (HVL), as defined structurally, for example, residues 26-32 (L1), 50-52 (L2) and 91-96 (L3) in the light chain variable domain and 26-32 (H1), 53-55 (H2) and 96-101 (H3) in the heavy chain variable domain (see Chothia and Leskl, 1987, J. Mol. Biol. 196:901-917).

“Framework” or “FR” residues or sequences are those variable domain residues or sequences other than the CDR residues as herein defined.

The antibody of the invention according to certain embodiments may be an isolated antibody. An “isolated” antibody is one that has been identified and separated and/or recovered from a component of its natural environment. Contaminant components of its natural environment are materials that would interfere with diagnostic or therapeutic uses for the antibody, and may include enzymes, hormones, and other proteinaceous or non-proteinaceous solutes. In some embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

An “isolated” nucleic acid molecule is a nucleic acid molecule that is identified and separated from at least one contaminant nucleic acid molecule with which it is ordinarily associated in the natural source of the antibody nucleic acid. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes a nucleic acid molecule contained in cells that ordinarily express the antibody where, for example, the nucleic acid molecule is in a chromosomal location different from that of natural cells.

The term “monoclonal antibody” when used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., 1975, Nature 256:495, or may be made by recombinant DNA methods (see, for example, U.S. Pat. No. 4,816,567). The “monoclonal antibodies” may also be isolated from phage antibody libraries using the techniques described in Clackson et al., 1991, Nature 352:624-628 and Marks et al., 1991, J. Mol. Biol. 222:581-597, for example. The monoclonal antibodies herein specifically include “chimeric” antibodies.

As used herein, the term “immune cell” includes cells that are of hematopoietic origin and that play a role in the immune response. Immune cells include lymphocytes, such as B cells and T cells, natural killer cells, myeloid cells, such as monocytes, macrophages, eosinophils, mast cells, basophils, and granulocytes.

As used herein, an “immunoconjugate” refers to an anti-human APRIL antibody, or a fragment thereof, conjugated to a therapeutic moiety, such as a bacterial toxin, a cytotoxic drug or a radiotoxin. Toxic moieties can be conjugated to antibodies of the invention using methods available in the art.

As used herein, a sequence “variant” or “variant sequence” refers to a sequence that differs from the disclosed sequence at one or more amino acid residues but which retains the biological activity of the parent molecule. The invention includes the variants of antibodies explicitly disclosed by the various sequences. For the VH domain CDR1, CDR2 and CDR3 sequences, according to some embodiments, variant sequences may comprise up to 6 amino acid substitutions, such as 1, 2, 3, 4, 5 or 6 amino acid substitutions, for the CDR1, CDR2 and CDR3 sequences taken together. Similarly for the VL domain CDR1, CDR2 and CDR3 sequences, according to some embodiments, variant sequences may comprise up to 6 amino acid substitutions, such as 1, 2, 3, 4, 5 or 6 amino acid substitutions, for the CDR1, CDR2 and CDR3 sequences taken together.

“Conservatively modified variants” or “conservative amino acid substitution” refers to substitutions of amino acids are known to those of skill in this art and may be made generally without altering the biological activity of the resulting molecule. Those of skill in this art recognize that, in general, single amino acid substitutions in non-essential regions of a polypeptide do not substantially alter biological activity (see, e.g., Watson, et al., Molecular Biology of the Gene, The Benjamin/Cummings Pub. Co., p. 224 (4th Edition 1987)).

As used herein, the term “about” refers to a value that is within an acceptable error range for the particular value as determined by one of ordinary skill in the art, which will depend in part on how the value is measured or determined, i.e. the limitations of the measurement system. For example, “about” can mean within 1 or more than 1 standard deviation per the practice in the art. Alternatively, “about” or “comprising essentially of” can mean a range of up to 20%. Furthermore, particularly with respect to biological systems or processes, the terms can mean up to an order of magnitude or up to 5-fold of a value. When particular values are provided in the application and claims, unless otherwise stated, the meaning of “about” or “comprising essentially of” should be assumed to be within an acceptable error range for that particular value.

The term “a number of” should be understood as meaning one or more. Depending on the context of its use “a number of” may refer to any suitable number selected from 1, 2, 3, 4, 5, 6, 7, 8, 9, 10. According to certain embodiments “a number of” may have the meaning of “a plurality”. Depending on the context of its use “a plurality” may refer to any suitable number selected from 2, 3, 4, 5, 6, 7 8, 9, 10.

“Specifically” binds, when referring to a ligand/receptor, antibody/antigen, or other binding pair, indicates a binding reaction which is determinative of the presence of the protein, e.g., APRIL, in a heterogeneous population of proteins and/or other biologics. Thus, under designated conditions, a specified ligand/antigen binds to a particular receptor/antibody and does not bind in a significant amount to other proteins present in the sample.

“Administration”, “therapy” and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. “Administration”, “therapy” and “treatment” can refer, e.g., to therapeutic, pharmacokinetic, diagnostic, research, and experimental methods. Treatment of a cell encompasses contact of a reagent to the cell, as well as contact of a reagent to a fluid, where the fluid is in contact with the cell. “Administration”, “therapy” and “treatment” also mean in vitro and ex vivo treatments, e.g., of a cell, by a reagent, diagnostic, binding composition, or by another cell. Within the present description of the invention the terms “in vitro” and “ex vivo” have a similar meaning and may be used interchangeably. Within the present invention the treatment of the “condition” includes any therapeutic use including prophylactic and curative uses of the anti-human APRIL antibody. Therefore the term “condition” may refer to disease states but also to physiological states in the prophylactic setting where physiology is not altered to a detrimental state.

The antibody DNA also may be modified, for example, by substituting the coding sequence for human heavy- and light-chain constant domains in place of the homologous murine sequences (U.S. Pat. No. 4,816,567; Morrison, et al., 1984, Proc. Natl Acad. Sci. USA, 81:6851), or by covalently joining to the immunoglobulin coding sequence all or part of the coding sequence for non-immunoglobulin material (e.g., protein domains). Typically such non-immunoglobulin material is substituted for the constant domains of an antibody, or is substituted for the variable domains of one antigen-combining site of an antibody to create a chimeric bivalent antibody comprising one antigen-combining site having specificity for an antigen and another antigen-combining site having specificity for a different antigen.

Amino acid sequence variants of the anti-human APRIL antibodies of the invention are prepared by introducing appropriate nucleotide changes into the coding DNAs, or by peptide synthesis. Such variants include, for example, deletions from, and/or insertions into, and/or substitutions of, residues within the amino acid sequences shown for the anti-APRIL antibodies. Any combination of deletion, insertion, and substitution is made to arrive at the final construct, provided that the final construct possesses the desired characteristics. The amino acid changes also may alter post-translational processes of the anti-APRIL antibodies, such as changing the number or position of glycosylation sites.

A useful method for identification of certain residues or regions of the anti-APRIL antibodies polypeptides that are preferred locations for mutagenesis is called “alanine scanning mutagenesis,” as described by Cunningham and Wells, 1989, Science 244: 1081-1085. Here, a residue or group of target residues are identified (e.g., charged residues such as Arg, Asp, His, Lys, and Glu) and replaced by a neutral or negatively charged amino acid (most preferably alanine or polyalanine) to affect the interaction of the amino acids with APRIL antigen. The amino acid residues demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, Ala scanning or random mutagenesis is conducted at the target codon or region and the expressed anti-APRIL antibodies' variants are screened for the desired activity.

Ordinarily, amino acid sequence variants of the anti-APRIL antibodies will have an amino acid sequence having at least 75% amino acid sequence similarity with the original antibody amino acid sequences of either the heavy or the light chain more preferably at least 80%, more preferably at least 85%, more preferably at least 90%, and most preferably at least 95%, 98% or 99%. Similarity or homology with respect to this sequence is as defined above.

Antibodies having the characteristics identified herein as being desirable can be screened for increased biologic activity in vitro or suitable binding affinity. To screen for antibodies that bind to the same epitope on human APRIL as hAPRIL.01A, a routine cross-blocking assay such as that described in Antibodies, A Laboratory Manual, Cold Spring Harbor Laboratory, Ed Harlow and David Lane (1988), can be performed. Antibodies that bind to the same epitope are likely to cross-block in such assays, but not all cross-blocking antibodies will necessarily bind at precisely the same epitope since cross-blocking may result from steric hindrance of antibody binding by antibodies bind at overlapping epitopes, or even nearby non-overlapping epitopes.

Alternatively, epitope mapping, e.g., as described in Champe et al., 1995, J. Biol. Chem. 270:1388-1394, can be performed to determine whether the antibody binds an epitope of interest. “Alanine scanning mutagenesis,” as described by Cunningham and Wells, 1989, Science 244: 1081-1085, or some other form of point mutagenesis of amino acid residues in human APRIL may also be used to determine the functional epitope for anti-APRIL antibodies of the present invention.

Another method to map the epitope of an antibody is to study binding of the antibody to synthetic linear and CLIPS peptides that can be screened using credit-card format mini PEPSCAN cards as described by Slootstra et al. (Slootstra et al., 1996, Mol. Diversity 1: 87-96) and Timmerman et al. (Timmerman et al., 2007, J. Mol. Recognit. 20: 283-299). The binding of antibodies to each peptide is determined in a PEPSCAN-based enzyme-linked immuno assay (ELISA).

Additional antibodies binding to the same epitope as hAPRIL.01A may be obtained, for example, by screening of antibodies raised against APRIL for binding to the epitope, or by immunization of an animal with a peptide comprising a fragment of human APRIL comprising the epitope sequences. Antibodies that bind to the same functional epitope might be expected to exhibit similar biological activities, such as similar APRIL binding and BCMA and TACI blocking activity, and such activities can be confirmed by functional assays of the antibodies.

The antibody can be selected from any class of immunoglobulins, including IgM, IgG, IgD, IgA, and IgE. Preferably, the antibody is an IgG antibody. Any isotype of IgG can be used, including IgG1, IgG2, IgG3, and IgG4. Variants of the IgG isotypes are also contemplated. The antibody may comprise sequences from more than one class or isotype. Optimization of the necessary constant domain sequences to generate the desired biologic activity is readily achieved by screening the antibodies using biological assays known in the art or as described herein.

Likewise, either class of light chain can be used in the compositions and methods herein. Specifically, kappa, lambda, or variants thereof are useful in the present compositions and methods.

The antibodies and antibody fragments of the invention may also be conjugated with cytotoxic payloads such as cytotoxic agents or radionucleotides such as ⁹⁹Tc, ⁹⁰Y, ¹¹¹In, ³²P, ¹⁴C, ¹²⁵I, ³H, ¹³¹I, ¹¹C, ¹⁵O, ¹³N, ¹⁸F, ³⁵S, ⁵¹Cr, ⁵⁷To, ²²⁶Ra, ⁶⁰Co, ⁵⁹Fe, ⁵⁷Se, ¹⁵²Eu, ⁶⁷Cu, ²¹⁷Ci, ²¹¹At, ²¹²Pb, ⁴⁷Sc, ¹⁰⁹Pd, ²³⁴Th, and ⁴⁰K, ¹⁵⁷Gd, ⁵⁵Mn, ⁵²Tr and ⁵⁶Fe. Such antibody conjugates may be used in immunotherapy to selectively target and kill cells expressing a target (the antigen for that antibody) on their surface. Exemplary cytotoxic agents include ricin, vinca alkaloid, methotrexate, Psuedomonas exotoxin, saporin, diphtheria toxin, cisplatin, doxorubicin, abrin toxin, gelonin and pokeweed antiviral protein.

The antibodies and antibody fragments of the invention may also be conjugated with fluorescent or chemilluminescent labels, including fluorophores such as rare earth chelates, fluorescein and its derivatives, rhodamine and its derivatives, isothiocyanate, phycoerythrin, phycocyanin, allophycocyanin, o-phthaladehyde, fluorescamine, ¹⁵²Eu, dansyl, umbelliferone, luciferin, luminal label, isoluminal label, an aromatic acridinium ester label, an imidazole label, an acridimium salt label, an oxalate ester label, an aequorin label, 2,3-dihydrophthalazinediones, biotin/avidin, spin labels and stable free radicals.

Any method known in the art for conjugating the antibody molecules or protein molecules of the invention to the various moieties may be employed, including those methods described by Hunter et al., 1962, Nature 144:945; David et al., 1974, Biochemistry 13:1014; Pain et al., 1981, J. Immunol. Meth. 40:219; and Nygren, J., 1982, Histochem. and Cytochem. 30:407. Methods for conjugating antibodies and proteins are conventional and well known in the art.

Antibody Purification

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. Carter et al., 1992, Bio/Technology 10:163-167 describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc region that is present in the antibody. Protein A can be used to purify antibodies that are based on human Ig.gamma1, Ig.gamma2, or Ig.gamma4 heavy chains (Lindmark et al., 1983, J. Immunol. Meth. 62:1-13). Protein G is recommended for all mouse isotypes and for human .gamma.3 (Guss et al., 1986, EMBO J 5:1567-1575). The matrix to which the affinity ligand is attached is most often agarose, but other matrices are available.

Mechanically stable matrices such as controlled pore glass or poly(styrenedivinyl)benzene allow for faster flow rates and shorter processing times than can be achieved with agarose. Where the antibody comprises a C_{H}3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

In one embodiment, the glycoprotein may be purified using adsorption onto a lectin substrate (e.g. a lectin affinity column) to remove fucose-containing glycoprotein from the preparation and thereby enrich for fucose-free glycoprotein.

Pharmaceutical Formulations

The invention comprises pharmaceutical formulations of an anti-human APRIL antibody. To prepare pharmaceutical or sterile compositions, the antibody, in particular an antibody or fragment thereof, is admixed with a pharmaceutically acceptable carrier or excipient, see, e.g., Remington's Pharmaceutical Sciences and U.S. Pharmacopeia: National Formulary, Mack Publishing Company, Easton, Pa. (1984). Formulations of therapeutic and diagnostic agents may be prepared by mixing with physiologically acceptable carriers, excipients, or stabilizers in the form of, e.g., lyophilized powders, slurries, aqueous solutions or suspensions (see, e.g., Hardman, et al., 2001, Goodman and Gilman's The Pharmacological Basis of Therapeutics, McGraw-Hill, New York, N.Y.; Gennaro, 2000, Remington: The Science and Practice of Pharmacy, Lippincott, Williams, and Wilkins, New York, N.Y.; Avis, et al. (eds.), 1993, Pharmaceutical Dosage Forms: Parenteral Medications, Marcel Dekker, NY; Lieberman, et al. (eds.), 1990, Pharmaceutical Dosage Forms: Tablets, Marcel Dekker, NY; Lieberman, et al. (eds.), 1990, Pharmaceutical Dosage Forms: Disperse Systems, Marcel Dekker, NY; Weiner and Kotkoskie, 2000, Excipient Toxicity and Safety, Marcel Dekker, Inc., New York, N.Y.).

Toxicity and therapeutic efficacy of the antibody compositions, administered alone or in combination with another agent, such as the usual anti-cancer drugs, can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio between LD50 and ED50. The data obtained from these cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized.

Suitable routes of administration include parenteral administration, such as intramuscular, intravenous, or subcutaneous administration and oral administration. Administration of antibodies, used in the pharmaceutical composition or to practice the method of the present invention can be carried out in a variety of conventional ways, such as oral ingestion, inhalation, topical application or cutaneous, subcutaneous, intraperitoneal, parenteral, intraarterial or intravenous injection. In one embodiment, the antibody of the invention is administered intravenously. In another embodiment, the antibody of the invention is administered subcutaneously.

Alternatively, one may administer the antibody in a local rather than systemic manner, for example, via injection of the antibody directly into the site of action, often in a depot or sustained release formulation. Furthermore, one may administer the antibody in a targeted drug delivery system.

A preferred dose protocol is one involving the maximal dose or dose frequency that achieves a desired therapeutic effect (e.g., reducing IgA levels) while avoiding significant undesirable side effects. Dosing of the antibodies as described herein can be about every week, about every two weeks, about every three weeks, about every 4 weeks, about every 8 weeks, etc., either buy intraveneous injection, or by subcutaneous injection (e.g., into the thigh, abdomen, upper arm, etc.). The dose per injection or infusion may be about 10 to 1350 mg, e.g. about 50 mg., about 150 mg, about 300 mg, about 450 mg, about 600 mg, about 750 mg, about 1000 mg, or about 1350 mg. In certain embodiments, dosing of the anti-APRIL antibody will be by subcutaneous injection, with a dose per dosing event (where a “dosing event” refers to one or more deliveries, such as injections, intended to provide a single administration to the individual, where the administrations are given in the same or different sites on the individual) of about 600 mg, with a dosing frequency of once every week, or once every two weeks. A preferred formulation for intraveneous dosing is an aqueous buffered solution at a concentration of about 15-25 mg/mL, or about 20 mg, while the preferred formulation for subcutaneous dosing is at about 125-175 mg, or about 150 mg. These formulations preferably comprise L-histidine, L-arginine, sorbitol, and polysorbate 20 at pH 6.3±0.2. Preferably the L-histidine is at a concentration of about 8-12 mM, or about 10 mM, the L-arginine is at a concentration of about 60-90 mM, or about 75 mM, the sorbitol is at a concentration of about 2.4-3.6%, or about 3% (w/w), and the polysorbate 20 is at a concentration of about 0.008-0.012%, or about 0.01% (w/w). More preferably the aqueous buffered solution comprises, consists essentially of, or consists of 10 mM L-histidine, 75 mM L-arginine, 3% (w/w) sorbitol and 0.01% (w/w) polysorbate 20 at pH 6.3±0.2. The pH of the aqueous buffered solution can be adjusted to 6.3±0.2 using a suitable sterile acid/base, such as hydrochloric acid and sodium hydroxide. Formulations for intraveneous infusion can be diluted in sterile saline (0.9%) prior to infusion, for example the desired amount of anti-APRIL antibody can be diluted to a volume of about 250 mL, for example 15 mL of a 20 mg/mL formulation of antibody can be diluted with 235 mL of sterile saline solution prior to infusion of a 300 mg dose. The formulation for subcutaneous injection can be used without further dilution.

The therapeutically effective amount and the frequency of administration of, and the length of treatment with, an anti-APRIL antibody disclosed herein to treat an antibody-associated condition may depend on various factors, including the nature and severity of the condition, the potency of the antibody, the mode of administration, the age, body weight, general health, gender and diet of the subject, and the response of the subject to the treatment, and can be determined by the treating physician. The anti-APRIL antibody can be administered once daily, once every 2 days, once every 3 days, twice weekly, once weekly, once every 2 weeks, once every 3 weeks, once monthly, once every 6 weeks, once every 2 months or once every 3 months, or as deemed appropriate by the treating physician.

The anti-APRIL antibody can be administered over a period of at least about 1 week, 2 weeks, 1 month (4 weeks), 6 weeks, 2 months, 3 months, 6 months, 1 year, 2 years, 3 years or longer, or as deemed appropriate by the treating physician. The APRIL-associated condition can be a chronic condition. A chronic condition can exist for, e.g., at least about 6 weeks, 2 months, a year, or longer. The antibody can be administered over a period of at least about 6 weeks, 2 months, 3 months or 6 months, a year, or even multiple years as required for medical care of an individual.

An anti-APRIL antibody can also be administered in an irregular manner to treat an antibody-associated condition. Early achievement of an effective target antibody concentration (a therapeutic dose level) with a loading dose followed by maintenance dosing with the antibody (frontloading) may be more effective than conventional therapy in terms of requiring a lower total antibody dose and faster time to maximum target engagement. As used herein, such an administration protocol is referred to as a “loading/maintenance administration protocol.” An effective target antibody concentration may be reached in 4 weeks or less, preferably 3 weeks or less, more preferably 2 weeks or less, most preferably 1 week or less, including 1 day or less using a loading dose. The target serum concentration is then maintained by administration of an equal or smaller (or less frequent) maintenance dose during the remainder of the treatment regimen or until suppression of disease symptoms is achieved.

The term “frontloading” when referring to drug administration refers to the initial loading dose, followed by the maintenance dose. The initial loading dose (single or multiple) is intended to more quickly increase the serum drug concentration of an animal or human patient to an effective target serum concentration. In various embodiments, frontloading is accomplished by initial dosing delivered over 3 weeks or less so that the antibody reaches the target serum concentration. Preferably, the loading dose or series of doses is administered for 2 weeks or less, more preferably 1 week or less, e.g. 1 day or less. Most preferably, the loading dosing is a single dosing, with no maintenance dosing thereafter for at least one week, and the loading dosing is administered in 1 day or less. In order to avoid adverse immune reactions to antibody drugs, it may be preferred to deliver the loading dose of antibody is administered by intravenous injection. The present invention includes loading and maintenance doses of frontloading drug delivery by intravenous or subcutaneous administration.

Administration of the loading dose can be, for example, one or more dosings at a time interval of at least about 1, 2, 3, 4, 5, 6, 7 or 8 weeks apart. In some embodiments, the at least one loading dose is administered by one or more intravenous injections and then at least one maintenance dose by one or more intravenous or subcutaneous administrations. In other embodiments, the instructions can be for administering at least one loading dose by, for example, one or more intravenous or subcutaneous administrations and at least one maintenance dose by one or more intravenous or subcutaneous administrations. In certain embodiments, both the at least one loading dose as well as the at least one maintenance dose is administered subcutaneously. In other embodiments, the at least one loading dose is administered by intravenous infusion followed by at least one maintenance dose administered subcutaneously. For example, the method of treatment can comprise administering a loading dose of 150-1350 mg of the anti-APRIL antibody by intravenous infusion or subcutaneous injection. After the loading dose (e.g. 1 week, 2 weeks, 3 weeks or 4 weeks after the loading dose), a maintenance dose of 600 mg or less of the anti-APRIL antibody can be administered every 4 weeks or less, preferably every 3 weeks or less, more preferably every 2 weeks or less, and in embodiments every 1 week or less, by subcutaneous injection. The choice of loading and maintenance dosages and intervals can be made according to the ability of the animal or human patient to tolerate administration of the antibody to the body and according to a desired serum level of APRIL to achieve.

A loading dose of a drug can be larger (e.g., about 1.5, 2, 3, 4 or 5 times larger) than a subsequent maintenance dose. The one or more therapeutically effective maintenance doses can be any therapeutically effective amount described herein. The loading dose can be about 2 or 3 times larger than the maintenance dose. The anti-APRIL antibody can be administered in two (or more) loading doses prior to the maintenance dose. A first loading dose of the antibody or fragment thereof can be administered on day 1, a second loading dose can be administered, e.g., about 1 or 2 weeks later, and a maintenance dose can be administered, e.g., once weekly or once every 2 weeks thereafter for the duration of treatment. The first loading dose can be about 3 or 4 times larger than the maintenance dose, and the second loading dose can be about 2, 3, 4, 5, or more times larger than the maintenance dose.

As used herein, “inhibit” or “treat” or “treatment” includes a postponement of development of the symptoms associated with disease and/or a reduction in the severity of such symptoms that will or are expected to develop with said disease. The terms further include ameliorating existing symptoms, preventing additional symptoms, and ameliorating or preventing the underlying causes of such symptoms. Thus, the terms denote that a beneficial result has been conferred on a vertebrate subject with a disease.

The antibody of the present invention for therapeutic purposes is administered in a therapeutically effective amount. As used herein, the term “therapeutically effective amount” or “effective amount” refers to an amount of an anti-APRIL antibody or fragment thereof, that when administered alone or in combination with an additional therapeutic agent to a cell, tissue, or subject is effective to prevent or ameliorate the disease or condition to be treated. A therapeutically effective dose further refers to that amount of the compound sufficient to result in amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously. An effective amount of therapeutic will decrease the symptoms typically by at least 10%; usually by at least 20%; preferably at least about 30%; more preferably at least 40%, and most preferably by at least 50%.

Methods for co-administration or treatment with a second therapeutic agent are well known in the art, see, e.g., Hardman, et al. (eds.), 2001, Goodman and Gilman's The Pharmacological Basis of Therapeutics, 10th ed., McGraw-Hill, New York, N.Y.; Poole and Peterson (eds.), 2001, Pharmacotherapeutics for Advanced Practice: A Practical Approach, Lippincott, Williams & Wilkins, Phila., Pa.; Chabner and Longo (eds.), 2001, Cancer Chemotherapy and Biotherapy, Lippincott, Williams & Wilkins, Phila., Pa.

The pharmaceutical composition of the invention may also contain other agents, including but not limited to a cytotoxic, chemotherapeutic, cytostatic, anti-angiogenic or antimetabolite agents, a tumor targeted agent, an immune stimulating or immune modulating agent or an antibody conjugated to a cytotoxic, cytostatic, or otherwise toxic agent. The pharmaceutical composition can also be employed with other therapeutic modalities such as surgery, chemotherapy and radiation.

### PREFERRED EMBODIMENTS

The following are preferred embodiments of the invention:

1. An antibody formulation suitable for pharmaceutical infusion or subcutaneous injection, comprising:
an anti-APRIL antibody at a concentration of between about 20 mg/mL to about 190 mg/mL;
about 10 mM L-histidine;
about 75 mM L-arginine;
about 3% wt % sorbitol;
about 0.01 wt % polysorbate 20; and
a pH of about 6.0 to about 6.6;
wherein the formulation (i) has a viscosity of about 16 cP or less, (ii) does not comprise glutamic acid or its salt, (iii) has an osmolality of between about 250 mOsm/kg to about 390 mOsm/kg, and (iv) has an OD330 of less than about 1.0.
   2. An antibody formulation according to embodiment 1, wherein the formulation maintains at least 96% purity of the anti-APRIL antibody following storage at 2-8° C. for 9 months following manufacture of the formulation.
   3. An antibody formulation according to embodiment 2, wherein the formulation maintains at least 95% purity of the anti-APRIL antibody following storage at 25° C. for 6 months following manufacture of the formulation.
   4. An antibody formulation according to one of embodiments 1-3, wherein the formulation has a second virial coefficient of 2.5×10⁻⁵ mol·mL/g² or greater measured at 25° C.
   5. An antibody formulation according to one of embodiments 1-4, wherein the formulation has a calculated isoelectric point of about 7.4 or greater.
   6. An antibody formulation according to one of embodiments 1-5, wherein the anti-APRIL antibody in the formulation is at a concentration of about 150 mg/mL.
   7. An antibody formulation according to embodiment 6, wherein the formulation has an osmolality of between about 290 mOsm/kg to about 390 mOsm/kg.
   8. An antibody formulation according to one of embodiments 6 or 7, wherein the formulation has an OD330 of about 0.8 or less.
   9. An antibody formulation according to one of embodiments 1-5, wherein the anti-APRIL antibody is at a concentration of about 20 mg/mL.
   10. An antibody formulation according to embodiment 9, wherein the formulation has an osmolality of between about 293 mOsm/kg to about 333 mOsm/kg.
   11. An antibody formulation according to one of embodiments 1-10, wherein the anti-APRIL antibody is a humanized antibody comprising a heavy chain variable region.light chain variable region pair selected from the group consisting of VH11.VL15, VH12.VL15, VH13.VL15, VH14.VL15, VH14_1.VL15, VH14_1C.VL15, VH14_1D.VL15, VH14_1E.VL15, and VH14_1G.VL15.
   12. An antibody formulation according to one of embodiments 1-11, wherein the formulation is free of glycine, carbonate, HEPES, phosphate, citrate, and acetate.
   13. A single-use or multi-use vial comprising the antibody formulation of one of embodiments 1-12.
   14. The single-use or multi-use vial of embodiment 13 containing a volume of between 0.5 mL and 50 mL of the formulation having an anti-APRIL antibody concentration of between about 20 mg/mL to about 190 mg/mL.
   15. The single-use or multi-use vial of embodiment 14, wherein the formulation has an anti-APRIL antibody concentration of about 20 mg/mL.
   16. The single-use or multi-use vial of embodiment 14, wherein the formulation has an anti-APRIL antibody concentration of about 150 mg/mL.
   17. The single-use or multi-use vial of one of embodiments 14-16, containing a volume of 5 mL of the formulation.
   18. A pre-filled syringe, autoinjector, or injector pen comprising the antibody formulation of one of embodiments 1-12.
   19. The pre-filled syringe, autoinjector, or injector pen of embodiment 18 containing a volume of between about 0.5 mL and about 10 mL of the formulation having an anti-APRIL antibody concentration of between about 20 mg/mL to about 190 mg/mL.
   20. The pre-filled syringe, autoinjector, or injector pen of embodiment 19, wherein the formulation has an anti-APRIL antibody concentration of about 20 mg/mL.
   21. The pre-filled syringe, autoinjector, or injector pen of embodiment 19, wherein the formulation has an anti-APRIL antibody concentration of about 150 mg/mL.
   22. The pre-filled syringe, autoinjector, or injector pen of one of embodiments 19-21, containing a volume of about 2 mL of the formulation.
   23. A method of administering an anti-APRIL antibody to an individual in need thereof comprising administering the formulation of one of embodiments 1-12 by subcutaneous injection into the individual.
   24. A method according to embodiment 23, wherein the method comprises repeating the administration on at least an every week (QW) schedule for at least 2 administration cycles.
   25. A method according to embodiment 23, wherein the method comprises repeating the administration on at least an every two weeks (Q2W) schedule for at least 2 administration cycles.
   26. A method according to embodiment 23, wherein the method comprises repeating the administration on at least an every 4 weeks (Q4W) or monthly (QMT) schedule for at least 2 administration cycles.
   27. A method according to one of embodiments 23-26, wherein a total dose of between about 10 mg to about 1350 mg of the anti-APRIL antibody is administered per dosing event.
   28. A method according to embodiment 27, wherein about 2 mL of the formulation at a concentration of about 150 mg/mL of the anti-APRIL antibody is delivered per administration,
   29. A method according to embodiment 27, wherein about 4 mL of the formulation at a concentration of about 150 mg/mL of the anti-APRIL antibody is delivered per administration, and each dosing event comprises one or more subcutaneous injections.
   30. A method according to one of embodiments 23-29, wherein the formulation is subcutaneously administered into a site in the individual's thigh, abdomen, or upper arm.
   31. A method of administering an anti-APRIL antibody to an individual in need thereof comprising administering the formulation of one of embodiments 1-12 by intravenous infusion into the individual.
   32. A method according to embodiment 31, wherein the method comprises repeating the administration on at least a QW schedule for at least 2 administration cycles.
   33. A method according to embodiment 31, wherein the method comprises repeating the administration on at least a Q2W schedule for at least 2 administration cycles.
   34. A method according to embodiment 31, wherein the method comprises repeating the administration on at least a Q4W or monthly schedule for at least 2 administration cycles.
   35. A method according to any one of embodiments 31-34, wherein the intravenous infusion comprises:
   diluting the formulation of one of embodiments 1-12 to a concentration of between about 0.1 mg/mL to about 10 mg/mL in 0.9% saline; and
   administering a total dose of between about 10 mg to about 1350 mg of the anti-APRIL antibody to the individual in a single intravenous dose of the diluted formulation over a period of about 2 hours.
   36. A method according to embodiment 35, wherein 15 mL of the formulation at a concentration of 20 mg/mL is added to 235 mL of 0.9% saline to provide the intraveneous dose at a concentration of 1.2 mg/mL.
   37. A method of administering an anti-APRIL antibody to an individual in need thereof comprising administering an anti-APRIL by a loading/maintenance administration protocol.
   38. A method according to embodiment 37, wherein the loading component of the loading/maintenance administration protocol comprises one or more administrations of the anti-APRIL antibody at a higher concentration than the anti-APRIL antibody concentration in the maintenance component of the loading/maintenance administration protocol.
   39. A method according to embodiment 37, wherein the loading component of the loading/maintenance administration protocol comprises one or more administrations of the anti-APRIL antibody at a higher frequency than the frequency of administration of the anti-APRIL antibody in the maintenance component of the loading/maintenance administration protocol.
   40. A method according to one of embodiments 37-39, wherein the loading component of the loading/maintenance administration protocol comprises one or more administrations of the anti-APRIL antibody at a different route than the route of administration of the anti-APRIL antibody in the maintenance component of the loading/maintenance administration protocol.
   41. A method according to embodiment 37, wherein the loading component of the loading/maintenance administration protocol comprises one or more intravenous administrations of the anti-APRIL antibody and the maintenance component of the loading/maintenance administration protocol comprises one or more subcutaneous administrations of the anti-APRIL antibody.
   42. A method according to one of embodiments 37-41, wherein the anti-APRIL antibody is an antibody formulation according to one of embodiments 1-12.
   43. A method according to one of embodiments 23-41, wherein the individual in need thereof has a serum IgA level greater than 4 g/L.
   44. A method according to one of embodiments 23-41, wherein the individual in need thereof is an IgA nephropathy patient.
   45. A method according to one of embodiments 23-41, wherein the individual has a hyperimmunoglobulinemia.

The invention will now be further illustrated and supported with reference to the following non-limiting experiments.

### EXAMPLES

### Example 1

The following examples describe the development of high-dose anti-APRIL formulation with a viscosity lower than 16 cP. Formulations were subjected to temperature stress (−70° C., 2-8° C., 25° C. and 45° C.), freeze/thaw cycling, and gentle shaking with the intention of selecting a formulation most suitable for the bulk drug substance and filled drug product at the desired concentration. The predicted SE- and CEX-UPLC stability at 5° C. for the formulation was approximately 2 and 6 years, respectively. Samples were characterized for the following:
Visual Appearance (C=clear; O=opalescent; NC=no color; Y=light yellow; NP=no visible particulates)
Concentration
Turbidity by A330 nm
Size Exclusion Ultra High Performance Chromatography (SE-UPLC)
Cation Exchange UPLC (CEX-UPLC)
Dynamic light scattering/static light scattering
pH
Osmolality
Viscosity

Reagents:

Anti-APRIL antibody: VH14_1G.VL15

| | | |
|---|---|---|
| Histidine | J.T. Baker | 2080-06 |
| Arginine | J.T. Baker | 2066-06 |
| Glutamic Acid | J.T. Baker | 2077.06 |
| Sorbitol | EMD Millipore | 1.03583.2503 |
| Sodium Chloride | J.T. Baker | 7647-145 |
| Polysorbate-20 | J.T. Baker | 4116.04 |
| Piperazine | Sigma | 80621 |
| Imidazole | Fluka | 56749 |
| Tris | Sigma | 252859 |
| Disodium hydrogen phosphate | J.T. Baker | 8327-01 |
| anhydrous | | |
| Potassium dihydrogen phosphate | Millipore | 7778-77-0 |

A total of 11 formulations (see Table 2), with six formulations at a target concentration of >150 mg/mL and five formulations at a concentration of 200 mg/mL were prepared. All formulations were prepared with histidine buffer at pH 6.1 and 6.3.

TABLE 2
List of formulations

| | | | | | Glutamic | | | |
|---|---|---|---|---|---|---|---|---|
| | Formulation | | Conc. | Arginine | Acid | Sorbitol | NaCl | 0.01% |
| | ID | pH | (mg/mL) | (mM) | (mM) | (%) | (mM) | PS20 |
| 10 mM | 1 | 6.1 | >150 | 150 | 150 | − | − | + |
| Histidine | 1 | 6.1 | 200 | 150 | 150 | − | − | + |
| | 2 | 6.1 | >150 | 55 | 55 | 3 | − | + |
| | 2 | 6.1 | 200 | 55 | 55 | 3 | − | + |
| | 3 | 6.3 | >150 | 55 | 55 | 3 | − | + |
| | 3 | 6.3 | 200 | 55 | 55 | 3 | − | + |
| | 4 | 6.1 | >150 | 55 | 55 | − | 80 | + |
| | 5 | 6.3 | >150 | 55 | 55 | − | 80 | + |
| | 6 | 6.3 | 200 | 150 | 150 | − | − | + |

| | | | | |
|---|---|---|---|---|
| Control | 11 | 6.3 | >150 | 10 mM histidine, 3% sorbitol, 75 mM |
| | | | | arginine, 0.01% PS20, pH 6.3 |
| | 11 | 6.3 | 200 | 10 mM histidine, 3% sorbitol, 75 mM |
| | | | | arginine, 0.01% PS20, pH 6.3 |

Anti-APRIL antibody was concentrated to 50 mg/mL by tangential flow filtration (TFF) at a maximum flow rate of 40 mL/min. Concentrated material was then retrieved and centrifuged 5 minutes at 8500 rcf, and filtered through a 0.22 μm PVDF membrane. Pre-hydrated 10 kDa MWCO dialysis cassettes were filled with material and dialyzed at 2-8° C. against 200 mL of buffer (3 changes of 200 mL). Total dialysis time was two days. Recovered protein samples were then spin-concentrated at 3750 rcf in 10 kDa MWCO centrifugal filter units over the course of several hours to reach the target values. The concentrated formulations were either analyzed as over-concentrated samples (e.g. >150 mg/mL) or diluted to a specific target concentration (e.g. 200 mg/mL) using the corresponding buffers. Polysorbate-20 (PS20), prepared in the corresponding buffer was added to each formulation to a 0.01% (w/v) final concentration.

The recovery calculations and the observations for visual appearance are shown in the following Table 3. After the TFF step, a recovery of 98.2% was calculated from protein content. A white, turbid solution was observed. Thus centrifugation and filtration of this protein solution was carried out before the dialysis step to remove the particles. The filtration step had little to no effect on protein concentration. During the spin concentration step, target concentrations of >150 mg/mL were reached for all the prospective formulations evaluated in this screening.

TABLE 3
Protein concentration, recovery and visual appearance of material
during the concentration step:

| | Volume | Conc. | Material | Recovery | Visual |
|---|---|---|---|---|---|
| | (mL) | (mg/mL) | (mg) | (%) | appearance |
| Starting material | 315 | 3.21 | 1011.2 | N.A. | Clear, No color |
| Post-TFF | 20.5 | 48.5 | 993.8 | 98.2 | Turbid, White solution |
| Post-TFF, filtered | 20.5 | 48.5 | 993.8 | 100% | Clear, No color |
| Pre-dialysis | 2.06 | 48.5 | 99.9 | N.A. | Clear, No color |
| Formulation 1 Post-Spinconc. | 0.401 | 190.6 | 76.4 | 76.5 | Clear, Light yellow |
| Formulation 2 Post-Spinconc. | 0.446 | 185.9 | 82.9 | 83.0 | Clear, Light yellow |
| Formulation 3 Post-Spinconc. | 0.472 | 177.8 | 83.9 | 84.0 | Clear, Light yellow |
| Formulation 4 Post-Spinconc. | 0.452 | 155.2 | 70.2 | 70.2 | Clear, Light yellow |
| Formulation 5 Post-Spinconc. | 0.446 | 178.5 | 79.6 | 79.7 | Clear, Light yellow |
| Formulation 11 Post-Spinconc. | 0.480 | 181.2 | 87.0 | 87.1 | Clear, Light yellow |

The following Table 4 summarizes the analytics obtained for this study. At this step, the % recovery ranged from 70.2-87.1% when compared to the initial amount of material loaded in the cassette. Viscosities ranged from 7.7 to 15.4 cP, with formulation 1 being the highest concentration with low viscosity. The comparison of formulations 3 and 11 suggest that in the presence of sorbitol, the combination of arginine with glutamic acid yield in lower viscosities than arginine alone. Overall, this first screening experiment suggest that salt should be avoided to keep viscosities low. The comparison of formulation 3 and 5 highlight this finding. Overall, the colloidal stability is acceptable for all tested formulations, with A2 values being positive and within similar ranges.

TABLE 4
Composition, viscosity, osmolality and second virial coefficient (A2) obtained during the formulation screen:

| | | | | Glutamic | | | | | | | A2 |
|---|---|---|---|---|---|---|---|---|---|---|---|
| | | | Arginine | Acid | Sorbitol | NaCl | 0.01% | Conc. | Viscosity | Osmolality | (mol mL/g²) |
| | ID | pH | (mM) | (mM) | (%) | (mM) | PS20 | (mg/mL) | (cP) | (mOsm/kg) | × 10⁻⁵ |
| 10 mM Histidine | # 1 | 6.1 | 150 | 150 | − | − | + | 190.6 | 12.1 | 288 | 3.315 |
| | # 2 | 6.1 | 55 | 55 | 3 | − | + | 185.9 | 12.9 | 274 | 4.089 |
| | # 3 | 6.3 | 55 | 55 | 3 | − | + | 177.8 | 10.5 | 298 | 3.670 |
| | # 4 | 6.1 | 55 | 55 | − | 80 | + | 155.2 | 7.7 | 246 | 1.293 |
| | # 5 | 6.3 | 55 | 55 | − | 80 | + | 178.5 | 14.2 | 243 | 2.980 |
| | # 11 | 6.3 | 75 | − | 3 | − | + | 181.2 | 15.4 | 308 | 2.792 |
| | ctrl | | | | | | | | | | |

### Example 2

To assess the feasibility of obtaining protein concentration >200 mg/mL, fresh samples were prepared from the starting anti-APRIL antibody. Formulations containing sodium chloride were eliminated as salts were detrimental to keeping viscosities to lower values. Samples were overconcentrated to 239-252 mg/mL during the spin-concentration step. Of note, recovery data, as calculated from protein quantitation by A280, were lower than previously observed, with values ranging from 42.7-69.4%. The samples were then diluted to 200 mg/mL with the corresponding formulation buffer, while adjusting the PS20 content to a final concentration of 0.01%. Viscosity measurements were high, with values ranging from 26.2 cP to 47.0 cP. Of note, viscosity measurements taken in samples concentrated at 155-190 mg/mL are much lower than the viscosity observed in samples at 200 mg/mL. This increase in viscosity values suggest that some sort of deleterious protein-protein interaction occurring during the extreme protein over-concentration. Although centrifugal devices allow for the concentration of small sample volumes, test samples do experience high transmembrane pressure created by g-force during the centrifugation process which could be an additional cause for protein denaturation. Results are provided in the following Tables 5 and 6.

TABLE 5
Protein concentration, recovery and visual appearance of material during the
concentration step:

| | Volume | Conc. | Material | Recovery | |
|---|---|---|---|---|---|
| | (mL) | (mg/mL) | (mg) | (%) | Visual appearance |
| Starting material | 340.0 | 3.34 | 1135.6 | N.A. | Clear, no color, No particles |
| Post-TFF | 28.0 | 39.5 | 1106.0 | 97.4 | Turbid, White solution |
| Pre-dialysis | 4.5 | 39.5 | 177.8 | N.A. | Clear, no color, No particles |
| Formulation 1 Post-SpinCon | 0.312 | 244.7 | 76.2 | 42.9 | Clear, light yellow, No |
| | | | | | particles |
| Formulation 2 Post-SpinCon | 0.302 | 252.1 | 76.0 | 42.7 | Clear, light yellow, No |
| | | | | | particles |
| Formulation 3 Post-SpinCon | 0.513 | 240.8 | 123.4 | 69.4 | Clear, light yellow, No |
| | | | | | particles |
| Formulation 6 Post-SpinCon | 0.382 | 239.2 | 91.4 | 51.4 | Clear, light yellow, No |
| | | | | | particles |
| Formulation 11 Post-SpinCon | 0.347 | 251.9 | 87.3 | 49.1 | Clear, light yellow, No |
| | | | | | particles |

TABLE 6
Composition and viscosity of formulations at 200 mg/mL:

| | | | Arginine | Glutamic | Sorbitol | NaCl | 0.01% | Conc. | Viscosity |
|---|---|---|---|---|---|---|---|---|---|
| | ID | pH | (mM) | Acid (mM) | (%) | (mM) | PS20 | (mg/mL) | (cP) |
| 10 mM Histidine | # 1 | 6.1 | 150 | 150 | − | − | + | 200 | 47.0 |
| | # 2 | 6.1 | 55 | 55 | 3 | − | + | 200 | 38.2 |
| | # 3 | 6.3 | 55 | 55 | 3 | − | + | 200 | 29.5 |
| | # 6 | 6.3 | 150 | 150 | − | − | + | 200 | 33.9 |
| | # 11 | 6.3 | 75 | − | 3 | − | + | 200 | 26.2 |
| | Ctrl | | | | | | | | |

To closely monitor the sample viscosity between 190 mg/mL and 200 mg/mL and to assess the effect of the surfactant on the viscosity, a third set of fresh samples was prepared from starting anti-APRIL antibody. In this setup, formulation 1 and formulation 6 were prepared. The protein was brought to 28.8 mg/mL by TFF, followed by the dialysis of 12 mL of post-TFF material. During the spin-concentration process, a qualitative increase in viscosity was observed at concentration ˜180 mg/mL and the final concentration achieved was ˜200 mg/mL. Half of the samples were kept at the final concentration, while the other half was brought to 190 mg/mL with the corresponding formulation buffer without PS20. Viscosity was measured at both concentrations and results are presented in the following Table 7. High viscosities were observed in samples at concentration of >200 mg/mL, with values of 46.9 cP and 33.0 cP in formulation 1 and 6, respectively. When the sample concentration was brought to 190 mg/mL by dilution, the viscosities lowered to 34.2 cP and 22.5 cP. The preparation of a sample with 0.01% PS20 resulted in a viscosity of 20 cP for formulation 6.

TABLE 7
Composition and viscosity of protein solution at 190 and 200 mg/mL

| | | | | Glutamic | 0.01% | Conc. | Viscosity |
|---|---|---|---|---|---|---|---|
| | ID | pH | Arginine (mM) | Acid (mM) | PS20 | (mg/mL) | (cP) |
| 10 mM Histidine | #1 | 6.1 | 150 | 150 | − | 204.3 | 46.9 |
| | #1 | 6.1 | 150 | 150 | − | 190.0 | 34.2 |
| | #6 | 6.3 | 150 | 150 | − | 201.9 | 33.0 |
| | #6 | 6.3 | 150 | 150 | − | 190.0 | 22.5 |
| | #6 | 6.3 | 150 | 150 | + | 190.0 | 20.0 |

### Example 3

A total of four 150 mg/mL anti-APRIL (VH14_1G.VL15) antibody formulations (Table 8 below) were prepared containing histidine buffer at pH 6.1 and 6.3. A volume of 0.6 mL of test sample was aseptically placed in 2 mL glass vials that were subsequently stoppered. Stability test conditions and assays used are provided in Table 9.

TABLE 8
List of formulations tested in the study

| | Protein | | | | | | |
|---|---|---|---|---|---|---|---|
| | Concentration | | 10 mM | Arginine | Glutamic | 3% | 0.01% |
| Formulation | (mg/mL) | pH | Histidine | (mM) | acid (mM) | Sorbitol | PS20 |
| 1 | 150 | 6.1 | + | 150 | 150 | − | + |
| 3 | 150 | 6.3 | + | 55 | 55 | + | + |
| 6 | 150 | 6.3 | + | 150 | 150 | − | + |
| 11 | 150 | 6.3 | + | 75 | − | + | + |

TABLE 9
Stability test conditions and assays used in the evaluation

| | | | | | | Visual | | | | | | |
|---|---|---|---|---|---|---|---|---|---|---|---|---|
| | Sampling | | Analysis | A280 | A330 | appearance | CEX- | | SE- | | | SLS/ |
| Condition | timepoint | | batch | nm | nm | (liq) | UPLC | Viscosity | UPLC | Osmolality | pH | DLS |
| −70 | 0 | | 1 | 1 | 1 | 1 | 1 | 1 | 1 | 1 | 1 | 1 |
| −70 | 6 | w | 5 | 1 | 1 | 1 | 1 | | 1 | | 1 | |
| −70 | 12 | w | 6 | 1 | 1 | 1 | 1 | | 1 | | 1 | 1 |
| (−70° C.) | 1 | w | 3 | 1 | 1 | 1 | 1 | | 1 | | 1 | 1 |
| Freeze/thaw | | | | | | | | | | | | |
| 5 cycles | | | | | | | | | | | | |
| 4° C.- | 3 | d | 2 | 1 | 1 | 1 | 1 | | 1 | | 1 | 1 |
| 3 day shake | | | | | | | | | | | | |
| 4° C. | 1 | w | 3 | 1 | 1 | 1 | 1 | | 1 | | 1 | 1 |
| 4° C. | 3 | w | 4 | 1 | 1 | 1 | 1 | | 1 | | 1 | |
| 4° C. | 6 | w | 5 | 1 | 1 | 1 | 1 | | 1 | | 1 | |
| 4° C. | 12 | w | 6 | 1 | 1 | 1 | 1 | | 1 | | 1 | 1 |
| 25° C. | 1 | w | 3 | 1 | 1 | 1 | 1 | | 1 | | 1 | |
| 25° C. | 3 | w | 4 | 1 | 1 | 1 | 1 | | 1 | | 1 | 1 |
| 25° C. | 6 | w | 5 | 1 | 1 | 1 | 1 | | 1 | | 1 | |
| 25° C. | 12 | w | 6 | 1 | 1 | 1 | 1 | | 1 | | 1 | 1 |
| 45° C. | 1 | w | 3 | 1 | 1 | 1 | 1 | | 1 | | 1 | |
| 45° C. | 3 | w | 4 | 1 | 1 | 1 | 1 | | 1 | | 1 | 1 |

Thawed anti-APRIL antibody was centrifuged 20 minutes at 3000 rcf, then filtered on a 0.22 μm membrane to clear the few particles observed. The filtered material was concentrated by TFF over two days to reach a final concentration of 37.9 mg/mL. The concentrated material was then placed into pre-hydrate 10 kDa MWCO 70 mL capacity dialysis cassettes and dialyzed at 2-8° C. against 2 L buffer (3 changes of 2 L) over a period of two days. Recovered protein samples were then spin-concentrated at 2300 rcf in Amicon Ultra-15 tubes (Regenerated Cellulose 10 kDa MWCO) at 15° C. over the course of several hours to reach a target concentration of >150 mg/mL. The material to concentrate was split in half and concentrated on two spin concentrator to account for the large volume to process. The halves were pooled shortly after the concentration step once the target volume was reached, prior to analysis. Overconcentrated protein solutions were set aside for viscosity testing, prior to addition of surfactant. The concentrated formulations were diluted to a target of 150 mg/mL using the corresponding buffers. PS20, prepared in the corresponding buffer, was added to the formulations to a 0.01% (w/v) final concentration while targeting 150 mg/mL VH14_1 G.VL15. Under aseptic conditions, VH14_1 G.VL15 formulations were filtered through a 0.22 μm PVDF membrane and then were dispensed into depyrogenated borosilicate vials, stoppered and crimped with an aluminum seal. Vials were then placed under stress conditions for stability testing according to Table 9. At each requisite time point, samples were pulled from the condition, and visual appearance was performed in the original vials. Osmolality, viscosity, A330, pH and DLS assays were performed on undiluted samples, while A280, SE-UPLC and CEX-UPLC were performed on diluted samples, according to respective method specifications. Osmolality and viscosity were performed only at T=0 time point. Any remaining sample volume was retained at −70° C. for backup testing.

Following the various applied stress conditions, visual appearance, sample turbidity via A330, pH, and concentration was determined. The concentration of the sample was determined by absorbance measurement at 280 nm using an extinction coefficient of 1.0 mg/mL=1.4 AU. The reported, final protein concentration was corrected for scattering ((A280-A330)/ε280=Corrected Protein Concentration). Sample viscosity values were determined for in-process overconcentrated samples (prior to addition of PS20) as well as for T=0 samples. Osmolality values were determined for T=0 samples only.

During the sample preparation step, aliquots of overconcentrated samples were set aside for viscosity measurements. Viscosities ranged from 12.5 to 15.6 cP. Of note, a difference of viscosity was observed for samples at ˜190 mg/mL in the formulation screen and the sample preparation for the 12 week formulation study. This difference could be due to the difference in the volume of the processed samples during the spin concentration step, or due to the different starting material used in this study. Target antibody concentrations of 150 mg/mL were achieved for all test formulations during a dilution step while introducing surfactant to a final concentration of 0.01% PS20. The resulting 150 mg/mL formulations displayed viscosities between 6.3 and 7.8 cP and osmolalities between 324 and 358 mOsm/kg. Of important note, review of the preparation information indicated that the buffer corresponding to formulation 11 was prepared at pH 6.1 and not the target pH 6.3, thus explaining the disparity with the target pH for this formulation. The measured pH values remained within 0.1 units of the starting values for all observed time-points.

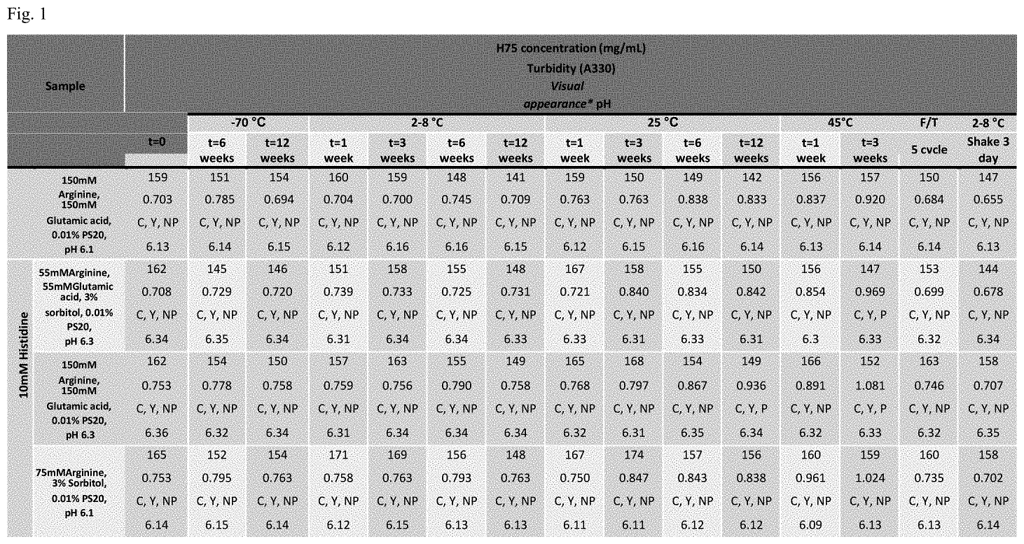

Following stress incubation, the sample visual appearance, protein concentration, turbidity, and pH were noted (FIG. 1). No clear differences were observable in regards to pH following the different stress conditions. Visual appearance remained clear, light yellowish and with no visible particulates for all formulations following the different stress conditions except for the appearance of a few particles following 12-weeks of incubation at 25° C. (formulation 6) and 3 weeks of incubation at 45° C. (formulation 3 and 6). Turbidity values were generally lower in formulation 1 and formulation 3. No discernable analytical changes in any of the formulations were apparent following 5 freeze/thaw cycles or 3-days shaking at 2-8° C.

Target antibody concentrations of 150 mg/mL were approximated in all test formulations. Sample pH values were within 0.1 units of the dialysis buffer. Note, formulation 11 was prepared at pH 6.14 rather than the target pH 6.30. Visual appearance remained clear, light yellow for all the formulations following the different stress conditions but a few particulates were observed following 12-weeks of incubation at 25° C. (formulation 6) and 3 weeks of incubation at 45° C. (formulations 3 and 6). The turbidity, as measured from A330, was generally lower in formulations 1 and 3. Results are provided in the following Tables 10 and 11.

TABLE 10
Composition and viscosity of samples

| | Protein | | | | Glutamic | | | |
|---|---|---|---|---|---|---|---|---|
| Form. | Conc. | | 10 mM | Arginine | Acid | 3% | 0.01% | Viscosity |
| ID | (mg/mL) | pH | Histidine | (mM) | (mM) | Sorbitol | PS20 | (cP) |
| 1 | 197 | 6.1 | + | 150 | 150 | − | − | 15.6 |
| 3 | 189 | 6.3 | + | 55 | 55 | + | − | 14.1 |
| 6 | 189 | 6.3 | + | 150 | 150 | − | − | 15.8 |
| 11 | 179 | 6.3 | + | 75 | − | + | − | 12.5 |

TABLE 11
pH, Osmolality, Viscosity, A330 at T = 0

| | Target | | | | Glut. | | | | Actual | | | |
|---|---|---|---|---|---|---|---|---|---|---|---|---|
| Form. | Conc. | Target | 10 mM | Arg. | acid | 3% | 0.01% | | Conc. | Osmolality | Visc. | |
| ID | (mg/mL) | pH | Hist. | (mM) | (mm) | Sorbitol | PS20 | pH | (mg/mL) | (mOsm/kg) | (cP) | A330 |
| 1 | 150 | 6.1 | + | 150 | 150 | − | + | 6.13 | 159 | 329 | 6.5 | 0.703 |
| 3 | 150 | 6.3 | + | 55 | 55 | + | + | 6.34 | 162 | 324 | 6.3 | 0.708 |
| 6 | 150 | 6.3 | + | 150 | 150 | − | + | 6.36 | 162 | 346 | 7.8 | 0.753 |
| 11 | 150 | 6.3 | + | 75 | − | + | + | 6.14 | 165 | 352 | 7.6 | 0.753 |

### Example 4

Size exclusion chromatography (also known as gel filtration chromatography) separates the molecular forms of a protein based on their size. In this method, larger molecular species (e.g. aggregates, IgG dimers and oligomers) elute earlier than the desired (monomeric) IgG species as pre-peaks. Smaller molecular species (e.g. degradation products and fragments) elute later as post-peaks. SE-UPLC was performed on the antibody stability samples. Briefly, samples were diluted to 0.5 mL/min with mobile phase and 2.5 μg was injected on a Waters Acquity UPLC BEH 200 SEC, 1.7 μm, 4.6×300 mm column. Chromatographic separation occurred at ambient temperature, with a flow of 0.2 mL/min of mobile phase 10 mM phosphate, 0.4 M NaCl, pH 7.0±0.1.

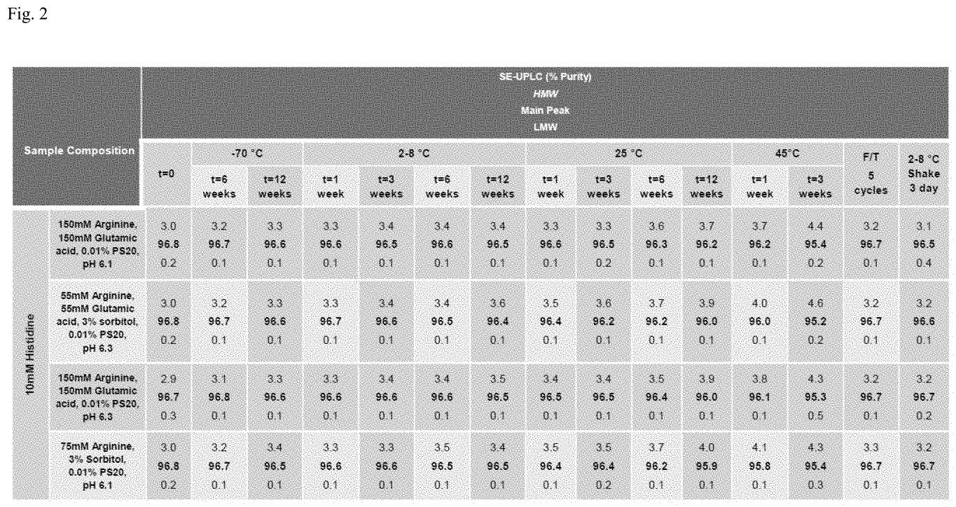

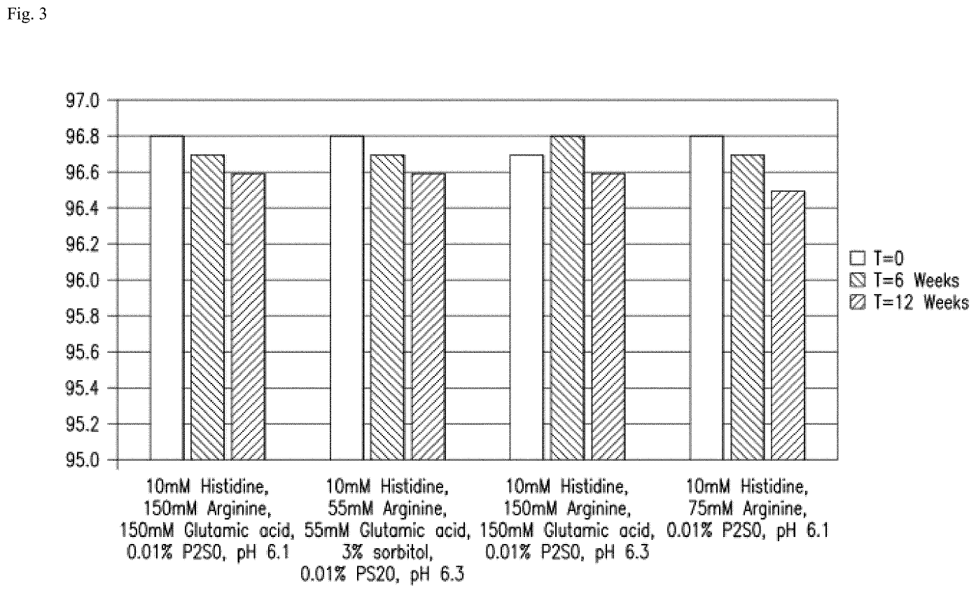

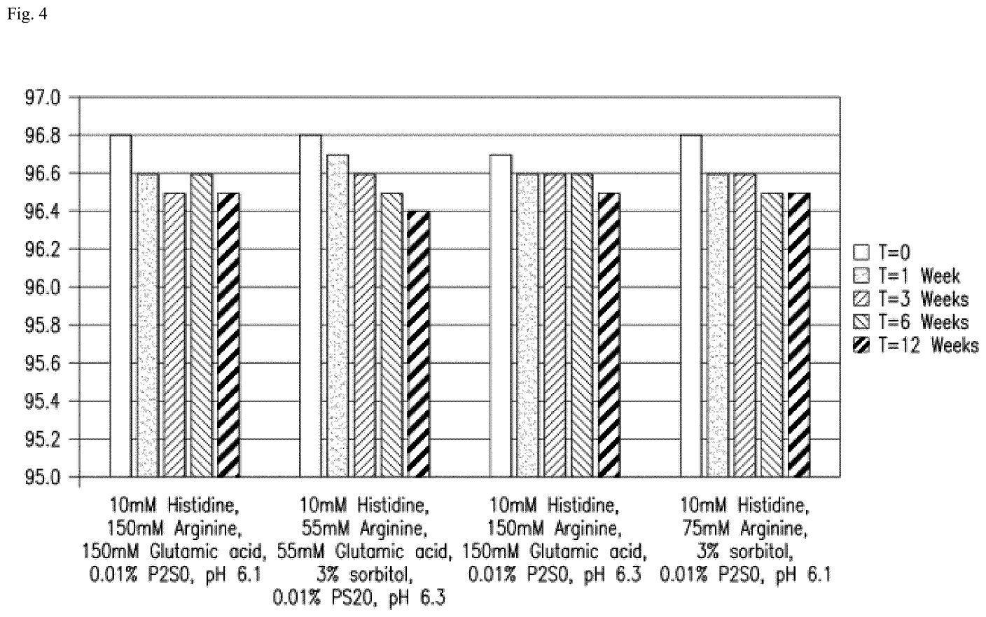

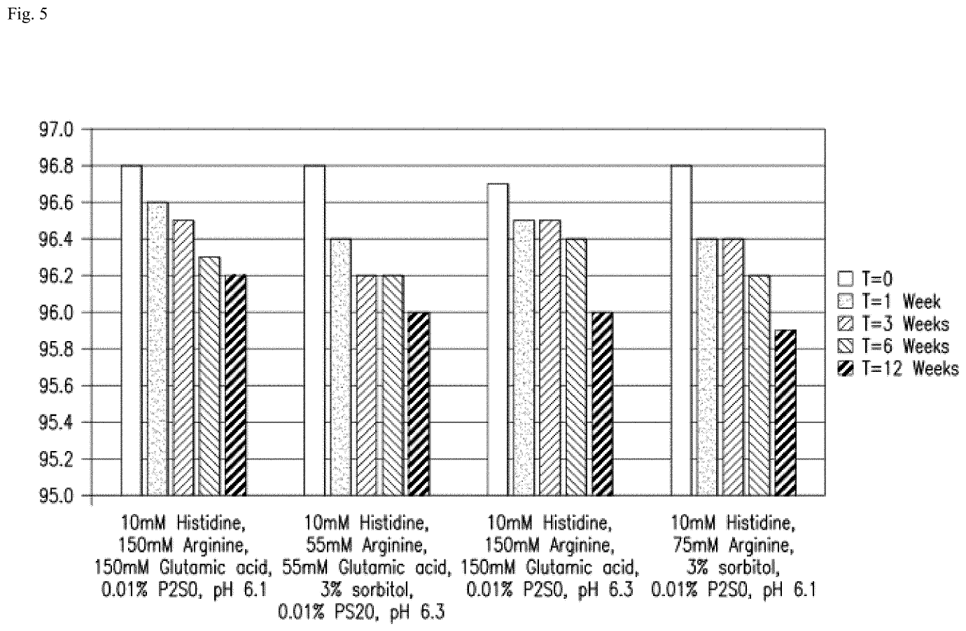

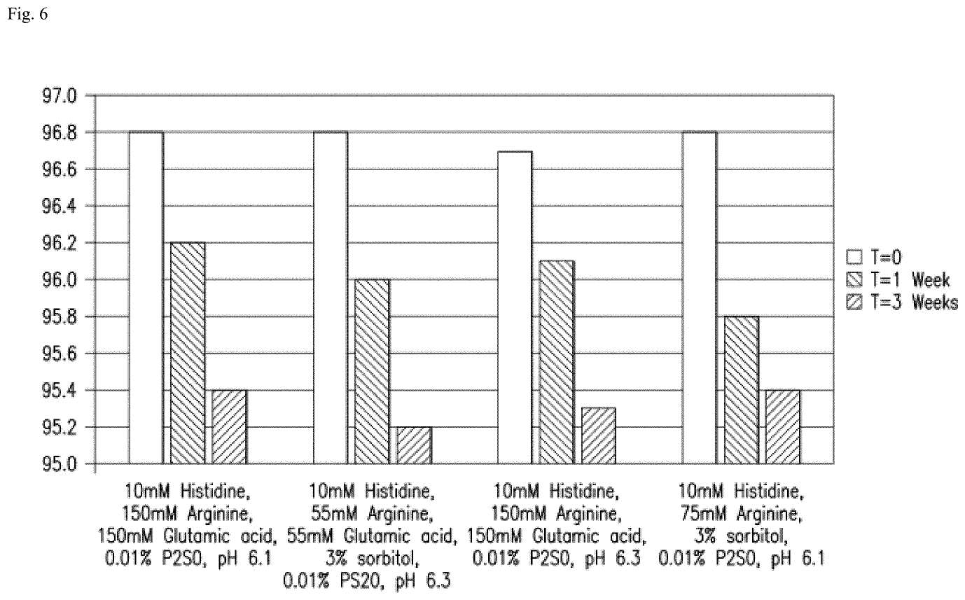

Tabular data obtained by SE-UPLC are shown in FIG. 2 and graphical representations of the relative main peaks are shown in FIGS. 3-6. Following 12 weeks incubation at 2-8° C., main peak decreased by 0.1-0.3% for all formulations. No discernable differences were observed between any of the test formulations when exposed to 5 cycles of freeze/thaw and 2-8° C. shake stress conditions as the resulting data was comparable to T=0 results and within assay variability.

Following the 12 weeks incubation at 2-8° C., each formulation displayed robust stability, with main peaks within 0.2-0.4% of the initial values. Freeze/Thaw stability as well as 2-8° C. shake stress stability was equivalent for all tested formulations. The 12 week incubation at 25° C. revealed a slightly higher purity in formulation 1 (10 mM Histidine, 150 mM Arginine, 150 mM Glutamic acid, 0.01% PS20, pH 6.1) as compared to the other formulations, but this was not the case following three weeks at 45° C. in which a drop of 1.3-1.6% in main peak area was observed.

### Example 5

Ion exchange chromatography separates molecules based on differences in their accessible surface charges. Binding is dependent on the ionic attraction between molecules of the opposite electric charge. The analysis applied herein utilized a weak cation exchange (CEX-UPLC) column. Elution is accomplished with a gradient of increasing pH. The pH gradient separations are performed using an ultrahigh performance liquid chromatography system (Thermo Vanquish) equipped with a UV detector monitoring at 280 nm (A280) and integration software (Chromeleon ver. 7.2). A Dionex ProPac WCX-10 column (4.0×250 mm) was used in this application to resolve and provide a profile of measurable populations of antibody charged species over the course of the stability study described herein. CEX-UPLC was performed on the anti-APRIL antibody stability samples acco. Samples were diluted to 1 mg/mL and 25 μg were injected on the column.

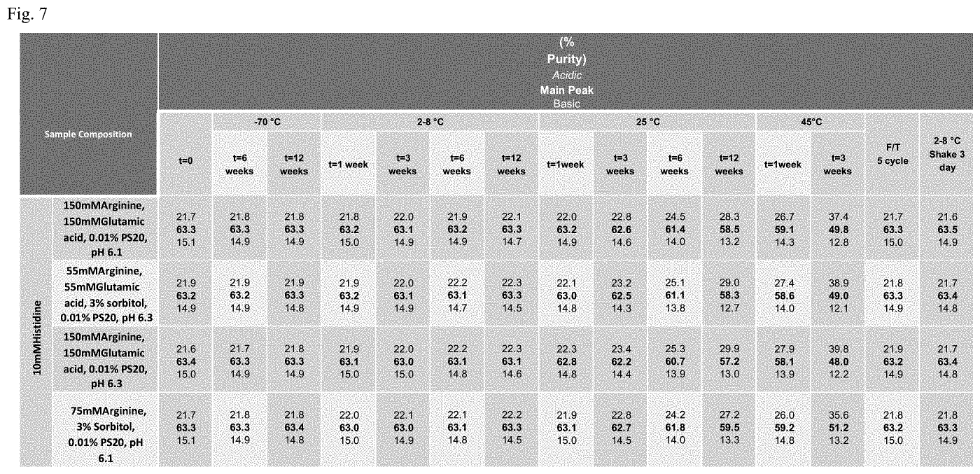

Tabular data obtained by CEX-UPLC is are shown in FIG. 7. Following 12 weeks of incubation at 2-8° C. as well as −70° C., CEX-UPLC showed main peak changed by 0.1-0.3% and no discernable difference or trend was observed within the four formulations. Following incubation at 25° C. and 45° C., main peak and basic peak levels were observed to decrease over the duration of the stability study resulting in an increase of acidic peaks for all the formulations. These differences were observed to be lower in formulations at pH 6.1 over pH 6.3. Formulation 11 (10 mM Histidine, 75 mM Arginine, 3% Sorbitol, 0.01% PS20, pH 6.1) showed the best stability by CEX-UPLC. No clear difference was observed within the four formulations following 5 cycles of freeze/thaw or following 2-8° C. shake stress.

No significant changes were observed between any of the test formulations follow 12 weeks of storage at −70° C. and 2-8° C. or upon exposure to freeze/thaw and 2-8° C. shake stress conditions as the resulting data was comparable to T=0 results and within assay variability. Following the incubation at 25° C. and 45° C., main peak and basic peak levels were observed to decrease over the duration of the stability study resulting in an increase of acidic peaks for all the formulations. These differences were observed to be lower at pH 6.1 than at pH 6.3 for VH14_1G.VL15 formulations. Formulation 11 (10 mM Histidine, 75 mM Arginine, 3% Sorbitol, 0.01% PS20, pH 6.1) showed the best stability by CEX-UPLC.

### Example 6

A qualitative review of the stability data indicates that greater VH14_1G.VL15 stability, as measured by SEC sample purity, was maintained in samples formulation 1 (10 mM Histidine, 150 mM Arginine, 150 mM Glutamic acid, 0.01% PS20, pH 6.1), which was only observable at the 25° C. and 45° C. test condition. The relationship between the rate of a reaction and its temperature is described by the Arrhenius equation.

ln k_{obs}=(−Eₐ/R)T+ln A Equation 1

where k is the rate coefficient, the pre-exponential factor, A, is a usually a constant across small temperature ranges, which includes variables like the frequency of collisions and their orientation, Ea is the activation energy, R is the universal gas constant, and T is the absolute temperature. This equation allows one to estimate reaction rates, such as degradation, at a given temperature assuming that the decomposition is thermal in nature (e.g., as opposed to light-induced) and the decomposition mechanism does not change with temperature. If these criteria are satisfied then the activation energy, Ea, and the pre-exponential factor, A, should not vary across a small temperature range, facilitating the extrapolation to other (often lower) temperatures.

Under such circumstances, experiments conducted under elevated temperature (accelerated) conditions yield valuable information about the degradation rate at lower temperatures, for which real-time degradation monitoring would be prohibitively slow and costly. Although accelerated studies provide rapid insight into stability concerns, they are not to be taken as a substitute for real-time stability studies.

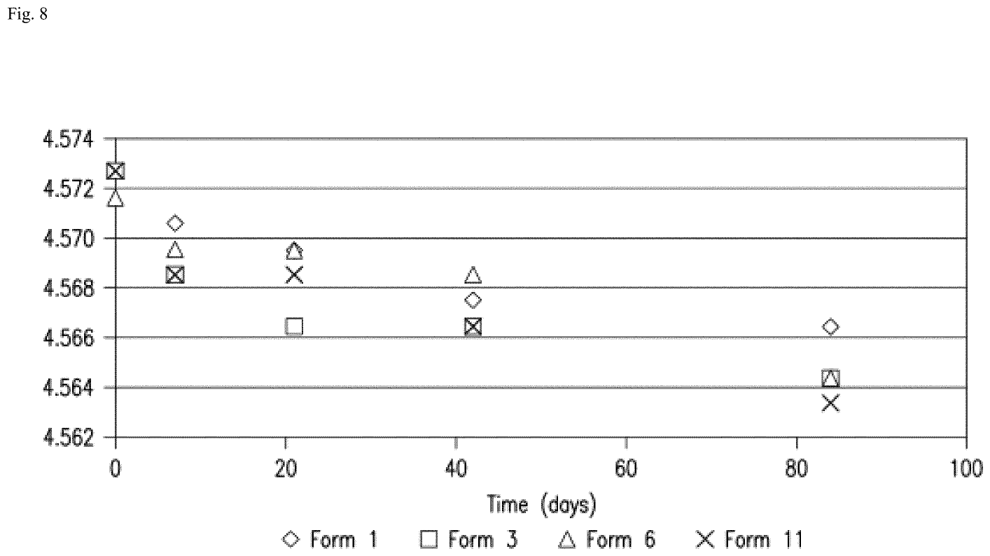

To approach data analysis in a more rigorous fashion, rate constants were determined for the loss of anti-APRIL (VH14_1G.VL15) antibody purity for each formulation and temperature condition to approximate first-order degradation kinetics. For the 2-8° C., 25° C. and 45° C. testing conditions, a plot of ln purity versus time was made using the percent antibody purity values found in the SE-UPLC analysis. The results are graphically represented in FIG. 8. From the data, the slope is correlated to a rate constant (−k_{obs}) for degradation. Results of the vehicle and temperature dependence on k_{obs} are shown in the following Table 12.

TABLE 12
Vehicle and temperature dependence on k_{obs}:

| | 45° C. | | 25° C. | | 5° C. | |
|---|---|---|---|---|---|---|
| | k_{obs} | R² | k_{obs} | R² | k_{obs} | R² |
| Form 1 | −0.0006798 | 0.98885 | −0.000068 | 0.85459 | −2.37919 × 10⁻⁵ | 0.40246 |
| Form 3 | −0.0007657 | 0.96517 | −0.000077 | 0.68408 | −4.60249 × 10⁻⁵ | 0.90271 |
| Form 6 | −0.0006805 | 0.98885 | −0.000077 | 0.93795 | −1.90437 × 10⁻⁵ | 0.77275 |
| Form 11 | −0.0006373 | 0.82541 | −0.000094 | 0.85977 | −2.85547 × 10⁻⁵ | 0.57972 |

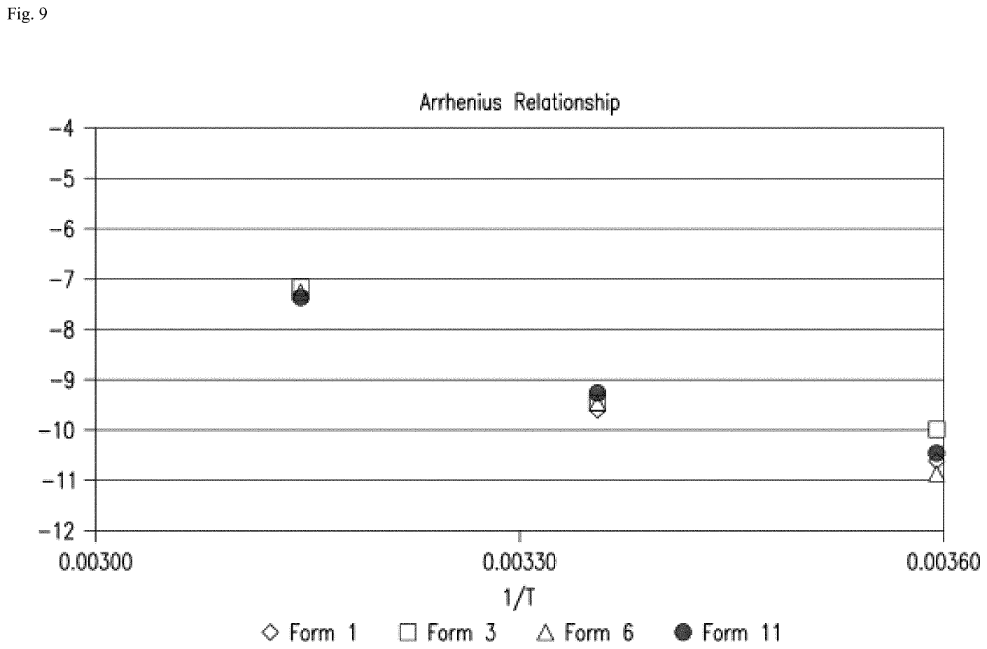

Using the data from Table 12, Arrhenius plots (ln k_{obs} versus 1/T) were generated for each of the anti-APRIL antibody formulations at three stability temperatures (FIG. 9). From this analysis, the fitted parameters of line were obtained (Table 13). A good linear fit of ln kobs versus 1/T indicates a similar decomposition mechanism operating within the temperature ranges tested. Furthermore, this implies that decomposition follows Arrhenius behavior.

TABLE 13
Fitted parameters resulting from linear regression analysis
of the ln k_{obs} vs. 1/T for each formulation:

| Formulation | Slope | Intercept | R² |
|---|---|---|---|
| Form 1 | −7343.5 | 15.52 | 0.9375 |
| Form 3 | −6122.8 | 11.72 | 0.8558 |
| Form 6 | −7861.2 | 17.24 | 0.9733 |
| Form 11 | −6823.9 | 13.92 | 0.9701 |

From the Arrhenius relationship of Equation 1, in which slope=−Ea/R and intercept=ln A, the data was then used to calculate ln kobs for each vehicle at 5° C. and 25° C. Moreover, from kobs at 5° C. and using a linear form of the rate equation for first order kinetics shown in Equation 2, where k is an observed rate constant (kobs) and Purity₀ and Purityₜ are the purity at time 0 and t, respectively, the t_{95%} was calculated which corresponds to days of incubation until VH14_1G.VL15 solution purity would approach 95% of the total sample composition (or 98.1% of the original purity). Solution stability was calculated to be ranging from 1.55 to 3.06 years at 5° C. or 0.35 to 0.47 years at 25° C. Results are provided in Table 14.

ln(Purityₜ/Purity₀)=k_{obs}t Equation 2

TABLE 14
Rates of degradation and t_{95%} for storage at 5° C. and 25° C.:

| | | | t_{95%} | | Observed % | |
|---|---|---|---|---|---|---|
| | k_{obs} | | (years) | | purity at 84 days | |
| | 5° C. | 25° C. | 5° C. | 25° C. | 2-8° C. | 25° C. |
| 1: 10 mM Histidine, 150 mM Arginine, | 1.89E−05 | 0.000111 | 2.78 | 0.47 | 96.5 | 96.2 |
| 150 mM Glutamic acid, 0.01% PS20, | | | | | | |
| pH 6.1 | | | | | | |
| 3: 10 mM Histidine, 55 mM Arginine, | 3.39E−05 | 0.000148 | 1.55 | 0.35 | 96.4 | 96.0 |
| 55 mM Glutamic acid, 3% sorbitol, | | | | | | |
| 0.01% PS20, pH 6.3 | | | | | | |
| 6: 10 mM Histidine, 150 mM Arginine, | 1.63E−05 | 0.000108 | 3.06 | 0.46 | 96.5 | 96.0 |
| 150 mM Glutamic acid, 0.01% PS20, | | | | | | |
| pH 6.3 | | | | | | |
| 11: 10 mM Histidine, 75 mM Arginine, | 2.47E−05 | 0.000128 | 2.13 | 0.41 | 96.5 | 95.9 |
| 3% Sorbitol, 0.01% PS20, pH 6.1 | | | | | | |

### Example 7

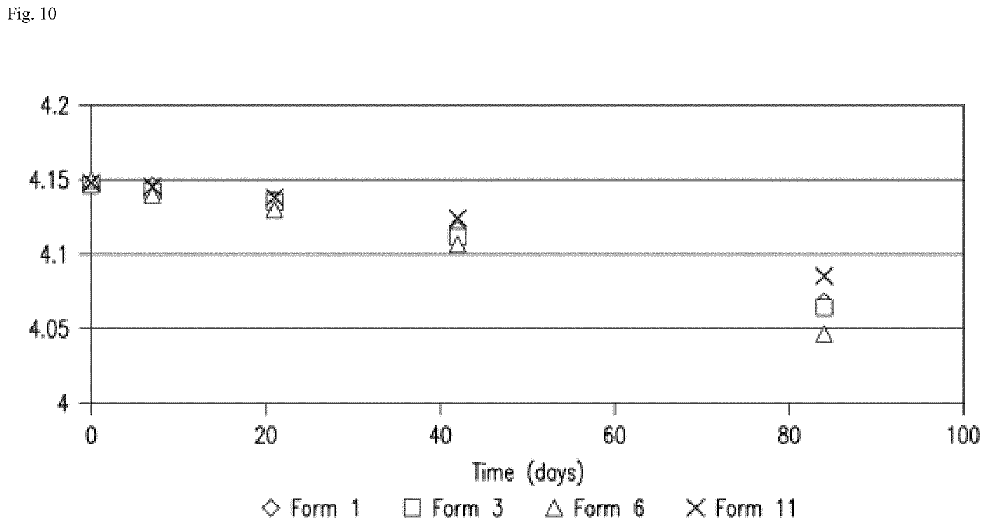

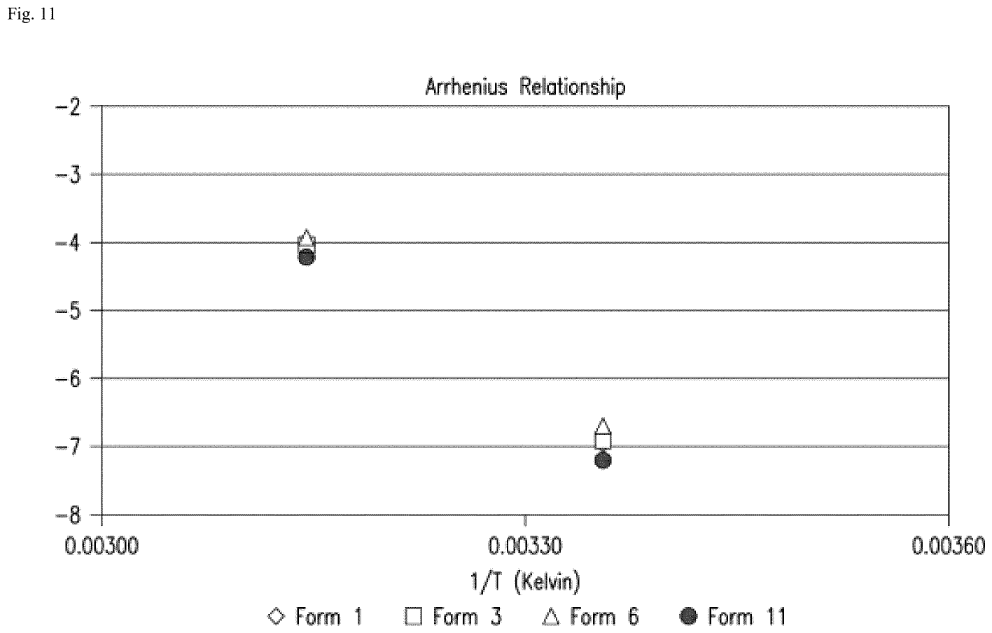

Following the same quantitative approach, the sample purity out to 95% of the original value can be estimated using the collected CEX-UPLC data. CEX-UPLC data was used to plot of ln purity versus time represented in FIG. 10. Lines generated using the data indicate pseudo first-order degradation kinetics as indicated in the following Table 15, at least for the 45° C. and 25° C. test samples, from which the slope is correlated to a rate constant (−k_{obs}) for degradation. Arrhenius plots were generated for each of the VH14_1G.VL15 formulations at 45° C. and 25° C. (FIG. 11). The 2-8° C. data was not included in the AR calculation as non-pseudo first order changes were observed.

TABLE 15
Line data from lines generated in FIG. 10.

| | 45° C. | | 25° C. | | 2-8° C. | |
|---|---|---|---|---|---|---|
| | k_{obs} | R² | k_{obs} | R² | K_{obs} | R² |
| Form 1 | −0.0171336 | 0.94256 | −0.000961 | 0.98253 | 1.1153 × 10⁻⁵ | 0.08105 |
| Form 3 | −0.0181774 | 0.94789 | −0.000983 | 0.98618 | 1.79324 × 10⁻⁵ | 0.20944 |
| Form 6 | −0.0198759 | 0.95578 | −0.001215 | 0.98971 | −2.85854 × 10⁻⁵ | 0.16211 |
| Form 11 | −0.0151533 | 0.95665 | −0.000743 | 0.98310 | −4.26581 × 10⁻⁵ | 0.30740 |

From the Arrhenius relationship, the data from Table 15 was then used to calculate ln kobs for each vehicle at 5° C. and 25° C. Moreover, from kobs and using a linear form of the rate equation for first order kinetics where k is an observed rate constant (kobs) and Purity₀ and Purityₜ, the t_{95%} was calculated which corresponds to years of incubation until VH14_1G.VL15 solution purity would approach 95% of the original sample purity (see Tables 16 and 17). Solution stability was calculated to be ranging from 2.83 to 5.95 years at 5° C. and from 0.12-0.19 years at 25° C.

TABLE 16
Fitted parameters resulting from linear regression analysis
of the ln k_{obs} vs. 1/T for each formulation:

| Formulation | Slope | Intercept |
|---|---|---|
| Form 1 | −13661.8 | 38.87 |
| Form 3 | −13838.4 | 39.49 |
| Form 6 | −13255.1 | 37.74 |
| Form 11 | −14300.4 | 40.76 |

TABLE 17
Rates of degradation and t_{95%} for storage at 5° C. and 25° C.

| | | | t_{95%} | | Obs. % main | |
|---|---|---|---|---|---|---|
| | k_{obs} | | (years) | | peak at 84 days | |
| | 5° C. | 25° C. | 5° C. | 25° C. | 2-8° C. | 25° C. |
| Form1: 10 mM Histidine, 150 mM Arginine, | 3.56E−05 | 0.000961 | 3.94 | 0.15 | 63.3 | 58.5 |
| 150 mM Glutamic acid, 0.01% PS20, pH 6.1 | | | | | | |
| Form 3: 10 mM Histidine, 55 mM Arginine, | 3.49E−05 | 0.000983 | 4.03 | 0.14 | 63.3 | 58.3 |
| 55 mM Glutamic acid, 3% sorbitol, 0.01% | | | | | | |
| PS20, pH 6.3 | | | | | | |
| Form 6: 10 mM Histidine, 150 mM Arginine, | 4.97E−05 | 0.001215 | 2.83 | 0.12 | 63.1 | 57.2 |
| 150 mM Glutamic acid, 0.01% PS20, pH 6.3 | | | | | | |
| Form 11: 10 mM Histidine, 75 mM Arginine, | 2.36E−05 | 0.000743 | 5.95 | 0.19 | 63.3 | 59.5 |
| 3% Sorbitol, 0.01% PS20, pH 6.1 | | | | | | |

### Example 8

Dynamic light scattering (DLS) is used to determine a molecule's hydrodynamic radius. It measures translational diffusion of the molecules in solution by capturing the scattered light intensity fluctuation due to the molecules' Brownian motion. If the molecule is assumed to be a uniform sphere, the diffusion coefficient can be translated into the molecule's hydrodynamic radius by the Stokes-Einstein relationship. DLS analysis was performed on the VH14_1G.VL15 stability samples for each time point. Briefly, a buffer blank was initially performed to measure the scattering from each formulation; this value is then subtracted from the test sample reading to minimize buffer effects. A volume of 10 μL of each undiluted, unfiltered test sample were loaded in a clean 1 μL NanoStar quartz cuvette and analyzed over a measurement time period of 50 seconds acquisitions for 5 seconds each at 25° C. In this study, the DLS intensity autocorrelation data for the diffusion coefficient determination was analyzed by the regularization method, which provides estimates of the radii and relative abundance of all species present in solution. As described in Wyatt Technology DYNAMIC 7.1.9, acceptable DLS data should have a smooth and continuous auto-correlation function exponentially decaying from a maximum intensity correlation value of 2 to a value of 1. All test samples displayed acceptable decay.

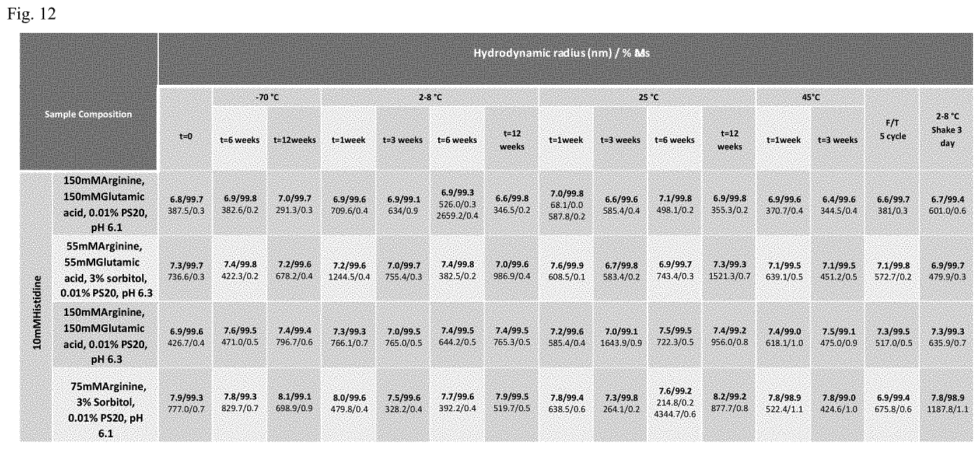

Hydrodynamic radii of the observable particle species are summarized in FIG. 12. Data in the summary table are the values obtained for the first measurement which contains ten acquisitions. Detailed results of the main population for each measurement are shown in the following Tables 18-21.

TABLE 18
Radius, polydispersity, and abundance of the main population
of species in formulation 1 samples:

| | Radius | % | % | % | % |
|---|---|---|---|---|---|
| | (nm) | Pd | Mass | Intensity | Number |
| Formulation 1, t = 0 | 6.8 | 7.8 | 99.7 | 92.5 | 100.0 |
| Formulation 1, t = 0 | 7.1 | 19.3 | 99.6 | 93.8 | 100.0 |
| Formulation 1, t = 3 days, | 6.7 | 2.9 | 99.4 | 92.1 | 100.0 |
| Shake | | | | | |
| Formulation 1, t = 3 days, | 7.1 | 22.5 | 99.6 | 91.9 | 100.0 |
| Shake | | | | | |
| Formulation 1, t = 1 week, | 6.6 | 5.9 | 99.7 | 91.7 | 100.0 |
| F/T | | | | | |
| Formulation 1, t = 1 week, | 6.8 | 17.7 | 99.5 | 92.5 | 100.0 |
| F/T | | | | | |
| Formulation 1, t = 1 week, | 6.9 | 20.1 | 99.6 | 94.4 | 100.0 |
| 2-8° C. | | | | | |
| Formulation 1, t = 1 week, | 6.8 | 17.8 | 99.8 | 93.0 | 100.0 |
| 2-8° C. | | | | | |
| Formulation 1, t = 1 week, | 7.0 | 24.5 | 99.8 | 94.7 | 100.0 |
| 25° C. | | | | | |
| Formulation 1, t = 1 week, | 6.8 | 11.7 | 99.8 | 93.5 | 100.0 |
| 25° C. | | | | | |
| Formulation 1, t = 1 week, | 6.9 | 29.7 | 99.6 | 86.8 | 100.0 |
| 45° C. | | | | | |
| Formulation 1, t = 1 week, | 6.5 | 16.6 | 99.6 | 87.3 | 100.0 |
| 45° C. | | | | | |
| Formulation 1, t = 3 week, | 6.9 | 7.6 | 99.1 | 89.0 | 100.0 |
| 2-8° C. | | | | | |
| Formulation 1, t = 3 week, | 7.0 | 16.2 | 99.6 | 93.8 | 100.0 |
| 2-8° C. | | | | | |
| Formulation 1, t = 3 week, | 6.6 | 7.3 | 99.6 | 92.8 | 100.0 |
| 25° C. | | | | | |
| Formulation 1, t = 3 week, | 6.7 | 17.9 | 99.9 | 95.1 | 100.0 |
| 25° C. | | | | | |
| Formulation 1, t = 3 week, | 6.4 | 9.5 | 99.6 | 85.7 | 100.0 |
| 45° C. | | | | | |
| Formulation 1, t = 3 week, | 6.4 | 18.0 | 99.4 | 84.2 | 100.0 |
| 45° C. | | | | | |
| Formulation 1, t = 6 week, | 6.9 | 17.4 | 99.8 | 93.0 | 100.0 |
| −70° C. | | | | | |
| Formulation 1, t = 6 week, | 7.4 | 22.8 | 99.5 | 98.3 | 100.0 |
| −70° C. | | | | | |
| Formulation 1, t = 6 week, | 6.9 | 6.3 | 98.8 | 92.2 | 100.0 |
| 2-8° C. | | | | | |
| Formulation 1, t = 6 week, | 6.9 | 7.2 | 99.3 | 93.2 | 100.0 |
| 2-8° C. | | | | | |
| Formulation 1, t = 6 week, | 7.1 | 9.9 | 99.8 | 95.6 | 100.0 |
| 25° C. | | | | | |
| Formulation 1, t = 6 week, | 7.3 | 16.4 | 99.8 | 95.3 | 100.0 |
| 25° C. | | | | | |
| Formulation 1, t = 12 week, | 7.0 | 22.8 | 99.7 | 91.1 | 100.0 |
| −70° C. | | | | | |
| Formulation 1, t = 12 week, | 7.2 | 16.9 | 99.7 | 93.2 | 100.0 |
| −70° C. | | | | | |
| Formulation 1, t = 12 week, | 6.6 | 8.8 | 99.8 | 94.2 | 100.0 |
| 2-8° C. | | | | | |
| Formulation 1, t = 12 week, | 6.8 | 17.6 | 99.6 | 92.4 | 100.0 |
| 2-8° C. | | | | | |
| Formulation 1, t = 12 week, | 6.9 | 6.6 | 99.8 | 94.9 | 100.0 |
| 25° C. | | | | | |
| Formulation 1, t = 12 week, | 7.0 | 10.4 | 99.6 | 94.8 | 100.0 |
| 25° C. | | | | | |

TABLE 19
Radius, polydispersity, and abundance of the main population
of species in formulation 3 samples:

| | Radius | % | % | % | % |
|---|---|---|---|---|---|
| | (nm) | Pd | Mass | Intensity | Number |
| Formulation 3, t = 0 | 7.3 | 22.5 | 99.7 | 96.2 | 100.0 |
| Formulation 3, t = 0 | 7.4 | 25.2 | 99.7 | 96.5 | 100.0 |
| Formulation 3, t = 3 days, | 6.9 | 17.2 | 99.7 | 93.2 | 100.0 |
| Shake | | | | | |
| Formulation 3, t = 3 days, | 6.9 | 18.2 | 99.7 | 93.8 | 100.0 |
| Shake | | | | | |
| Formulation 3, t = 1 week, | 6.2 | 11.7 | 99.6 | 87.1 | 100.0 |
| F/T | | | | | |
| Formulation 3, t = 1 week, | 7.1 | 32.3 | 99.8 | 95.3 | 100.0 |
| F/T | | | | | |
| Formulation 3, t = 1 week, | 7.2 | 16.8 | 99.6 | 97.3 | 100.0 |
| 2-8° C. | | | | | |
| Formulation 3, t = 1 week, | 7.2 | 19.5 | 99.6 | 95.7 | 100.0 |
| 2-8° C. | | | | | |
| Formulation 3, t = 1 week, | 7.6 | 29.1 | 99.9 | 98.1 | 100.0 |
| 25° C. | | | | | |
| Formulation 3, t = 1 week, | 7.1 | 19.6 | 99.5 | 97.3 | 100.0 |
| 25° C. | | | | | |
| Formulation 3, t = 1 week, | 7.1 | 16.8 | 99.5 | 93.2 | 100.0 |
| 45° C. | | | | | |
| Formulation 3, t = 1 week, | 7.2 | 19.6 | 99.6 | 94.0 | 100.0 |
| 45° C. | | | | | |
| Formulation 3, t = 3 week, | 7.0 | 12.3 | 99.7 | 96.1 | 100.0 |
| 2-8° C. | | | | | |
| Formulation 3, t = 3 week, | 6.8 | 5.0 | 99.8 | 95.1 | 100.0 |
| 2-8° C. | | | | | |
| Formulation 3, t = 3 week, | 6.7 | 7.4 | 99.8 | 96.7 | 100.0 |
| 25° C. | | | | | |
| Formulation 3, t = 3 week, | 7.0 | 19.9 | 99.8 | 96.6 | 100.0 |
| 25° C. | | | | | |
| Formulation 3, t = 3 week, | 7.1 | 15.5 | 99.5 | 89.3 | 100.0 |
| 45° C. | | | | | |
| Formulation 3, t = 3 week, | 7.0 | 6.1 | 99.5 | 88.9 | 100.0 |
| 45° C. | | | | | |
| Formulation 3, t = 6 week, | 6.9 | 4.5 | 99.8 | 92.8 | 100.0 |
| −70° C. | | | | | |
| Formulation 3, t = 6 week, | 7.4 | 17.9 | 99.8 | 95.2 | 100.0 |
| −70° C. | | | | | |
| Formulation 3, t = 6 week, | 7.4 | 13.6 | 99.8 | 95.8 | 100.0 |
| 2-8° C. | | | | | |
| Formulation 3, t = 6 week, | 7.2 | 10.6 | 99.6 | 95.5 | 100.0 |
| 2-8° C. | | | | | |
| Formulation 3, t = 6 week, | 6.9 | 2.9 | 99.7 | 95.8 | 100.0 |
| 25° C. | | | | | |
| Formulation 3, t = 6 week, | 7.3 | 16.8 | 99.1 | 97.2 | 100.0 |
| 25° C. | | | | | |
| Formulation 3, t = 12 week, | 7.2 | 15.2 | 99.6 | 95.3 | 100.0 |
| −70° C. | | | | | |
| Formulation 3, t = 12 week, | 7.2 | 20.8 | 99.8 | 95.4 | 100.0 |
| −70° C. | | | | | |
| Formulation 3, t = 12 week, | 7.0 | 7.6 | 99.6 | 96.4 | 100.0 |
| 2-8° C. | | | | | |
| Formulation 3, t = 12 week, | 7.0 | 9.2 | 99.6 | 96.7 | 100.0 |
| 2-8° C. | | | | | |
| Formulation 3, t = 12 week, | 7.3 | 10.6 | 99.3 | 96.6 | 100.0 |
| 25° C. | | | | | |
| Formulation 3, t = 12 week, | 7.2 | 9.1 | 99.2 | 96.8 | 100.0 |
| 25° C. | | | | | |

TABLE 20
Radius, polydispersity, and abundance of the main population
of species in formulation 6 samples:

| | Radius | % | % | % | % |
|---|---|---|---|---|---|
| | (nm) | Pd | Mass | Intensity | Number |
| Formulation 6, t = 0 | 6.9 | 17.7 | 99.6 | 90.5 | 100.0 |
| Formulation 6, t = 0 | 6.6 | 16.3 | 99.7 | 87.4 | 100.0 |
| Formulation 6, t = 3 days, | 7.3 | 9.4 | 99.3 | 92.5 | 100.0 |
| Shake | | | | | |
| Formulation 6, t = 3 days, | 7.4 | 11.7 | 99.3 | 93.4 | 100.0 |
| Shake | | | | | |
| Formulation 6, t = 1 week, | 7.3 | 19.7 | 99.5 | 91.5 | 100.0 |
| F/T | | | | | |
| Formulation 6, t = 1 week, | 7.3 | 17.5 | 99.4 | 92.3 | 100.0 |
| F/T | | | | | |
| Formulation 6, t = 1 week, | 7.3 | 10.3 | 99.3 | 93.2 | 100.0 |
| 2-8° C. | | | | | |
| Formulation 6, t = 1 week, | 7.4 | 10.6 | 99.3 | 93.5 | 100.0 |
| 2-8° C. | | | | | |
| Formulation 6, t = 1 week, | 7.2 | 12.2 | 99.6 | 94.5 | 100.0 |
| 25° C. | | | | | |
| Formulation 6, t = 1 week, | 7.0 | 7.6 | 99.7 | 93.0 | 100.0 |
| 25° C. | | | | | |
| Formulation 6, t = 1 week, | 7.4 | 21.1 | 99.0 | 87.2 | 100.0 |
| 45° C. | | | | | |
| Formulation 6, t = 1 week, | 7.4 | 22.0 | 99.1 | 87.5 | 100.0 |
| 45° C. | | | | | |
| Formulation 6, t = 3 week, | 7.0 | 10.0 | 99.5 | 94.0 | 100.0 |
| 2-8° C. | | | | | |
| Formulation 6, t = 3 week, | 6.9 | 8.6 | 99.5 | 93.3 | 100.0 |
| 2-8° C. | | | | | |
| Formulation 6, t = 3 week, | 7.0 | 7.2 | 99.1 | 95.3 | 100.0 |
| 25° C. | | | | | |
| Formulation 6, t = 3 week, | 7.1 | 11.8 | 99.6 | 95.8 | 100.0 |
| 25° C. | | | | | |
| Formulation 6, t = 3 week, | 7.3 | 13.2 | 98.9 | 83.4 | 100.0 |
| 45° C. | | | | | |
| Formulation 6, t = 3 week, | 7.5 | 23.6 | 99.1 | 83.8 | 100.0 |
| 45° C. | | | | | |
| Formulation 6, t = 6 week, | 7.6 | 15.3 | 99.5 | 91.9 | 100.0 |
| -70° C. | | | | | |
| Formulation 6, t = 6 week, | 7.6 | 16.7 | 99.3 | 91.7 | 100.0 |
| -70° C. | | | | | |
| Formulation 6, t = 6 week, | 7.4 | 15.7 | 99.5 | 94.1 | 100.0 |
| 2-8° C. | | | | | |
| Formulation 6, t = 6 week, | 7.4 | 10.4 | 99.4 | 94.0 | 100.0 |
| 2-8° C. | | | | | |
| Formulation 6, t = 6 week, | 7.5 | 15.1 | 99.5 | 95.3 | 100.0 |
| 25° C. | | | | | |
| Formulation 6, t = 6 week, | 7.7 | 23.4 | 99.5 | 95.9 | 100.0 |
| 25° C. | | | | | |
| Formulation 6, t = 12 week, | 7.4 | 13.5 | 99.4 | 94.0 | 100.0 |
| −70° C. | | | | | |
| Formulation 6, t = 12 week, | 7.4 | 15.8 | 99.4 | 93.2 | 100.0 |
| −70° C. | | | | | |
| Formulation 6, t = 12 week, | 7.4 | 13.5 | 99.5 | 95.3 | 100.0 |
| 2-8° C. | | | | | |
| Formulation 6, t = 12 week, | 7.4 | 13.8 | 99.6 | 95.0 | 100.0 |
| 2-8° C. | | | | | |
| Formulation 6, t = 12 week, | 7.4 | 10.4 | 99.2 | 94.2 | 100.0 |
| 25° C. | | | | | |
| Formulation 6, t = 12 week, | 7.3 | 10.9 | 99.4 | 94.4 | 100.0 |
| 25° C. | | | | | |

TABLE 21
Radius, polydispersity, and abundance of the main population
of species in formulation 11 samples:

| | Radius | % | % | % | % |
|---|---|---|---|---|---|
| | (nm) | Pd | Mass | Intensity | Number |
| Formulation 11, t = 0 | 7.9 | 13.9 | 99.3 | 94.2 | 100.0 |
| Formulation 11, t = 0 | 7.7 | 11.8 | 99.2 | 93.6 | 100.0 |
| Formulation 11, t = 3 days, | 7.8 | 12.9 | 98.9 | 94.0 | 100.0 |
| Shake | | | | | |
| Formulation 11, t = 3 days, | 7.9 | 16.7 | 99.1 | 92.1 | 100.0 |
| Shake | | | | | |
| Formulation 11, t = 1 week, | 6.9 | 17.4 | 99.4 | 92.0 | 100.0 |
| F/T | | | | | |
| Formulation 11, t = 1 week, | 6.6 | 11.9 | 99.8 | 86.7 | 100.0 |
| F/T | | | | | |
| Formulation 11, t = 1 week, | 8.0 | 23.0 | 99.6 | 93.4 | 100.0 |
| 2-8° C. | | | | | |
| Formulation 11, t = 1 week, | 7.7 | 13.1 | 99.3 | 93.6 | 100.0 |
| 2-8° C. | | | | | |
| Formulation 11, t = 1 week, | 7.8 | 14.7 | 99.4 | 93.9 | 100.0 |
| 25° C. | | | | | |
| Formulation 11, t = 1 week, | 7.3 | 10.5 | 98.0 | 91.8 | 100.0 |
| 25° C. | | | | | |
| Formulation 11, t = 1 week, | 7.8 | 12.2 | 98.9 | 87.1 | 100.0 |
| 45° C. | | | | | |
| Formulation 11, t = 1 week, | 7.9 | 19.8 | 98.9 | 87.0 | 100.0 |
| 45° C. | | | | | |
| Formulation 11, t = 3 week, | 7.5 | 16.6 | 99.6 | 91.7 | 100.0 |
| 2-8° C. | | | | | |
| Formulation 11, t = 3 week, | 7.8 | 24.1 | 99.6 | 94.1 | 100.0 |
| 2-8° C. | | | | | |
| Formulation 11, t = 3 week, | 7.3 | 10.6 | 99.8 | 94.4 | 100.0 |
| 25° C. | | | | | |
| Formulation 11, t = 3 week, | 7.3 | 9.6 | 99.4 | 93.9 | 100.0 |
| 25° C. | | | | | |
| Formulation 11, t = 3 week, | 7.8 | 36.0 | 99.0 | 78.5 | 100.0 |
| 45° C. | | | | | |
| Formulation 11, t = 3 week, | 7.3 | 21.6 | 98.7 | 78.8 | 100.0 |
| 45° C. | | | | | |
| Formulation 11, t = 6 week, | 7.8 | 22.8 | 99.3 | 93.9 | 100.0 |
| -70° C. | | | | | |
| Formulation 11, t = 6 week, | 7.8 | 23.9 | 99.4 | 93.4 | 100.0 |
| -70° C. | | | | | |
| Formulation 11, t = 6 week, | 7.7 | 11.9 | 99.6 | 92.9 | 100.0 |
| 2-8° C. | | | | | |
| Formulation 11, t = 6 week, | 8.3 | 12.0 | 99.5 | 94.8 | 100.0 |
| 2-8° C. | | | | | |
| Formulation 11, t = 6 week, | 7.6 | 11.6 | 99.2 | 90.9 | 100.0 |
| 25° C. | | | | | |
| Formulation 11, t = 6 week, | 7.6 | 12.0 | 98.9 | 91.4 | 100.0 |
| 25° C. | | | | | |
| Formulation 11, t = 12 week, | 8.1 | 13.4 | 99.1 | 93.2 | 100.0 |
| −70° C. | | | | | |
| Formulation 11, t = 12 week, | 8.1 | 17.1 | 99.2 | 93.1 | 100.0 |
| −70° C. | | | | | |
| Formulation 11, t = 12 week, | 7.9 | 12.2 | 99.5 | 94.5 | 100.0 |
| 2-8° C. | | | | | |
| Formulation 11, t = 12 week, | 8.2 | 16.3 | 99.3 | 93.8 | 100.0 |
| 2-8° C. | | | | | |
| Formulation 11, t = 12 week, | 8.2 | 15.8 | 99.2 | 95.0 | 100.0 |
| 25° C. | | | | | |
| Formulation 11, t = 12 week, | 8.2 | 11.2 | 98.9 | 95.0 | 100.0 |
| 25° C. | | | | | |

At the start of the study, a bimodal distribution was observed for samples at T=0, with an additional population showing hydrodynamic radius in the >10² nm range. The polydispersity, presented in Table 18 to Table 21 ranged from 7.8% to 25.2% for samples at T=0. Upon study initiation, the main population represented the major species with % Mass≥99.3%. Overall, the particle size and relative abundance of the main population remained comparable throughout the study, regardless of storage condition. DLS measurements revealed hydrodynamic particle size ranges from 6.8 to 7.9 nm for depending upon the formulation composition. A bimodal distribution was observed for samples at T=0, with an additional population showing hydrodynamic radius in the >10² nm range. Upon study initiation, the main population represented the major species with % Mass≥99.3%. Overall, the particle size and relative abundance of the main population remained comparable throughout the study, regardless of storage condition or formulation.

### Example 9

A total of five anti-APRIL (VH14_1G.VL15) antibody formulations with four at 20 mg/mL and one at 50 mg/mL were prepared containing histidine buffer over the range of pH 6.0 to 6.5 (Table 22). A volume of 1.2 mL of test sample was aseptically placed in 2 mL glass vials which were subsequently stoppered. At each requisite time point, samples were pulled from the condition, and visual appearance was performed in the original vials. A330 and pH were analyzed on the unfiltered samples while A280, SEC and CEX were performed using 0.2 μm filtered samples to remove particles which might interfere with analysis. For HIAC analysis, a volume of 0.75 mL of the stability sample was diluted 1:1 with the corresponding buffer to provide sufficient volume for analysis. Osmolality, and viscosity were performed only at T=0 time point. Any remaining sample volume was retained at −70° C. for backup testing.

TABLE 22
List of formulations.

| | | | | | Glutamic | Sorbitol | |
|---|---|---|---|---|---|---|---|
| Formulation | | Conc. | 10 mM | Arginine | Acid | % | 0.01% (w/v) |
| ID | pH | (mg/mL) | Histidine | (mM) | (mM) | (w/w) | PS20 |
| #11 | 6.0 | 20 | + | 110 | | 1.5 | + |
| #12 | 6.3 | 20 | + | 75 | | 3 | + |
| #13 | 6.5 | 50 | + | 75 | | 3 | + |
| #14 | 6.5 | 20 | + | 75 | | 3 | + |
| #15 | 6.5 | 20 | + | 55 | 55 | 3 | + |

Test sample pH, concentration, viscosity and osmolality following preparation are recorded. Viscosity was 1.2 cP for all the four formulations at 20 mg/mL. The 50 mg/mL formulation has a slightly higher viscosity of 1.5 cP. Osmolality values ranged from 293-377 mOsm. Results are provided in the following Table 23.

TABLE 23
pH, Osmolality and Viscosity measurements

| Form # | | | Conc. | Osmolality | Viscosity |
|---|---|---|---|---|---|
| ID | pH | Meas. pH | (mg/mL) | (mOsm/kg) | (mPa · s) |
| #11 | 6.0 | 6.1 | 20.2 | 296 | 1.2 |
| #12 | 6.3 | 6.4 | 20.2 | 333 | 1.2 |
| #13 | 6.5 | 6.6 | 47.8 | 337 | 1.5 |
| #14 | 6.5 | 6.6 | 20.1 | 328 | 1.2 |
| #15 | 6.5 | 6.5 | 20.4 | 293 | 1.2 |

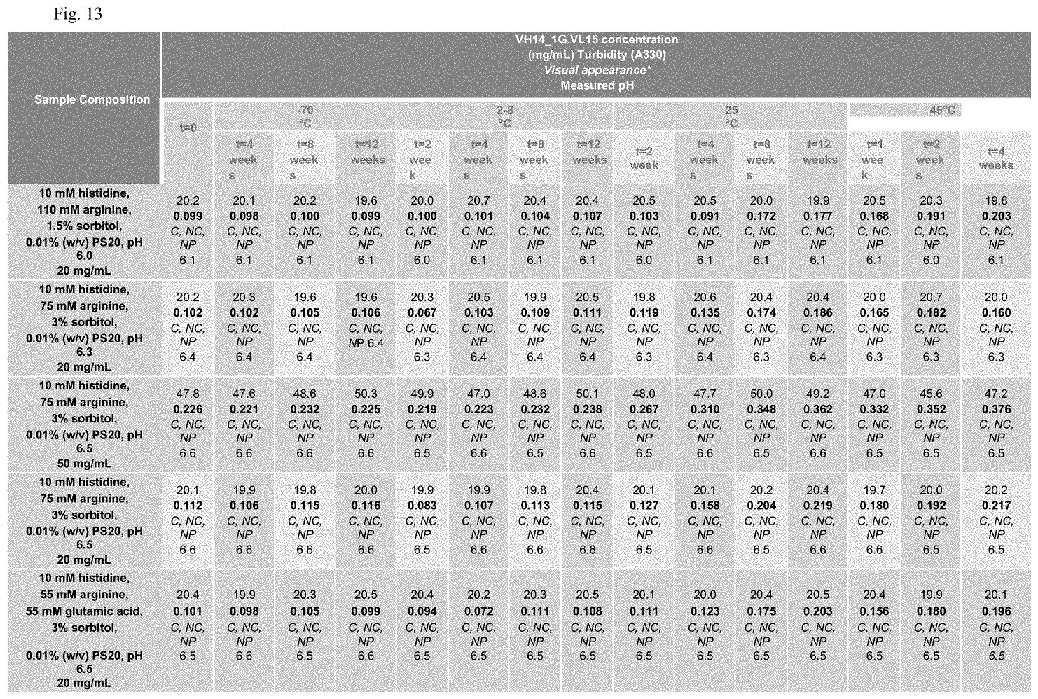

Following stress incubation, the sample visual appearance, protein concentration, turbidity, and pH were noted as in FIG. 13. No difference was observable in regards to protein concentration and pH following the different stress conditions. Visual appearance remained clear, colorless and with no visible particulates for all the formulations following the different stress conditions. Overall, turbidity values following 25° C. and 45° C. incubation indicted the greatest stability of the formulation with arginine and sorbitol at pH 6.3. No discernable analytical changes in any of the formulations following 5×F/T or 3-days shaking at 2-8° C. (Table 24).

TABLE 24
Composition, turbidity, visual appearance and pH tabular
results following freeze/thaw and shaking stress:

| | VH14_1G.VL15 | | |
|---|---|---|---|
| | concentration (mg/mL) | | |
| | Turbidity (A330) | | |
| | Visual appearance* | | |
| | Measured pH | | |
| Sample Composition | t = 0 | F/T, 5x | Shake 3 day |
| 10 mM histidine, 110 mM | 20.2 | 20.0 | 20.5 |
| arginine, 1.5% sorbitol, | 0.099 | 0.099 | 0.099 |
| 0.01% (w/v) PS20, pH 6.0, | C, NC, NP | C, NC, NP | C, NC, NP |
| 20 mg/mL | 6.1 | 6.1 | 6.1 |
| 10 mM histidine, 75 mM | 20.2 | 20.1 | 20.2 |
| arginine, 3% sorbitol, | 0.102 | 0.103 | 0.100 |
| 0.01% (w/v) PS20, pH 6.3, | C, NC, NP | C, NC, NP | C, NC, NP |
| 20 mg/mL | 6.4 | 6.4 | 6.4 |
| 10 mM histidine, 75 mM | 47.8 | 46.6 | 51.4 |
| arginine, 3% sorbitol, | 0.226 | 0.220 | 0.225 |
| 0.01% (w/v) PS20, pH 6.5 | C, NC, NP | C, NC, NP | C, NC, NP |
| 50 mg/mL | 6.6 | 6.6 | 6.6 |
| 10 mM histidine, 75 mM | 20.1 | 20.2 | 20.4 |
| arginine, 3% sorbitol, | 0.112 | 0.069 | 0.107 |
| 0.01% (w/v) PS20, pH 6.5 | C, NC, NP | C, NC, NP | C, NC, NP |
| 20 mg/mL | 6.6 | 6.6 | 6.6 |
| 10 mM histidine, 55 mM | 20.4 | 20.0 | 21.0 |
| arginine, 55 mM glutamic | 0.101 | 0.066 | 0.097 |
| acid, 3% sorbitol, | C, NC, NP | C, NC, NP | C, NC, NP |
| 0.01% (w/v) PS20, pH 6.5 | 6.5 | 6.6 | 6.6 |
| 20 mg/mL | | | |

*C = clear; O = opalescent; NC = no color; NP = no particulates

### Example 10

This procedure is applicable to all liquid samples that require particle sizing and counting tests to be performed in the Analytical and Formulation Development (AFD) laboratories using the HIAC 9703 Liquid Particle Counting System. The HIAC consists of a sampler, particle counter and Royco sensor (HRLD 400 Sensor). The Royco sensor is capable of sizing and counting particles between 2 μm to 100 μm. The instrument can count particles≤10,000 counts/mL. This method has not been validated and is for development purposes only.

Briefly, samples and controls are prepared in a biologically safe cabinet to prevent addition of particles from the environment. A volume of 0.75 mL of the stability sample was diluted 1:1 with the corresponding buffer to provide sufficient volume for analysis. Controls consist of purified water to be used as system suitability samples (used to ensure no particles were introduced during preparation) and placebo samples that are essentially buffers placed at condition.

Samples and controls are degassed for two hours and then analyzed using the HIAC system. The HIAC method consists of six consecutive runs of 0.2 mL volume from each sample. The first three runs are used to equilibrate the sensor and therefore are disregarded and the average particle counts from the final three (3) runs are averaged to give particle counts in counts/mL.

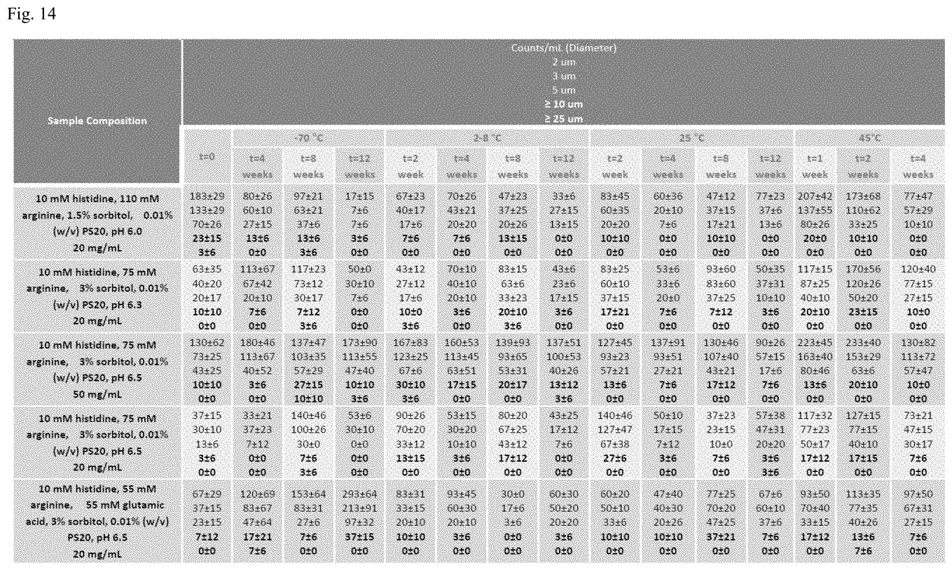

Particle sizing and counts measured by HIAC are reported in FIG. 14. No significant changes were observed between any of the test anti-APRIL (VH14_1G.VL15) antibody formulations as the resulting data was comparable to t=0 results and within assay variability; thus they were all equally stable. No significant changes were observed between any of the test formulations when exposed to freeze/thaw and 2-8° C. shake stress conditions as the resulting data was comparable to t=0 results and within assay variability (Table 25).

TABLE 25
HIAC: Tabular results of formulations following freeze/thaw and
shaking stress.

| | Counts/mL | | |
|---|---|---|---|
| | (Diameter) 2 μm | | |
| | 3 μm | | |
| | 5 μm | | |
| | ≥10 μm | | |
| | ≥25 μm | | |
| Sample Composition | t = 0 | F/T, 5x | Shake 3 day |
| 10 mM histidine, 110 mM | 183 ± 29 | 247 ± 90 | 73 ± 21 |
| arginine, 1.5% sorbitol, | 133 ± 29 | 177 ± 74 | 57 ± 15 |
| 0.01% (w/v) PS20, pH 6.0 | 70 ± 26 | 63 ± 15 | 27 ± 6 |
| 20 mg/mL | 23 ± 15 | 10 ± 10 | 10 ± 0 |
| | 3 ± 6 | 0 ± 0 | 0 ± 0 |
| 10 mM histidine, 75 mM | 63 ± 35 | 90 ± 20 | 60 ± 10 |
| arginine, 3% sorbitol, | 40 ± 20 | 57 ± 21 | 47 ± 6 |
| 0.01% (w/v) PS20, pH 6.3 | 20 ± 17 | 33 ± 15 | 20 ± 17 |
| 20 mg/mL | 10 ± 10 | 17 ± 15 | 17 ± 12 |
| | 0 ± 0 | 0 ± 0 | 3 ± 6 |
| 10 mM histidine, 75 mM | 130 ± 62 | 173 ± 55 | 87 ± 21 |
| arginine, 3% sorbitol, | 73 ± 25 | 117 ± 50 | 63 ± 15 |
| 0.01% (w/v) PS20, pH 6.5 | 43 ± 25 | 40 ± 20 | 33 ± 6 |
| 50 mg/mL | 10 ± 10 | 10 ± 10 | 10 ± 10 |
| | 0 ± 0 | 0 ± 0 | 3 ± 6 |
| 10 mM histidine, 75 mM | 37 ± 15 | 127 ± 46 | 73 ± 31 |
| arginine, 3% sorbitol, | 30 ± 10 | 87 ± 25 | 60 ± 26 |
| 0.01% (w/v) PS20, pH 6.5 | 13 ± 6 | 40 ± 17 | 33 ± 15 |
| 20 mg/mL | 3 ± 6 | 17 ± 15 | 13 ± 15 |
| | 0 ± 0 | 0 ± 0 | 0 ± 0 |
| 10 mM histidine, 55 mM | 67 ± 29 | 263 ± 64 | 80 ± 26 |
| arginine, 55 mM glutamic | 37 ± 15 | 163 ± 21 | 67 ± 31 |
| acid, 3% sorbitol, | 23 ± 15 | 50 ± 17 | 37 ± 6 |
| 0.01% (w/v) PS20, pH 6.5 | 7 ± 12 | 7 ± 12 | 17 ± 6 |
| 20 mg/mL | 0 ± 0 | 0 ± 0 | 7 ± 12 |

### Example 11

Size exclusion chromatography (also known as gel filtration chromatography) separates the molecular forms of a protein based on their size. In this method, larger molecular species (e.g. aggregates, IgG dimers and oligomers) elute earlier than the desired (monomeric) IgG species as pre-peaks. Smaller molecular species (e.g. degradation products and fragments) elute later as post-peaks. SE-UPLC was performed on the anti-APRIL (VH14_1G.VL15) antibody stability samples according to the CMC13415 Purity of Items #5-0091 by SE-UPLC method, using a Waters Acquity UPLC BEH 200 SEC, 1.7 μm, 4.6×300 mm column and 10 mM phosphate, 0.4 M NaCl, pH7.0 mobile phase.

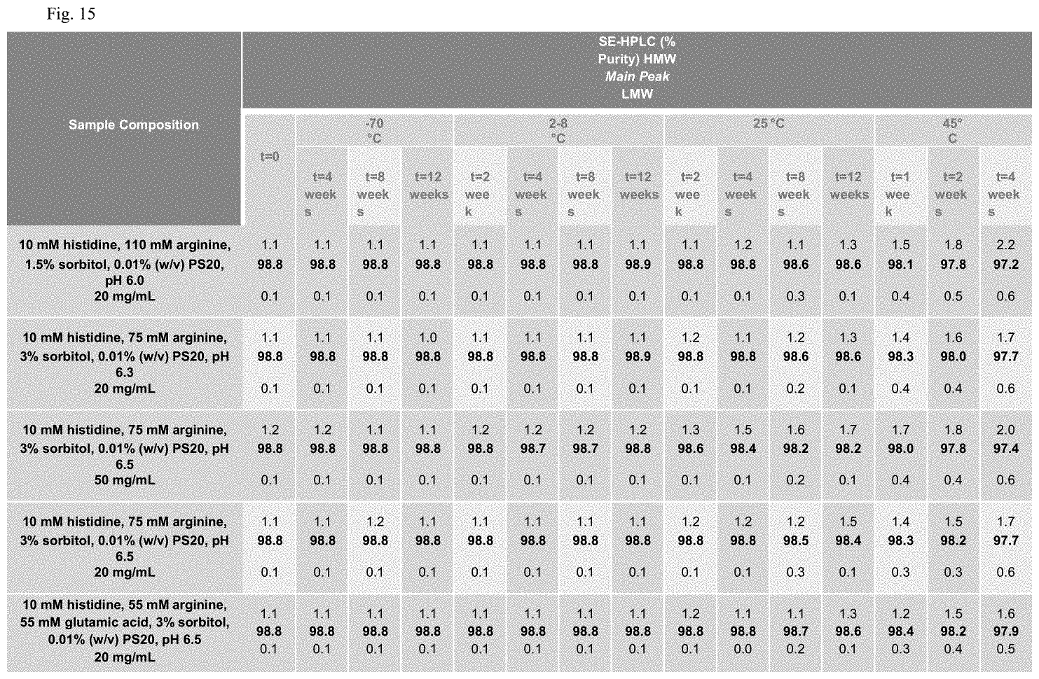

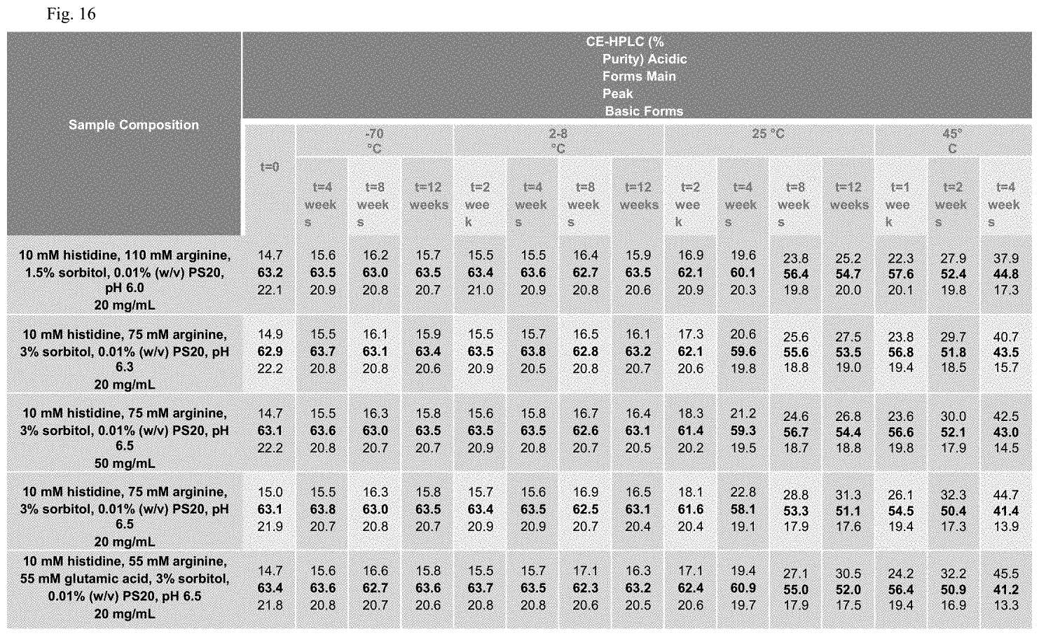

Following 1, 2, and 4 weeks of storage at 45° C., the purity results demonstrated the least stability of the formulation containing arginine/sorbitol at pH 6.0 compared to other three formulations with an equal concentration of 20 mg/mL (FIG. 15). Following 25° C. and 45° C. incubation, a comparison between the two formulations containing arginine/sorbitol pH 6.5 at 20 mg/mL or 50 mg/mL anti-APRIL antibody indicated a concentration dependent stability with the higher concentration formulation at 50 mg/mL having less stability. No significant changes were observed between any of the test formulations follow 12 weeks of storage at −70° C. and 2-8° C.

No significant changes were observed between any of the test formulations when exposed to freeze/thaw and 2-8° C. shake stress conditions as the resulting data was comparable to t=0 results and within assay variability (Table 26).

TABLE 26
SE-HPLC: Tabular results of formulations following freeze/thaw
and shaking stress.

| | SE-HPLC (% Purity) | | |
|---|---|---|---|
| | HMW | | |
| | Main Peak | | |
| | LMW | | |
| Sample Composition | t = 0 | F/T, 5x | Shake 3 day |
| 10 mM histidine, 110 mM | 1.1 | 1.1 | 1.1 |
| arginine, 1.5% sorbitol, | 98.8 | 98.7 | 98.8 |
| 0.01% (w/v) PS20, pH 6.0 | 0.1 | 0.1 | 0.1 |
| 20 mg/mL | | | |
| 10 mM histidine, 75 mM arginine, | 1.1 | 1.1 | 1.1 |
| 3% sorbitol, | 98.8 | 98.8 | 98.8 |
| 0.01% (w/v) PS20, pH 6.3 | 0.1 | 0.1 | 0.1 |
| 20 mg/mL | | | |
| 10 mM histidine, 75 mM arginine, | 1.2 | 1.2 | 1.2 |
| 3% sorbitol, | 98.8 | 98.8 | 98.7 |
| 0.01% (w/v) PS20, pH 6.5 | 0.1 | 0.1 | 0.1 |
| 50 mg/mL | | | |
| 10 mM histidine, 75 mM arginine, | 1.1 | 1.1 | 1.1 |
| 3% sorbitol, | 98.8 | 98.8 | 98.8 |
| 0.01% (w/v) PS20, pH 6.5 | 0.1 | 0.1 | 0.1 |
| 20 mg/mL | | | |
| 10 mM histidine, 55 mM arginine, | 1.1 | 1.1 | 1.1 |
| 55 mM glutamic acid, 3% | 98.8 | 98.8 | 98.8 |
| sorbitol, 0.01% (w/v) PS20, pH | 0.1 | 0.1 | 0.1 |
| 6.5, 20 mg/mL | | | |

### Example 12

Ion exchange chromatography separates molecules based on differences in their accessible surface charges. Binding is dependent on the ionic attraction between molecules of the opposite electric charge. The analysis applied herein utilized a weak cation exchange (CE-HPLC) column. Elution is accomplished with a gradient of increasing pH. The pH gradient separations are performed using a high performance liquid chromatography system (Agilent 1260) equipped with a UV detector monitoring at 280 nm (A280) and integration software (Chemstation ver. C.01.07 software package). A Dionex ProPac WCX-10 column (4.0×250 mm) was used in this application to resolve and provide a profile of measurable populations of anti-APRIL antibody charged species over the course of the stability study described herein.

Following the incubation at 25° C. and 45° C., main peak and basic peak levels were observed to decrease over the duration of the stability study resulting in an increase of acidic peaks for all the formulations. These differences were observed to be lower at pH 6.0 and pH 6.3 than the higher pH 6.5 for formulations. Following the incubation at 25° C. and 45° C., a concentration dependent stability was observed in which greater stability was observed for the 50 mg/mL formulation as compared to the same formulation containing 20 mg/mL anti-APRIL (VH14_1G.VL15) antibody. This is interestingly different than the results determined by SE-UPLC. No significant changes were observed between any of the test formulations follow 12 weeks of storage at −70° C. and 2-8° C. or upon exposure to freeze/thaw and 2-8° C. shake stress conditions as the resulting data was comparable to t=0 results and within assay variability (Table 27).

TABLE 27
CE-HPLC: Tabular results of formulations following freeze/thaw
and shaking stress.

| | CE-HPLC (% Purity) | | |
|---|---|---|---|
| | Acidic Forms Main | | |
| | Peak | | |
| | Basic Forms | | |
| Sample Composition | t = 0 | F/T, 5x | Shake 3 day |
| 10 mM histidine, 110 mM | 14.7 | 14.6 | 14.2 |
| arginine, 1.5% sorbitol, | 63.2 | 63.8 | 63.9 |
| 0.01% (w/v) PS20, pH 6.0 | 22.1 | 21.6 | 21.9 |
| 20 mg/mL | | | |
| 10 mM histidine, 75 mM arginine, | 14.9 | 14.8 | 14.0 |
| 3% sorbitol, | 62.9 | 63.8 | 64.1 |
| 0.01% (w/v) PS20, pH 6.3 | 22.2 | 21.5 | 21.9 |
| 20 mg/mL | | | |
| 10 mM histidine, 75 mM arginine, | 14.7 | 14.8 | 14.4 |
| 3% sorbitol, | 63.1 | 63.8 | 63.8 |
| 0.01% (w/v) PS20, pH 6.5 | 22.2 | 21.4 | 21.9 |
| 50 mg/mL | | | |
| 10 mM histidine, 75 mM arginine, | 15.0 | 14.7 | 14.1 |
| 3% sorbitol, | 63.1 | 63.9 | 64.1 |
| 0.01% (w/v) PS20, pH 6.5 | 21.9 | 21.4 | 21.8 |
| 20 mg/mL | | | |
| 10 mM histidine, 55 mM arginine, | 14.7 | 14.7 | 14.2 |
| 55 mM glutamic acid, 3% | 63.4 | 64.0 | 63.9 |
| sorbitol, 0.01% (w/v) PS20, pH | 21.8 | 21.3 | 21.9 |
| 6.5, 20 mg/mL | | | |

### Example 13

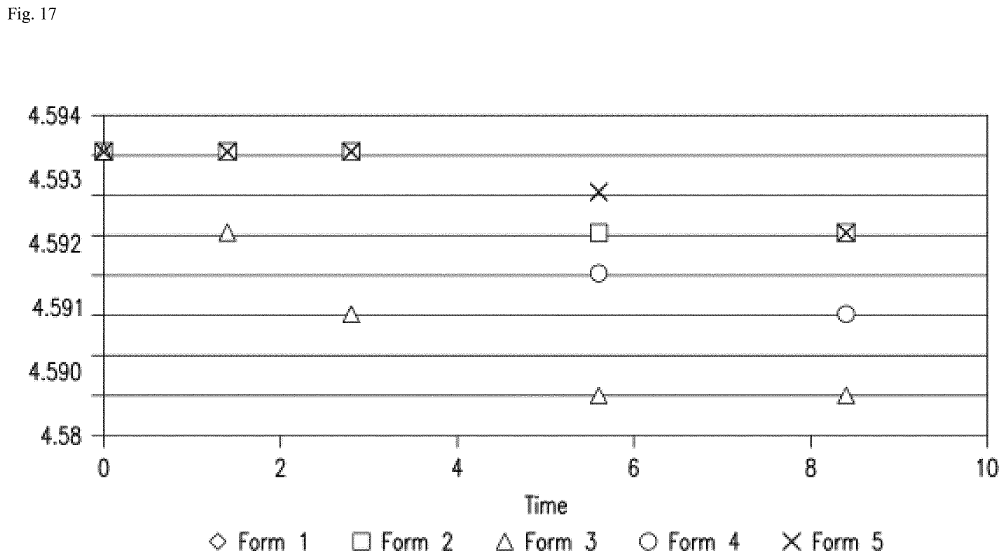

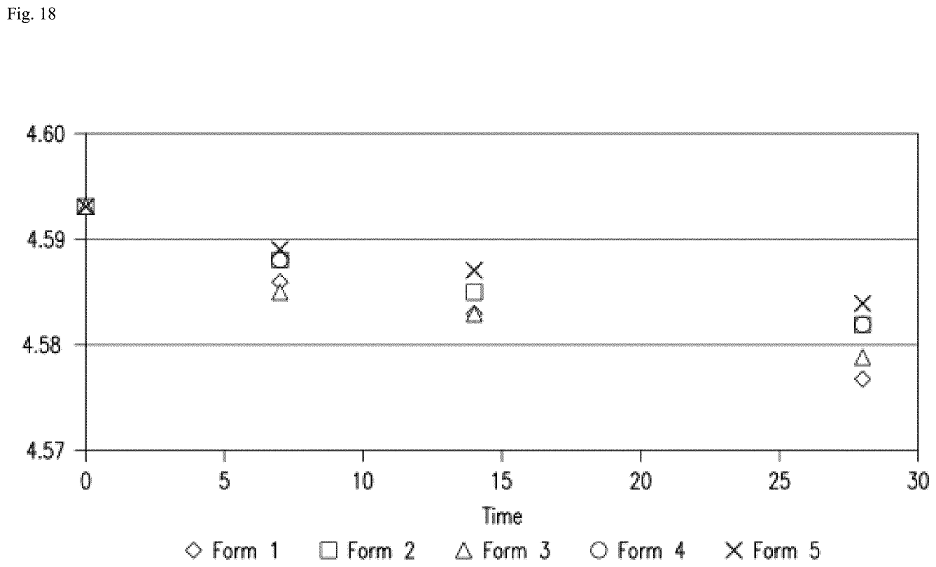

To approach data analysis in a more rigorous fashion, rate constants were determined for the loss of anti-APRIL (VH14_1G.VL15) antibody purity for each formulation and temperature condition to approximate first-order degradation kinetics. For the 25° C. and 45° C. testing condition, a plot of ln purity versus time was made using the percent VH14_1G.VL15 purity values found in the SEC-HPLC analysis. The results are graphically represented in FIGS. 17 and 18. Straight lines generated using the data indicate pseudo first-order degradation kinetics, from which the slope is correlated to a rate constant, (−kobs) for degradation (Table 28).

TABLE 28
Vehicle and temperature dependence on kobs.

| | 45° C. | | 25° C. | |
|---|---|---|---|---|
| | k_{obs} | R² | k_{obs} | R² |
| Form 1 | 5.54E−04 | 0.95292 | 2.99 × 10⁻⁵ | 0.82759 |
| Form 2 | 3.82E−04 | 0.91694 | 2.99 × 10⁻⁵ | 0.82759 |
| Form 3 | 4.66E−04 | 0.86303 | 7.25 × 10⁻⁵ | 0.85329 |
| Form 4 | 3.74E−04 | 0.94912 | 5.56 × 10⁻⁵ | 0.89855 |
| Form 5 | 3.11E−04 | 0.94055 | 2.56 × 10⁻⁵ | 0.90567 |

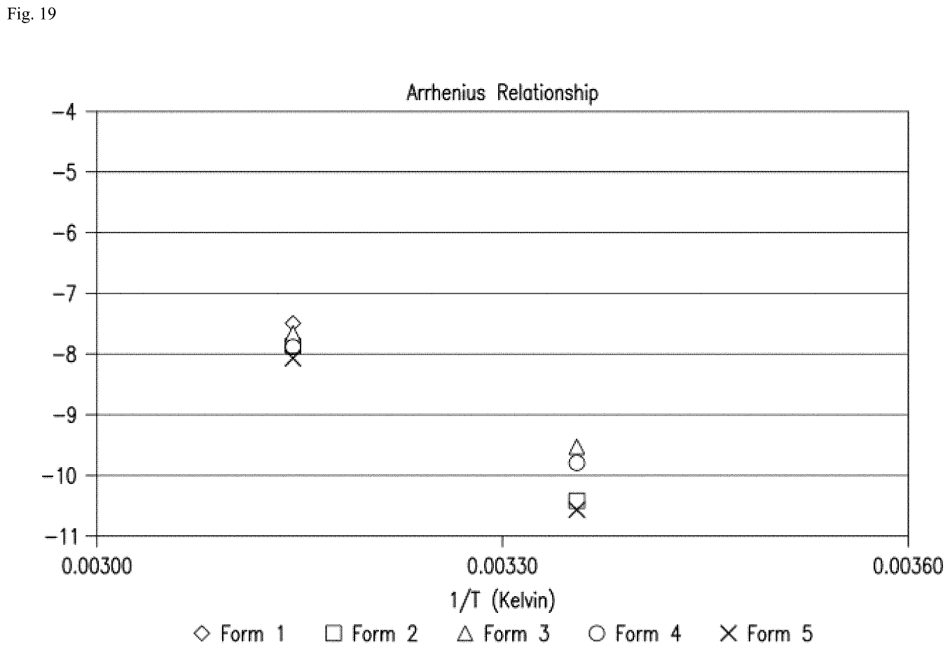

Using these data, Arrhenius plots (ln kobs versus 1/T) were generated for each of the anti-APRIL (VH14_1G.VL15) antibody formulations at the three stability temperatures (FIG. 19). The fitted parameters of line were obtained (Table 29). Note usually at least three temperatures are used in an Arrhenius plot, however as no loss in purity was observed after 12 weeks at 2-8° C., this was not possible in this case. A good linear fit of ln k_{obs} versus 1/T indicates a similar decomposition mechanism operating within the temperature ranges tested. Furthermore, this implies that decomposition follows Arrhenius behavior.

TABLE 29
Fitted parameters resulting from linear regression analysis
of the ln kobs vs. 1/T for each formulation.

| Formulation | Slope | Intercept | R² |
|---|---|---|---|
| Form 1 | −13840.4 | 36.00 | 1 |
| Form 2 | −12079.6 | 30.10 | 1 |
| Form 3 | −8823.9 | 20.06 | 1 |
| Form 4 | −9042.6 | 20.53 | 1 |
| Form 5 | −11853.4 | 29.18 | 1 |

From the Arrhenius relationship of Equation 1, the data was then used to calculate ln k_{obs} for each vehicle at 5° C., the t_{95%} was calculated which corresponds to days of incubation until anti-APRIL (VH14_1G.VL15) antibody purity would approach 95% of the total sample composition (Table 30). Solution stability was calculated to be on the order of >4600 days—or approximately >12 years—for all of the formulations when stored at 5° C.

TABLE 30
Approximate rates of degradation for VH14_1G.VL15
and t_{95%} for storage at 5° C.

| | | | T_{95%}, | |
|---|---|---|---|---|
| | | | day at | |
| | | | which 95% | Observed |
| | | | sample | % purity |
| | In | | purity is | at 84 |
| Formulation | k_{obs} | k_{obs} | obtained | days |
| Form 1 | −13.75 | 1.06 × 10⁻⁶ | 37405 | 98.9 |
| 10 mM histidine, | | | | |
| 110 mM arginine, | | | | |
| 1.5% sorbitol, 0.01% | | | | |
| (w/v) PS20, | | | | |
| pH 6.0, 20 mg/mL | | | | |
| Form 2 | −13.33 | 1.63 × 10⁻⁶ | 24463 | 98.9 |
| 10 mM histidine, | | | | |
| 75 mM arginine, | | | | |
| 3% sorbitol, 0.01% | | | | |
| (w/v) PS20, | | | | |
| pH 6.3, 20 mg/mL | | | | |
| Form 3 | −11.66 | 8.64 × 10⁻⁶ | 4605 | 98.8 |
| 10 mM histidine, | | | | |
| 75 mM arginine, | | | | |
| 3% sorbitol, 0.01% | | | | |
| (w/v) PS20, | | | | |
| pH 6.5, 50 mg/mL | | | | |
| Form 4 | −11.98 | 6.28 × 10⁻⁶ | 6337 | 98.8 |
| 10 mM histidine, | | | | |
| 75 mM arginine, | | | | |
| 3% sorbitol, 0.01% | | | | |
| (w/v) PS20, | | | | |
| pH 6.5, 20 mg/mL | | | | |
| Form 5 | −13.43 | 1.47 × 10⁻⁶ | 27122 | 98.8 |
| 10 mM histidine, | | | | |
| 55 mM arginine, | | | | |
| 55 mM glutamic acid, | | | | |
| 3% sorbitol, | | | | |
| 0.01% (w/v) PS20, | | | | |
| pH 6.5 20 mg/mL | | | | |

### Example 14

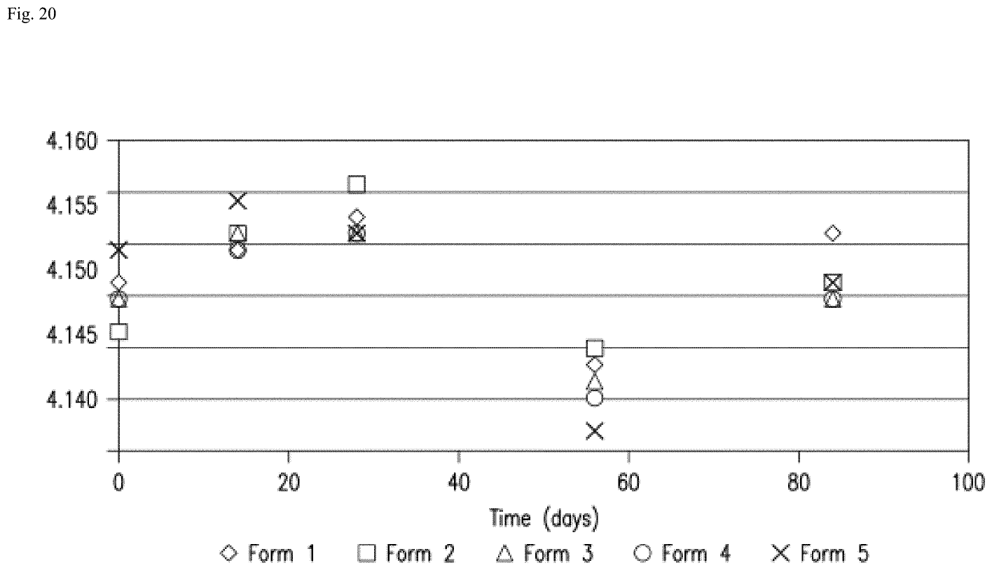

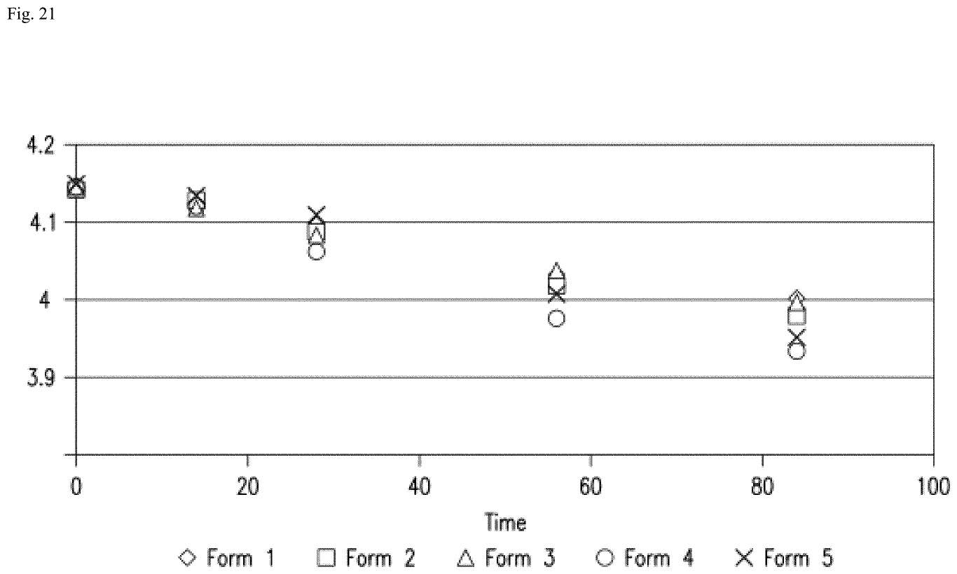

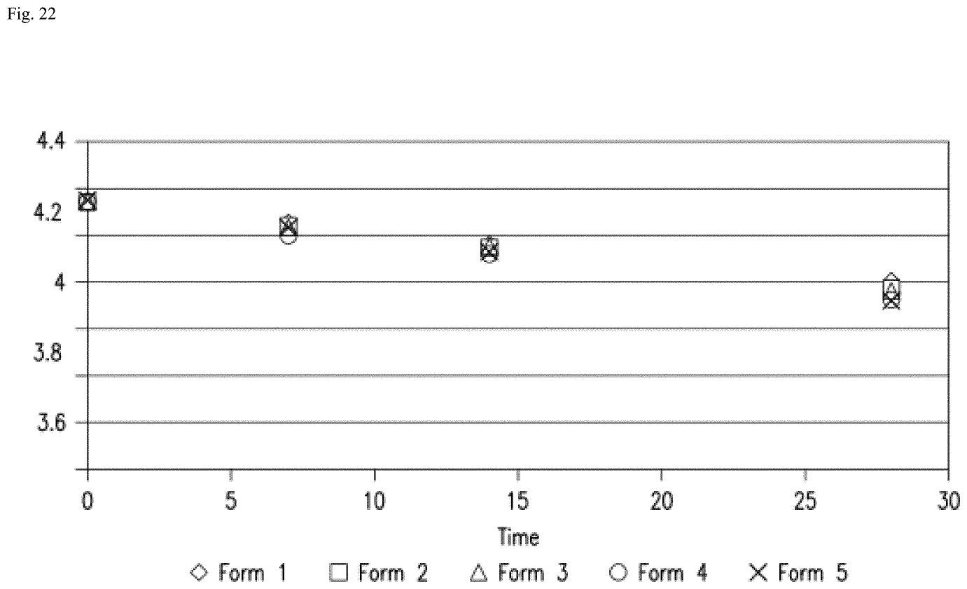

Using the same approach, one can estimate sample purity out to 95% of the original value using the collected CE-HPLC data. For each testing condition, a plot of ln purity versus time was made using the percent anti-APRIL (VH14_1G.VL15) antibody purity values found in the CECHPLC analysis. Straight lines generated using the data of FIGS. 20-22 indicate pseudo first-order degradation kinetics, at least for the 45° C. and 25° C. test samples, from which the slope is correlated to a rate constant (−k_{obs}) for degradation (Table 31).

TABLE 31
Vehicle and temperature dependence on k_{obs}:

| | 45° C. | | 25° C. | | 5° C. | |
|---|---|---|---|---|---|---|
| | k_{obs} | R² | k_{obs} | R² | k_{obs} | R² |
| Form 1 | 1.23E−02 | 0.99754 | 1.82E−03 | 0.98238 | 1.19 × 10⁻⁵ | 0.00504 |
| Form 2 | 1.31E−02 | 0.99874 | 2.06E−03 | 0.98201 | 3.01 × 10⁻⁵ | 0.02379 |
| Form 3 | 1.35E−02 | 0.99855 | 1.76E−03 | 0.99138 | 7.71 × 10⁻⁵ | 0.19475 |
| Form 4 | 1.46E−02 | 0.98871 | 2.66E−03 | 0.97620 | 7.62 × 10⁻⁵ | 0.17237 |
| Form 5 | 1.53E−02 | 0.99947 | 2.53E−03 | 0.97648 | 1.35 × 10⁻⁴ | 0.27511 |

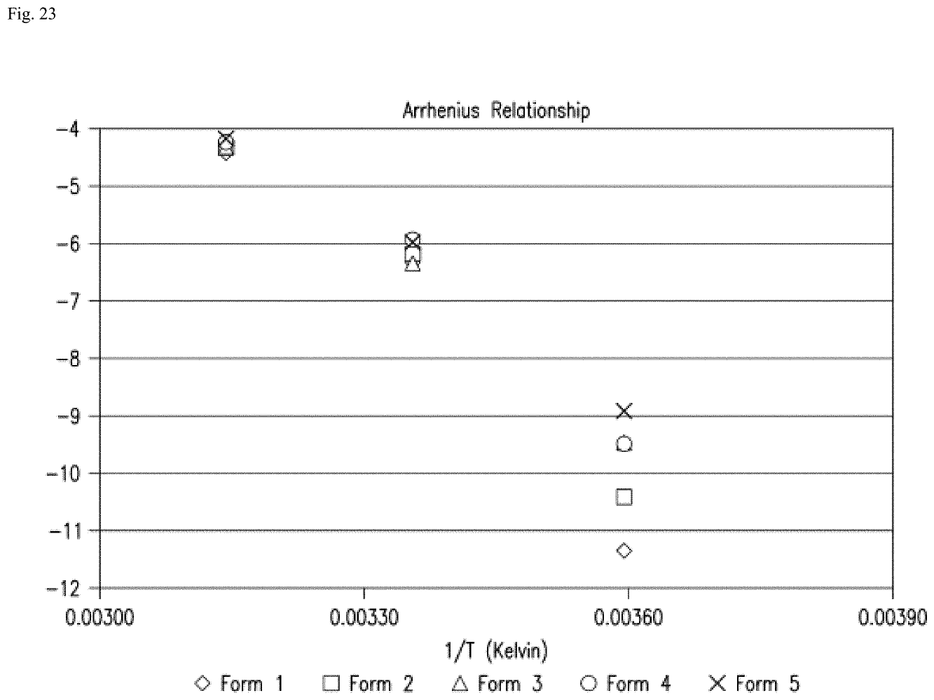

Using this data, Arrhenius plots (ln k_{obs} versus 1/T) were generated for each of the formulations at the three stability temperatures (FIG. 23). From this analysis, the fitted parameters of line were obtained indicating a good degree of correlation within the data, i.e., R² values≥0.95. A good linear fit of ln k_{obs} versus 1/T indicates a similar decomposition mechanism operating within the temperature ranges tested (Table 32). Furthermore, this implies that decomposition follows Arrhenius behavior.

TABLE 32
Fitted parameters resulting from linear regression analysis
of the ln kobs vs. 1/T for each formulation:

| Formulation | Slope | Intercept | R² |
|---|---|---|---|
| Form 1 | −15484.4 | 44.74 | 0.9542 |
| Form 2 | −13540.5 | 38.57 | 0.9666 |
| Form 3 | −11471.0 | 31.89 | 0.9932 |
| Form 4 | −11701.0 | 32.82 | 0.9740 |
| Form 5 | −10510.4 | 29.00 | 0.9902 |

From the Arrhenius relationship, in which slope=−Ea/R and intercept=ln A, the data from Table 32 was then used to calculate ln kobs for each vehicle at 5° C. Moreover, from kobs at 5° C. and using a linear form of the rate equation for first order kinetics where k is an observed rate constant (kobs) and Purity₀ and Purityₜ are the purity at time 0 and t, respectively, the t_{95%} was calculated which corresponds to days of incubation until antibody purity would approach 95% of the original sample purity (Table 33). Antibody stability was calculated to be on the order of >1200 days—or approximately >3 years—in the formulation at pH 6.3 and even longer at pH 6.0.

TABLE 33
Approximate rates of degradation and t_{95%} for storage at 5° C.:

| | | | t_{95%}, | |
|---|---|---|---|---|
| | | | day at | |
| | | | which 95% | |
| | | | of original | Observed |
| | | | sample | % purity |
| | | | purity is | at 84 |
| Formulation | In k_{obs} | k_{obs} | obtained | days |
| Form 1 | −10.93 | 1.80 × 10⁻⁵ | 2855 | 63.5 |
| 10 mM histidine, | | | | |
| 110 mM arginine, | | | | |
| 1.5% sorbitol, | | | | |
| 0.01% (w/v) PS20, | | | | |
| pH 6.0 20 mg/mL | | | | |
| Form 2 | −10.11 | 4.08 × 10⁻⁵ | 1256 | 63.2 |
| 10 mM histidine, | | | | |
| 75 mM arginine, | | | | |
| 3% sorbitol, 0.01% | | | | |
| (w/v) PS20, pH 6.3 | | | | |
| 20 mg/mL | | | | |
| Form 3 | −9.35 | 8.66 × 10⁻⁵ | 593 | 63.1 |
| 10 mM histidine, | | | | |
| 75 mM arginine, | | | | |
| 3% sorbitol, 0.01% | | | | |
| (w/v) PS20, pH 6.5 | | | | |
| 50 mg/mL | | | | |
| Form 4 | −9.25 | 9.62 × 10⁻⁵ | 533 | 63.1 |
| 10 mM histidine, | | | | |
| 75 mM arginine, | | | | |
| 3% sorbitol, 0.01% | | | | |
| (w/v) PS20, pH 6.5 | | | | |
| 20 mg/mL | | | | |
| Form 5 | −8.79 | 1.53 × 10⁻⁴ | 335 | 63.2 |
| 10 mM histidine, | | | | |
| 55 mM arginine, | | | | |
| 55 mM glutamic | | | | |
| acid, 3% sorbitol, | | | | |
| 0.01% (w/v) PS20, | | | | |
| pH 6.5 20 mg/mL | | | | |

Target concentrations of 20 mg/mL or 50 mg/mL were obtained in all of the test anti-APRIL (VH14_1G.VL15) antibody formulations resulting in clear, colorless and particulate-free formulations throughout the duration of the study. No significant changes in monomer or charged species were observed between any of the test formulations follow 12 weeks of storage at −70° C. and 2-8° C., freeze/thaw, or 2-8° C. shake stress conditions as the resulting data was comparable to t=0 results and within assay variability. Turbidity values following 25° C. and 45° C. incubation indicated the greatest stability of the formulation with arginine and sorbitol at pH 6.3. As determined by SE-HPLC, higher VH14_1G.VL15 stability was maintained in samples formulated in the pH 6.3 to 6.5 range as compared to pH 6.0, which was only clearly observable at the 45° C. test condition. Greater stability was observed at the 20 mg/mL protein concentration as compared to the same formulation at 50 mg/mL anti-APRIL (VH14_1G.VL15) antibody. Following the incubation at 25° C. and 45° C., CE-HPLC evaluation on charged species revealed the preference for formulations with a pH of 6.0 or pH 6.3 having smaller changes of all the charged species. Based upon calculated degradation rates using the Arrhenius relationship, predicted monomer and charged species stability for the 10 mM histidine, 75 mM arginine, 3% sorbitol, 0.01% (w/v) PS20, pH 6.3 formulation is on the order of at least three years at 5° C.

### Example 15

In a clinical study (see PCT/IB2020/000020), subjects with multiple myeloma received anti-APRIL antibody BION-1301 (also referred to herein as VH14_1G.VL15) at doses up to 1350 mg IV once per week or up to 2700 mg IV given once every 2 weeks. BION-1301 inhibited serum levels of APRIL in a dose-dependent manner at doses between 50 to 2700 mg. At 450 mg, 95% target engagement (TE) was achieved around peak exposure levels; at 1350 mg, 95% TE was maintained throughout the dosing interval. Exposure was approximately dose-linear over the dose range evaluated, with a low incidence of anti-drug antibodies. Overall, the antibody was well tolerated with no dose limiting toxicities reported, and a maximum tolerated dose was not determined. A single serious adverse reaction (SAR) was reported (wheezing) in a patient at the 50 mg dose and resulted in treatment discontinuation.

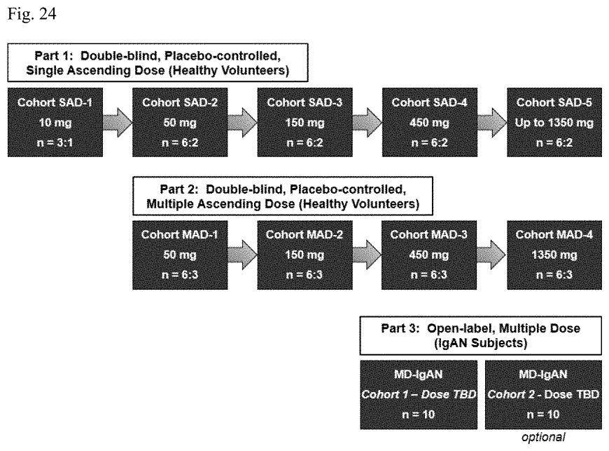

The following is a Phase 1 study in volunteers receiving BION-1301 at single doses up to 1350 mg IV and multiple doses up to 450 mg IV to evaluate the safety, tolerability, PK, and PD of intravenously administered BION-1301. The study has started in HVs and will also be conducted in adults with IgAN. This study design includes 3 parts as depicted in the study schema (FIG. 24). BION-1301 is supplied as a solution intended for intravenous (IV) administration. BION-1301 will be diluted and administered at the assigned dose level by IV infusion over approximately 2 hours. The BION-1301 is supplied in a vial at 20 mg/mL in at least 5 mL of solution, which will be diluted with 0.9% saline solution prior to infusion. For example, the number of vials need for a particular dose is determined as volume required=mg dose/20 mg/mL, and this volume is divided by 5 mL/vial to determine the total number of vials needed. If a 250 mL 0.9% saline bag is used for dilution, a volume equal to the amount of antibody solution needed for the dosing will be removed from the saline bag prior to adding the antibody solution. The amount of antibody solution needed is removed from each vial (single use vials, with fresh syringe for each vial) and added to the bag of saline. For Part 3: following the initial dose, the infusion time may be reduced to 1 hour for subsequent doses, provided there are no tolerability issues, as assessed by the Investigator. Placebo is a 0.9% normal saline solution intended for IV administration. Placebo will be administered by IV infusion over approximately 2 hours (applicable to Parts 1 and 2 only).

TABLE 34
BION-1301 formulation for IV administration.

| | BION-1301 | Placebo |
|---|---|---|
| Dosage | Concentrate for solution for | Solution for |
| Formulation | infusion 20 mg/mL in 10 mM | infusion |
| | L-histidine, 75 mM L-arginine, | |
| | 3% sorbitol, and 0.01% (w/w) | |
| | polysorbate 20, pH 6.3. | |
| Unit Dose | 10 mg, 50 mg, 150 mg, | 0.9% normal |
| | 450 mg, 1350 mg | Saline |
| Strength/ | 20 mg/mL, which will be diluted | NA |
| Concentration | with 0.9% normal saline prior to | |
| | administration as per Pharmacy | |
| | Manual (as described above) | |
| Route of | IV infusion | IV infusion |
| Administration | | |

IV = intravenous;
NA = not applicable;
SAD-HV = single ascending dose in healthy volunteers

| Objective | Endpoint |
|---|---|
| Primary | |
| Characterize the safety and | Incidence and severity of treatment-emergent |
| tolerability of BION-1301 | adverse events (TEAEs), treatment-emergent |
| in healthy volunteers (HVs | serious adverse events (SAEs), and changes |
| and in patients with IgAN | from baseline in safety parameters |
| Secondary | |
| Characterize the | Serum concentration-time profiles and |
| pharmacokinetics (PK) of | noncompartmental PK parameters |
| BION-1301 administered by | |
| IV infusion or SC injection | |
| following a single dose | |
| and/or multiple doses | |
| Assess the immunogenicity | Incidence of anti-drug antibodies (ADA) |
| of BION-1301 following | and neutralizing antibodies (NAbs) |
| a single dose and | |
| multiple doses | |
| Characterize the | Changes from baseline in immunoglobulin |
| pharmacodynamic (PD) | levels (IgA, IgG, IgM) |
| impact of BION-1301 on | |
| immunoglobulins | |
| following a single dose | |
| and multiple doses | |
| Assess changes in pro- | Changes from baseline in urinary protein/ |
| teinuria and renal function | creatine ratio (UPCR) and urinary protein |
| in patients with IgAN | excretion based on 24-hour urine collection. |
| Exploratory | |
| To evaluate the optimal dose | The optimal dose and schedule of BION- |
| and schedule of BION-1301 | 1301 will be assessed based on all |
| to achieve a clinically | available PK, PD, biomarker, and |
| meaningful on target effect | preliminary efficacy data. |
| in patients with IgAN. | |
| Assess changes in renal | Changes from baseline in: |
| function in patients with | urinary albumin/creatinine ratio (UACR) |
| IgAN | and urinary albumin excretion based |
| | on 24-hour urine collection |
| | estimated glomerular filtration rate (eGFR) |
| Assess the relationship of | Correlation of DNA variability (such |
| DNA variability with safety, | as single nucleotide polymorphisms |
| tolerability and/or efficacy | [SNPs]) and its relationship with safety, |
| | tolerability and/or efficacy, if indicated |
| Characterize select | Changes from baseline in: |
| biomarkers of BION-1301 | APRIL levels |
| activity following a | Galactose-deficient (gd)-IgA1 and anti-gd- |
| single dose and multiple | IgA1 antibodies when feasible |
| doses | Other exploratory biomarker variables |

Part 1 (SAD-HV) was a double-blind, randomized, placebo-controlled single ascending dose (SAD) design in HVs. Up to 5 dose cohorts may be evaluated; the dose levels were 10 mg, 50 mg, 150 mg, 450 mg and 1350 mg. HVs within each cohort were randomized in a 3:1 ratio to receive either BION-1301 or placebo, respectively. Sentinel dosing was employed within each cohort to confirm whether there are any safety concerns that warrant discontinuation of dosing in the remaining HVs in the cohort.

Part 2 (MAD-HV) was double-blind, randomized, placebo-controlled multiple ascending dose (MAD) design conducted in HVs. HVs within each cohort were randomized in a 2:1 ratio to receive either BION-1301 or placebo, respectively. Each HV received BION-1301 or placebo administered by IV infusion once every 2 weeks (Q2W) for a total of 3 doses (ie, Day 1, Day 15, and Day 29). Up to four dose cohorts may be evaluated; the anticipated per dose levels are 50 mg, 150 mg, 450 mg, and 1350 mg.

In Part 1 and Part 2, dose escalation was stopped if any of the following criteria were met:
One or more subjects in a cohort develop an SAE assessed by the Investigator as related to BION-1301;
Two or more subjects in a cohort experience severe non-serious AEs (≥Grade 3 per National Cancer Institute Common Terminology Criteria for Adverse Events [NCI-CTCAE] Version 5.0) assessed by the Investigator as related to BION-1301;
Two or more subjects in a cohort receiving study drug develop similar clinically significant laboratory, ECG, or vital sign abnormalities in the same organ class, indicating dose-limiting intolerance;
An individual AUC(0-168h) and/or Cmax that exceeds the mean AUC (24,000 μg*day/mL) or mean Cmax (5720 μg/mL) established as NOAEL in cynomolgus monkeys; or
Two or more subjects in the same cohort develop IgG levels persistently below 1.5 g/L, or below 3.0 g/L with signs of bacterial infection.

Part 3 (MD-IgAN) is an ongoing open-label multiple dose design in adult subjects with IgAN. MD-IgAN will initiate after the last MAD-HV cohort has been evaluated by the Safety Review Team (SRT). Part 3 will consist of at least 2 cohorts with the option to add additional cohorts if alternative doses or dosing schedules are explored. The total duration of dosing Cohorts 1, 2, and beyond (if applicable) is up to 2 years. The sample size of Cohort 1 will be approximately 10 IgAN patients. For Cohort 2 and any additional cohorts that are added, the sample size will not exceed approximately 40 IgAN patients, total.

The first cohort will receive BION-1301 at a dose level of 450 mg that will be given by IV infusion every 2 weeks for up to 1 year. The second cohort will initiate enrollment after SRT recommendation and review of available data from a minimum of 5 subjects in the MD-IgAN Cohort 1 demonstrate adequate safety and tolerability. When the SRT recommends initiating Cohort 2, all patients will receive BION-1301 via SC injection (formulated at 150 mg/mL in 10 mM L-histidine, 75 mM L-arginine, 3% sorbitol, and 0.01% (w/w) polysorbate 20, pH 6.3) for up to 1 year. Selection of the dose and dosing regimen for MD-IgAN Cohort 2 will be based on all available safety, PK, and PD data from all prior subjects, including subjects from MD-IgAN Cohort 1. In the event the SRT recommends a change of dose or schedule for upcoming subjects, a new cohort will be formed. One or more additional cohorts may be explored beyond Cohorts 1 and 2. The maximum dose of BION-1301 in Part 3 will not exceed 1350 mg, and the maximum exposure of BION-1301 over the dosing interval will not exceed that observed in a Phase 1 dose escalation trial conducted in subjects with multiple myeloma (ADU-CL-16; NCT03340883). For subjects receiving BION-1301 via SC injection, the dose will not exceed 600 mg QW. All patients who receive BION-1301 via IV infusion will be required to transition to the SC administration route after at least 24 weeks of IV dosing. The dose or schedule of patients switching from IV to SC may be modified to account for bioavailability differences between administration routes and will be determined by the SRT in collaboration with the sponsor based on all available data.

In the event of dose escalation in Part 3, dose escalation will stop if two or more subjects in a cohort experience drug-related toxicity or TEAEs that, in the opinion of the SRT, would preclude further dosing of subjects, given what might be considered acceptable risks for the study population.

Inclusion Criteria: Healthy Volunteers
1. Healthy male or female volunteers between the ages of 18 and 55 years old, inclusive, at screening
2. Females must be of non-childbearing potential (per CTFG 2014)
3. Males must agree to follow the protocol-specified contraception guidance
4. Body mass index (BMI) between 18 and 35 kg/m², inclusive, at Screening with a weight of at least 50 kg
5. Non-smoker, defined as an individual who has not smoked previously and/or who has discontinued smoking or the use of nicotine/nicotine-containing products (including snuff, e-cigarettes and similar products) at least 3 months before the Screening Visit, confirmed by medical history
6. In good health, as determined by medical history, physical examination, vital signs assessment, 12-lead electrocardiogram (ECG) and clinical laboratory evaluations within normal reference ranges (or outside of normal reference ranges considered not clinically relevant by the Investigator)
7. Total IgG>10 g/L at Screening
8. Able to provide signed informed consent which includes compliance with the requirements and restrictions listed in the informed consent form (ICF)

Exclusion Criteria: Healthy Volunteers
Healthy volunteers who meet any of the following exclusion criteria will not be eligible to participate:
1. Known or suspected allergy or hypersensitivity to any component of BION-1301, or history of severe hypersensitivity reaction to any monoclonal antibody
2. Regular consumption of alcohol within 6 months prior to Screening (>7 drinks/week for females, >14 drinks/week for males where 1 drink=5 ounces [150 mL] of wine or
   12 ounces [360 mL] of beer or 1.5 ounces [45 mL] of hard liquor), or use of soft drugs (such as marijuana) within 3 months prior to the Screening Visit, or hard drugs (such as cocaine and phencyclidine) within 1 year prior to the Screening Visit and/or positive blood or urine test results for drugs of abuse or alcohol at the Screening Visit or Admission
3. Donated blood in the 3 months prior to the first dose of study drug, plasma in the 7 days prior to the first dose of study drug, or platelets in the 6 weeks prior to the first dose of study drug
4. Unable to abstain from strenuous exercise for 7 days before the first dose of study drug until the final Follow-up Visit
5. Used any non-prescribed systemic or topical medication, remedy or supplement, any prescribed systemic or topical medication (with the exception of either paracetamol to a maximum of 2 g per day or ibuprofen up to 800 mg per day or hormone replacement therapy [HRT]) within 28 days or 5 half-lives (whichever is longer) of first dose of study drug
6. Poor venous access for the purpose of multiple blood draws and study drug administration, as judged by the Investigator
7. Participated in any other study in which receipt of an investigational new drug, or investigational device occurred within 28 days, or 5 half-lives (whichever is longer) of first dose of study drug in the present study
8. Received an approved monoclonal antibody drug within 28 days, or 5 half-lives (whichever is longer) prior to the first dose of study drug in the present study
9. Any confirmed or suspected immunosuppressive or immune-deficient state, including human immunodeficiency virus (HIV) infection or asplenia; recurrent, severe infections and chronic immunosuppressant medication (more than 28 days for inhaled/topical corticosteroids, and more than 1 week for systemic immunosuppressive or corticosteroid therapy) within 6 months prior to Day 1
10. Positive serology test for hepatitis A virus IgM antibodies (anti-HAV IgM), hepatitis B surface antigen (HBsAg), hepatitis C virus (HCV) antibodies, or antibodies to HIV-1 and/or HIV-2 at Screening
11. Major surgery or significant traumatic injury occurring within 28 days prior to first dose of study drug. If major surgery occurred >28 days prior to first dose of study drug, individual must have recovered adequately from any toxicity and/or complications from the intervention prior to the first dose of study drug.
12. History or evidence of a clinically significant disorder, condition, or disease that could pose a risk to subject safety or interfere with the study, or would make the subject unsuitable for participation, eg, respiratory, renal, hepatic, gastrointestinal, hematological, lymphatic, neurological, cardiovascular, or psychiatric disease
13. Positive QuantiFERON-TB Gold Plus test at Screening
14. Received a live vaccination within 3 months prior to Screening or plan to have a live vaccination within 3 months after the last dose of study drug
15. Female who is breastfeeding or who has a positive serum pregnancy test at Screening or a positive urine pregnancy test on Day −1
16. Any other condition that, in the opinion of the Investigator or the Medical Monitor, renders the participant unsuitable for inclusion or could interfere with the participant's ability to complete the study

Inclusion Criteria for Patients with IgAN
Individuals eligible to participate must meet all of the following criteria:
1. Male or female≥18 years old at Screening
2. Women of child-bearing potential (WOCBP; per CTFG 2014) must agree to follow the protocol-specified contraception guidance throughout the study (from Screening through approximately 5 half-lives (165 days) after the final dose of study drug)
3. Males must agree to follow the protocol-specified contraception guidance throughout the study (from Screening through approximately 5 half-lives (165 days) after the final dose of study drug)
4. BMI between 18 and 40 kg/m², inclusive, at Screening with a weight of at least 50 kg
5. Considered eligible for this study, as determined by medical history, physical examination, vital signs assessment, 12-lead ECG, and clinical laboratory evaluations within normal reference ranges or consistent with IgAN (or if outside of normal reference ranges, considered not clinically relevant by the Investigator)
6. Diagnosis of IgAN verified by biopsy taken within the past 10 years
7. Urine protein≥0.5 g/24 h; OR UPCR≥0.5 g/g (or ≥50 mg/mmol) based on assessment of 24-hour urine collected at Screening.
8. eGFR (per Chronic Kidney Disease Epidemiology Collaboration [CKD-EPI] formula) or measured GFR>45 mL/min per 1.73 m²; OR 30-45 mL/min per 1.73 m² if kidney biopsy performed within 2 years prior to Day 1 does not provide evidence of glomerular fibrosis (e.g. S1 by Oxford classification).
9. Stable on an optimized dose of angiotensin converting enzyme (ACE) inhibitors and/or angiotensin-receptor blockers (ARBs) for at least 3 months prior to Screening or intolerant to ACE/ARB.
10. Able to provide signed informed consent which includes compliance with the requirements and restrictions listed in the ICF
11. Blood pressure<140/90 mmHg (systolic/diastolic) at the Screening visit

Exclusion Criteria for Patients with IgAN
Individuals who meet any of the following exclusion criteria will not be eligible to participate:
1. Known or suspected allergy or hypersensitivity to any component of BION-1301, or history of severe hypersensitivity reaction to any monoclonal antibody
2. Donated blood in the 3 months prior to the first dose of study drug; plasma in the 7 days prior to the first dose of study drug; or platelets in the 6 weeks prior to the first dose of study drug
3. Poor venous access for the purpose of multiple blood draws and study drug administration as judged by the Investigator
4. Participated in any other study in which receipt of an investigational new drug, or investigational device occurred within 28 days, or 5 half-lives (whichever is longer) of first dose of study drug in the present study
5. Any confirmed or suspected immunosuppressive or immune-deficient state, including HIV infection or asplenia; recurrent, severe infections and chronic immunosuppressant medication (more than 28 days for inhaled/topical corticosteroids, and more than 1 week for systemic immunosuppressive or corticosteroid therapy) within 3 months prior to Day 1
6. Positive serology test for anti-HAV IgM, HBsAg, HCV antibodies (unless adequately treated for HCV), or antibodies to HIV-1 and/or HIV-2 at Screening
7. Major surgery or significant traumatic injury occurring within 28 days prior to first dose of study drug. If major surgery occurred >28 days prior to first dose of study drug, individual must have recovered adequately from the toxicity and/or complications from the intervention prior to the first dose of study drug
8. History or evidence of a clinically significant disorder, condition, disease or laboratory finding that could pose a risk to subject safety or interfere with the study, or would make the subject unsuitable for participation, as judged by the Investigator
9. Prior use of any approved biological agents, including monoclonal antibodies within 28 days or 5 half-lives (whichever is longer) prior to first dose of study drug in the present study
10. Secondary forms of IgAN as defined by the treating physician (e.g. IgA vasculitis and those with associated alcoholic cirrhosis)
11. Clinical suspicion of rapidly progressive glomerulonephritis (RPGN).
12. Any renal presentation for which the Investigator considers immediate treatment is needed including need for dialysis treatment or any planned kidney transplant during the study
13. Received systemic corticosteroid therapy (>10 mg/day of prednisone or equivalent for at least 10 days) or any other form of immunosuppressive therapy within 3 months prior to the first dose of study drug
14. Type 1 or 2 diabetes
15. Uncontrolled cardiovascular disease as judged by the Investigator
16. Current malignancy or history of malignancy during the last 3 years; exceptions include
   Adequately treated basal cell carcinoma
   Squamous cell carcinoma of the skin, or in situ cervical cancer
   Low-risk prostate cancer (i.e. Gleason score<7 and prostate specific antigen<10 ng/mL)
17. Intercurrent illness (including an active infection) that is either life-threatening or of clinical significance such that it might limit compliance with study requirements, or in the Investigator's assessment would place the subject at an unacceptable risk for study participation
18. Positive QuantiFERON-TB Gold Plus test at Screening
19. Received a live vaccination within 3 months prior to Screening through 3 months after the last dose of study drug. Non-replicating viral vector vaccines are acceptable.
20. Female who is breastfeeding or who has a positive serum pregnancy test at Screening or a positive urine pregnancy test prior to dosing on Day 1

The pharmacologically active dose (PAD) of BION-1301 is defined as a single dose predicted to cause a 95% decrease in free serum APRIL concentrations for a maximum duration of 24 hours and a minimal decrease of immune globulin (approximately 6% for IgG, approximately 15% for IgA). The estimated PAD is 50 mg based on PK-PD modelling of data from single-dose and multiple-dose toxicology studies in cynomolgus monkeys. The model accounted for differences in APRIL concentrations between cynomolgus monkeys and humans and applied allometric scaling to translate rate constants (PK and PD) and volumes (PK) between species.

A safety factor of 5 was applied to the PAD to account for potential toxicities not yet seen in patients with multiple myeloma in the clinical study (ADU-CL-16), and to account for the fact that the risk benefit profile is different between HVs and patients. In Part 1 of the current study, the first cohort started at 10 mg and consisted of 4 HVs: 3 receiving BION-1301 and one receiving placebo (versus 6 receiving BION-1301, 2 receiving placebo in subsequent cohorts). The reduced number of HVs for SAD-HV-1 is based on the assumption of an expected minor pharmacological effect with regard to immune globulin concentration; therefore, this data has limited utility for the characterization of the dose-exposure-effect relationship of BION-1301.

In addition to the PAD-based considerations described above, the starting dose of 10 mg is 600- to 2383-fold lower than the no observed adverse effect level (NOAEL) of 100 mg/kg in cynomolgus monkeys. Thus, adequate safety factors exist to support this initial dose of BION-1301 in healthy adults.

TABLE 35
Safety Factors for the Starting Dose in Healthy Volunteers

| Phase 1 Starting | Dose Safety | AUC Safety | Cmax Safety |
|---|---|---|---|
| Dose ^{a} | Factor ^{b} | Factor ^{c} | Factor ^{d} |
| 10 mg (0.17 | 600 | 1429 | 2383 |
| mg/kg) | | | |

AUC = area under the concentration-time curve;
Cₘₐₓ = maximum serum concentration;
SF = safety factor
^{a} 10 mg dose in human is equivalent to 0.17 mg/kg, assuming 60 kg human.
^{b} SF_{Dose} = Dose_{cyno}/Doseₕᵤₘₐₙ
^{c} SF_{AUC} = AUC_{cyno}/AUCₕᵤₘₐₙ. Sex-combined AUC₀₋₁₆₈ cyno at 100 mg/kg on Day 29 of study was 24,000 mg * day/L. The observed AUC_{0-inf} in subjects with multiple myeloma who received 50 mg was 84 mg * day/L (582 nM · day); assuming linear PK, this would result in an AUC_{0-inf} of 16.8 mg * day/L at the dose of 10 mg.
^{d} SFCₘₐₓ = (C_{max-cyno})/(Cₘₐₓ₋ₕᵤₘₐₙ), where C_{max-cyno} at 100 mg/kg is based on the observed mean C_{max-obs} after 5 doses on Day 29. Sex-combined Cₘₐₓ in cyno on Day 29 is 5720 μg/mL. The observed Cₘₐₓ in human at 50 mg was 12 mg/L (85.0 nM); assuming linear PK, this would result in a Cₘₐₓ of 2.4 mg/L at the dose of 10 mg.

Parts 1 and 2 were conducted, and baseline demographics in Part 1 (SAD) and Part 2 (MAD) are shown in the following Table 36:

TABLE 36
Baseline demographics for Parts 1 and 2.

| Group | SAD anti-APRIL | | SAD placebo | | MAD anti-APRIL | | MAD placebo | |
|---|---|---|---|---|---|---|---|---|
| N | 27 | | 9 | | 18 | | 9 | |
| Mean Age | 36.66 | (8.38) | 35.0 | (8.39) | 35.4 | (9.01) | 36.55 | (7.85) |
| (years) (SD) | | | | | | | | |
| Male | 27 | (100%) | 9 | (100%) | 18 | (100%) | 9 | (100%) |
| Female | 0 | | 0 | | 0 | | 0 | |
| Asian | 2 | (7.4%) | 3 | (33.3%) | 0 | | 0 | |
| Black/African | 6 | (22.2%) | 1 | (11.1%) | 3 | (16.7%) | 2 | (22.2%) |
| American | | | | | | | | |
| White | 18 | (66.7%) | 4 | (44.4%) | 11 | (61.1%) | 4 | (44.4%) |
| Multiple | 1 | (16.7%) | 1 | (11.1%) | 4 | (22.2%) | 3 | (33.3%) |
| Mean BMI | 25.46 | (2.47) | 23.73 | (2.94) | 25.65 | (3.00) | 25.95 | (1.56) |
| (kg/m2) (SD) | | | | | | | | |

BION-1301 was well tolerated in HVs. No SAEs, treatment discontinuations or events meeting stopping criteria were reported. All patients received pre-medication prior to first infusion, and 1 infusion related reaction was reported in the MAD 150 mg cohort. The most common AEs occurring in ≥10% of subjects in the MAD cohorts were headache, pain in extremity, elevated AST and nasopharyngitis. The most common AE occurring in ≥10% of subjects in the SAD cohorts was nasopharyngitis. Dosing was associated with a low incidence of anti-drug antibodies and neutralizing antibodies.

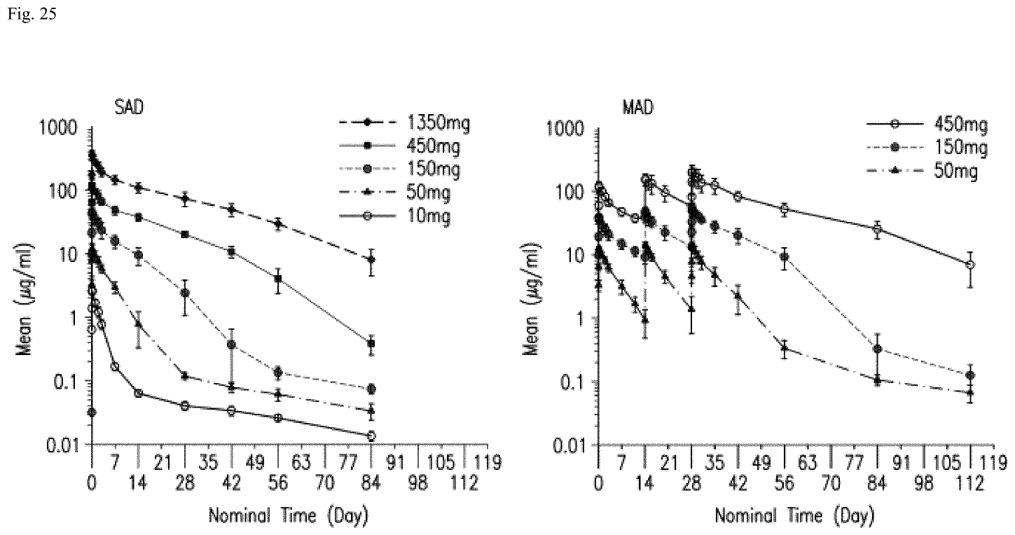

FIG. 25 shows mean BION-1301 serum concentrations (+/−SD) vs nominal time at the indicated dosing. Concentrations were similar within cohorts, with individual differences likely the result of fixed dose and variable body weights affecting drug disposition. Mean BION-1301 serum concentration was generally dose-proportional at low doses, but moderately greater than dose-proportional at higher doses.

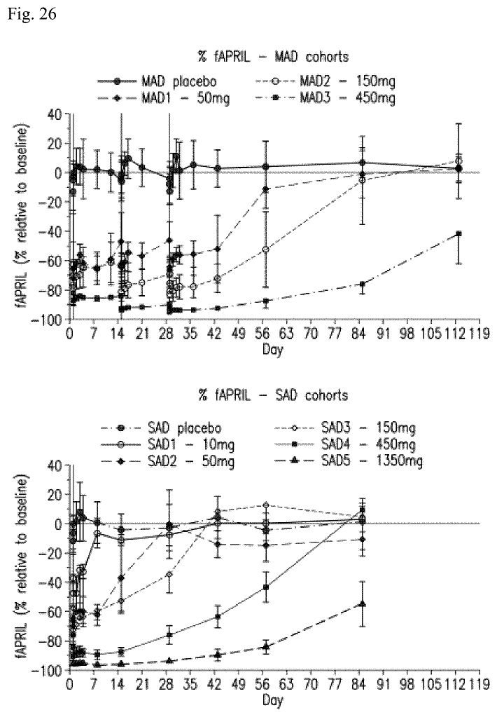

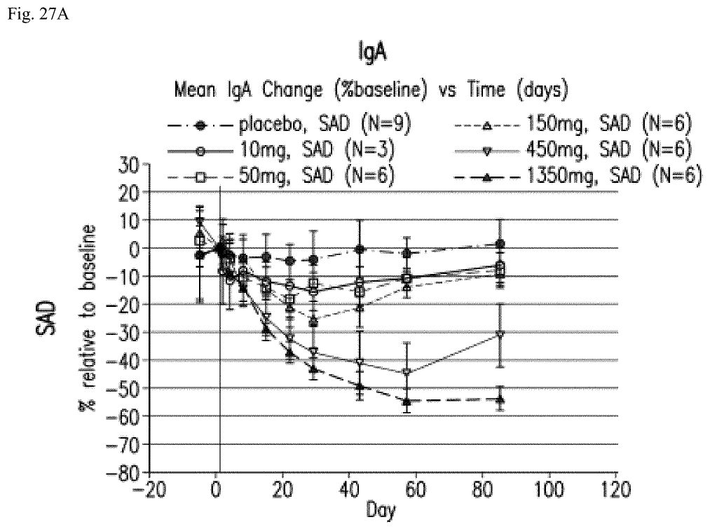

FIG. 26 shows mean free APRIL (fAPRIL) serum concentrations+/−SD vs nominal time at the indicated dosing. As shown, BION-1301 demonstrates a durable dose-dependent increase in target occupancy that is sustained for greater than one month at higher doses.

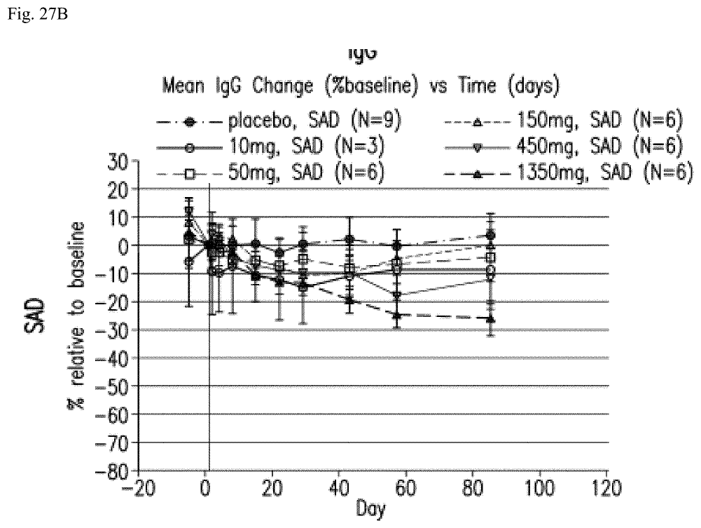

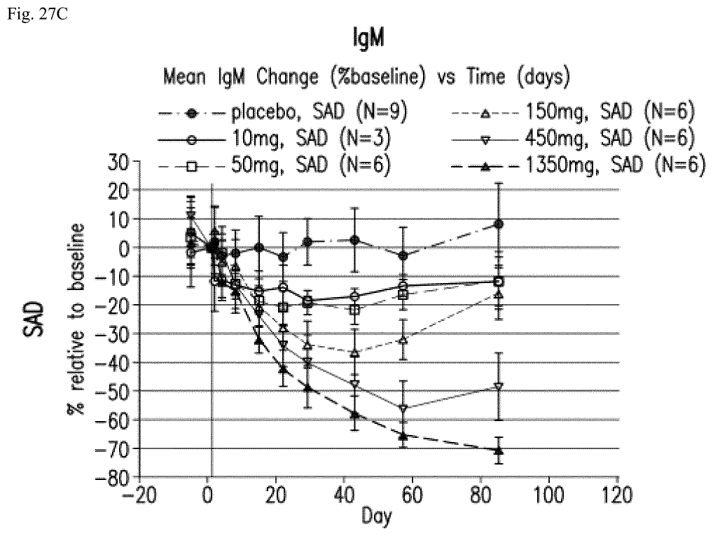

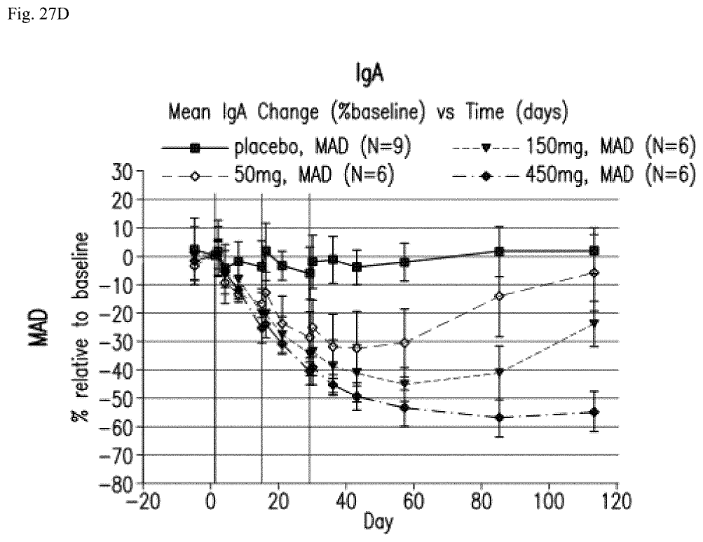

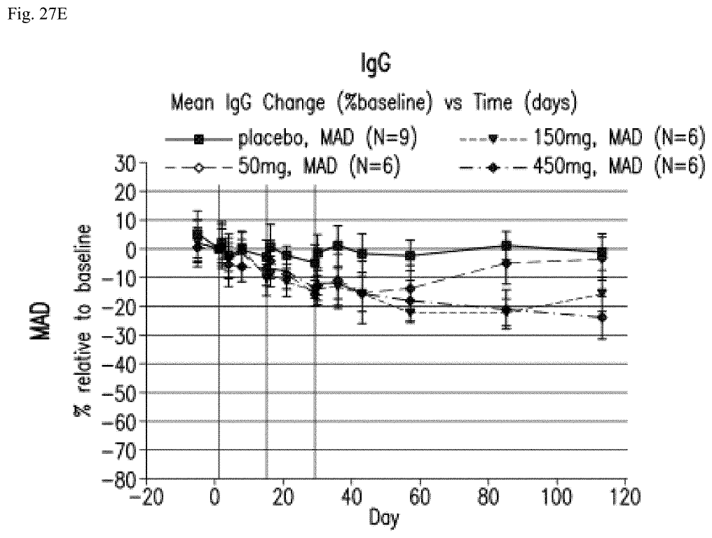

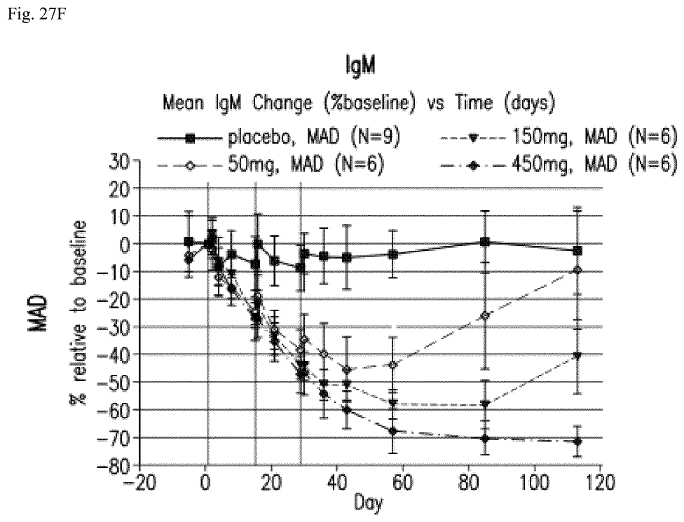

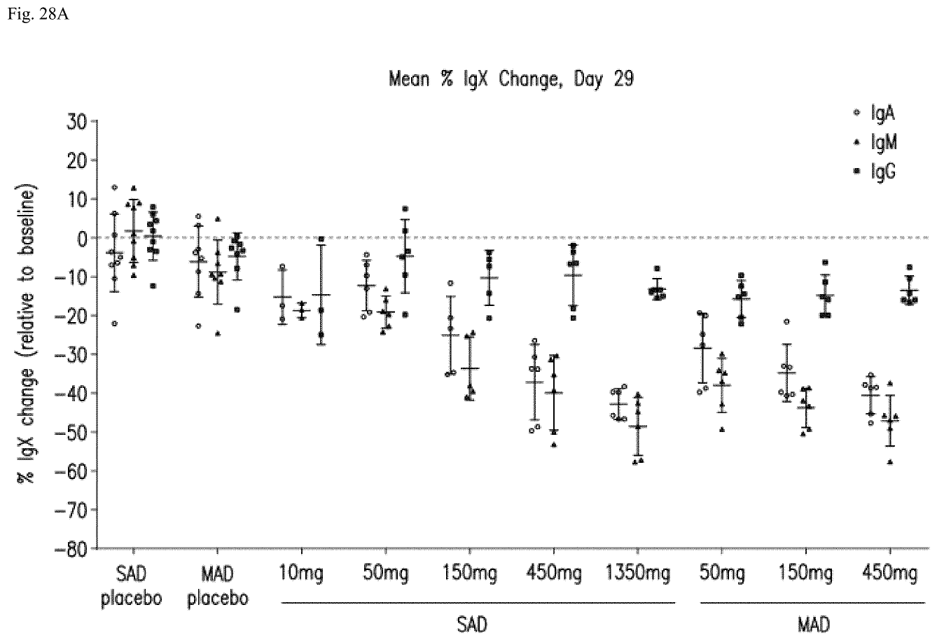

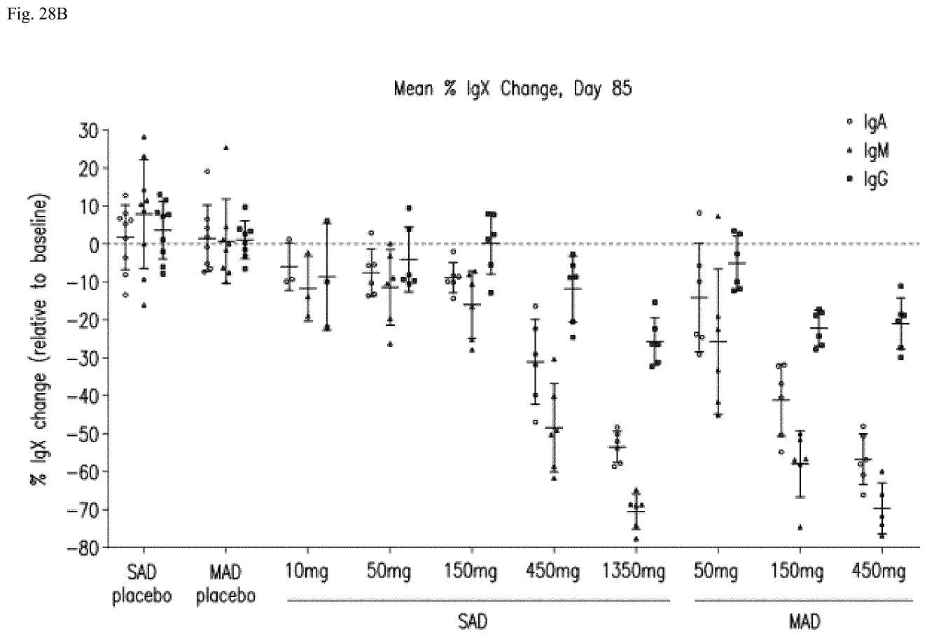

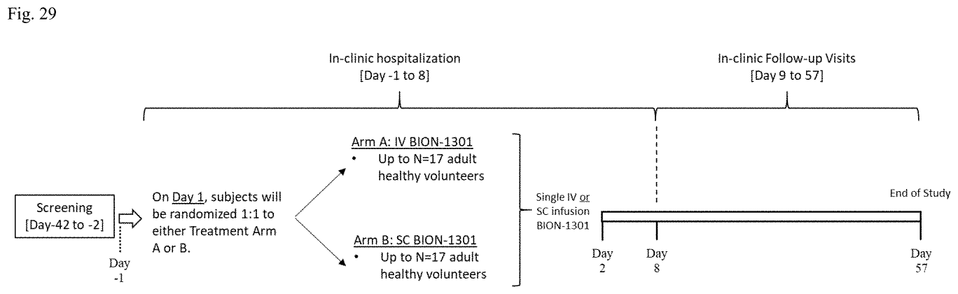

FIG. 27 shows the mean percent change (+/−SD) of immunoglobulin levels in serum relative to a baseline sample taken on Day 1 pre-dose. Panels A-C show single dose cohorts and panels D-F show multiple dose cohorts relative to baseline over time (days). BION-1301 dose dependently and durably reduces IgA and IgM, and to a lesser extent IgG. This data is consistent with a potential for monthly dosing of patients. At the 1350 mg single or 450 mg multiple dose levels, BION-1301 suppressed IgM levels into a low laboratory value range, however there were no reports of infection associated with treatment. BION-1301-mediated immunoglobulin reduction has the potential to disrupt the stoichiometry of IgA:IgG immune complexes. As show in in FIGS. 28A and B, BION-1301 provides a pharmacodynamic window to exploit IgA reduction while tempering impact to IgG.

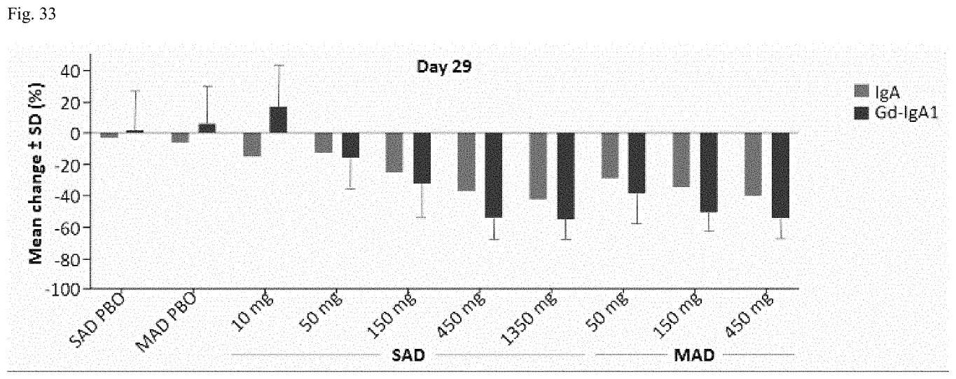

FIG. 33 shows reductions in serum IgA and Gd-IgA1 in a single ascending dose (SAD) and multiple ascending dose (MAD) study of BION-1301 administered by intravenous (IV) infusion in healthy human volunteers. As shown, BION-1301 provided a dose-dependent proportional reduction in both serum IgA and Gd-IgA1 levels.

From preliminary data from Part 3, 5 patients have been dosed Q2W with 450 mg BION-1301 by IV infusion for 12 weeks. Treatment was well tolerated, with no early terminations due to adverse events of SAEs. Rapid and sustained free APRIL reduction was observed, with durable reductions in Gd-IgA1, IgA and IgM, and smaller reductions in IgG observed. Clinically meaningful reductions in proteinuria (24-hr UPCR) was observed. The preliminary data shows early proof-of-concept for the disease modifying potential of BION-1301 to deplete pathogenic Gd-IgA1 and reduce proteinuria in patients with IgA nephropathy that remain at risk for progression with residual proteinuria despite optimized SOC treatment.

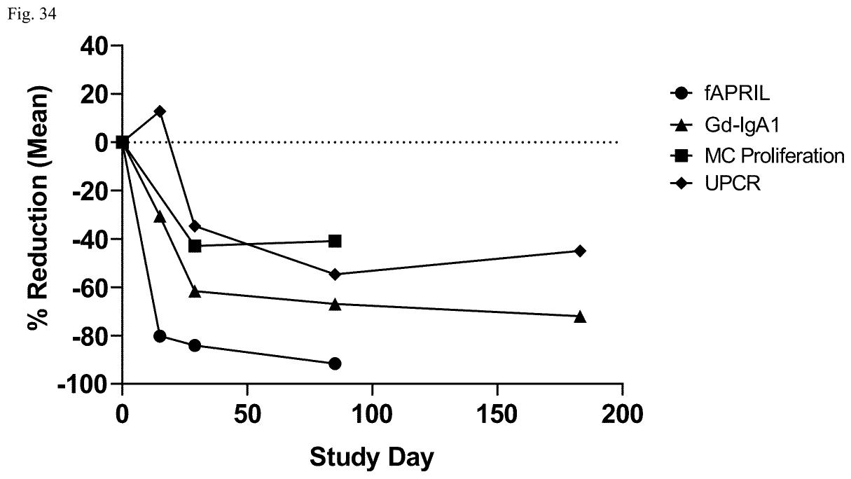

As a surrogate for the presence of pathogenic IgA-containing immune complexes in circulation, an ex vivo mesangial cell activation assay was used as a bioassay. IgA fractions were isolated by column chromatography from the plasma of the first two IgAN patients enrolled in the study at baseline, prior to BION-1301 administration and on Days 29 and 85 of BION-1301 treatment. Primary human mesangial cells in culture were then stimulated with these patient derived IgA containing fractions for 72 hours and mesangial cell proliferation was measured, in triplicate, by Brdu incorporation. As show in in FIG. 34, BION-1301 resulted in rapid APRIL neutralization, followed by Gd-IgA1 depletion and reduced mesangial cell activation, and subsequently proteinuria reduction in these IgAN patients.

### Example 16

The following was a Phase 1 single dose, parallel group safety and bioavailability study of BION-1301 administered intravenously (IV), or subcutaneously (SC) to adult healthy volunteers. This study was being conducted to define the bioavailability of BION-1301 when administered as a SC injection in order to assist in defining the recommended Phase 2 dose (RP2D) that will enable SC chronic dosing for the proposed indication of treatment of IgAN.

| | |
|---|---|
| Dosage Formulation | Sterile solution for parenteral administration |
| Appearance | Liquid in glass vials |
| Strength/ | For IV administration: 20 mg/mL in 10 mM |
| Concentration | L-histidine, 75 mM L-arginine, 3% sorbitol, |
| | and 0.01% (w/w) polysorbate 20, pH 6.3. |
| | (diluted with 0.9% normal saline prior to |
| | administration) |
| | For SC administration: 150 mg/mL in 10 mM |
| | L-histidine, 75 m L-arginine, 3% sorbitol, and |
| | 0.01% (w/w) polysorbate 20, pH 6.3. |
| Dosage Level(s) | 300 mg given as a single dose |
| Route of | IV infusion or SC injection |
| Administration | |

| | |
|---|---|
| Objectives | Endpoints |

Primary

| | |
|---|---|
| Estimate the bioavailability of | Subcutaneous bioavailability of |
| BION-1301 administered as a | BION-1301, calculated based on |
| single dose by the SC route | relative AUC_{0-inf} and AUC₀₋ₜ |
| compared to the IV route in | |
| adult healthy volunteers | |

Secondary

| | |
|---|---|
| Assess the safety and tolerability | Incidence and severity of treatment |
| of BION-1301 in adult healthy | emergent adverse events (TEAEs), |
| volunteers | serious adverse events (SAEs), and |
| | changes from baseline in safety |
| | parameters |
| Describe the pharmacokinetics | Pharmacokinetics (PK) of BION-1301 |
| (PK) of BION-1301 administered | when administered by the IV and SC |
| as a single dose of 300 mg by | routes, as assessed by C₀, Cₘₐₓ, |
| the IV and SC routes in adult | Tₘₐₓ, AUC_{0-inf}, AUC₀₋ₜ, Cₘᵢₙ, t½, |
| healthy volunteers | CL, and Vss. |
| Assess the pharmacodynamics (PD) | Pharmacodynamics (PD) of BION- |
| of BION-1301 administered as | 1301 when administered by the |
| a single dose of 300 mg by the IV | IV and SC routes, as assessed by |
| and SC routes in adult | changes in IgM, IgG, and IgA |
| healthy volunteers | |
| Assess the immunogenicity of | Incidence of anti-drug antibodies to |
| BION-1301 administered as a | BION-1301 when administered by |
| single dose of 300 mg by | the IV and SC routes |
| the IV and SC routes in | |
| adult healthy volunteers | |

Tertiary/Exploratory

| | |
|---|---|
| Characterize select biomarkers of | Changes from baseline in: |
| BION-1301 activity | Free a proliferation-inducing ligand |
| | (APRIL) levels |

The study was a Phase I, open label, randomized, single dose, parallel group safety and bioavailability study of BION-1301 administered by the IV and SC routes to adult healthy volunteers. The study enrolled up to approximately 34 subjects (17 per arm) to achieve a total of 30 PK evaluable subjects in a two-arm, parallel group, 3-period design. Subjects were randomized 1:1 to receive either BION-1301 by IV administration (Treatment Arm A) or BION-1301 by SC administration (Treatment Arm B). In both treatment arms, subjects will receive a single dose of BION-1301 of 300 mg. The study was conducted in 3 defined study periods: Screening Period, Treatment Period, and Safety Follow-up Period. Written informed consent for study participation was obtained before any study-related procedures or assessments were performed. Dosing (SC or IV) occurred in the clinic under the supervision of qualified personnel on Day 1. Subjects were closely monitored for the first 24 hours after dosing on Day 1 and during the in-clinic stay through Day 8. The study was complete once the last subject completes the final visit on Day 57. The study duration for any individual subject was up to 14 weeks and included a Screening Period of 6 weeks and Treatment (1 day) and Safety Follow-up Period of 8 weeks. Subjects received a single 300 mg dose of BION-1301 by 1 of 2 routes of administration, IV or SC. The IV dosing was prepared as described in Example 15, where a 20 mg/mL of antibody formulation was diluted into 0.9% saline, in this case 3×5 mL vials were used to provide 15 mL of antibody solution diluted into 235 mL of saline solution. The SC dosing was provided as a vial of 150 mg/mL formulation, and the desired 2.0 mL volume was removed with a syringe and injected directly into the abdomen.

Screening Period: Began when the informed consent form (ICF) was signed. During this period, the subject underwent assessments to determine eligibility for study participation. The Screening Period duration was up to 6 weeks. Subjects who met all eligibility criteria were enrolled into the study.

Admission/Treatment Period: Began on Day −1 with admission to the clinical facility and ended on Day 8 with discharge from the clinical facility. Dosing (SC or IV) occurred in the clinic under the supervision of qualified personnel on Day 1. Subjects were intensively monitored for the first 24 hours after dosing on Day 1 and then closely monitored until discharge on Day 8. During the Treatment Period, subjects underwent safety monitoring and assessment of PK and PD. Following completion of the final PK and PD sample collections and safety assessments on Day 8, subjects entered the Follow-up Period.

Follow-up Period: Began upon discharge from the clinical facility and ended on Day 57 with the End-of-Study (EOS) Visit as described in the SOE.

Participants eligible to be included in the study met all of the following criteria:
Participant must be 18-60 years of age at the time of signing the informed consent.
1) Body mass index (BMI) between 18-30 kg/m², inclusive, at Screening with a weight of ≥47 kg.
2) Non-smoker, defined as an individual who has not smoked previously and/or who has discontinued smoking or the use of nicotine/nicotine-containing products (including, but not limited to, snuff, e-cigarettes and similar products) at least 3 months before the Screening Visit, confirmed by medical history and negative cotinine level.
3) In good health, as determined by the investigator based on medical history, physical examination (PE), vital signs assessment, 12-lead ECG and clinical laboratory evaluations within normal reference ranges (or outside of normal reference ranges considered not clinically significant by the Investigator).
4) Is deemed by the investigator to be likely to comply with the protocol for the duration of the subject's participation in the study.
5) Is not currently taking prescription or over-the-counter medications, including dietary supplements, unless deemed suitable at the discretion of the investigator and the sponsor/medical monitor.
6) Participants can be male or female.
   Male participants were eligible to participate if they agreed to follow contraception guidance from Screening through 10 weeks after the last dose of study drug:
Be >3 months post-vasectomy
OR
Refrain from donating sperm from Screening through 10 weeks after the last dose of study drug
   PLUS either:
Be abstinent from heterosexual intercourse

### OR

Agree to use a male condom in conjunction with an additional highly effective female barrier contraception method when having sexual intercourse.
   Female participants were eligible to participate if they agreed to follow contraception guidance from Screening through 10 weeks after the last dose of study drug:
Is not a woman of childbearing potential (WOCBP) OR
Is a WOCBP and using a contraceptive method that was highly effective from Screening through 10 weeks after the last dose of study treatment and agrees not to donate eggs (ova, oocytes) for the purpose of reproduction during this period. If the highly effective method of contraception was hormonal, their male partner must also use a barrier method (i.e. a male condom). The investigator should evaluate the effectiveness of the contraceptive method in relationship to initiation of study treatment.
   A WOCBP must have a negative highly sensitive pregnancy test (serum as required by local regulations) within 24 hours before the dose of study treatment.
7) Capable of giving and understand signed informed consent and willing to participate in the study.

Participants were excluded from the study if any of the following criteria were met:
1) Regular consumption of alcohol within 6 months prior to Screening (>7 drinks/week for females, >14 drinks/week for males where 1 drink=5 ounces [150 mL] of wine or 12 ounces [360 mL] of beer or 1.5 ounces [45 mL] of hard liquor), or use of soft drugs (such as marijuana) within 3 months prior to the Screening Visit, or hard drugs (such as cocaine and phencyclidine) within 1 year prior to the Screening Visit and/or positive blood or urine test results for drugs of abuse or alcohol at the Screening Visit.
2) Donated blood in the 3 months prior to the single dose of study drug, plasma in the 7 days prior to the single dose of study drug, or platelets in the 6 weeks prior to the single dose of study drug, or received blood or blood products within 8 weeks of the single dose of study drug.
3) Used any prescription drugs, non-prescribed systemic or topical medication, remedy or supplement, any prescribed systemic or topical medication (with the exception of either paracetamol to a maximum of 2 g per day or ibuprofen up to 800 mg per day, hormone replacement therapy [HRT], or hormonal contraceptives) within 28 days or 5 half-lives (whichever was longer) of study drug administration unless deemed acceptable by the investigator and the medical monitor.
4) Poor venous access for the purposes of multiple blood draws and study drug administration, as judged by the Investigator.
5) Use of any approved biological agents, including monoclonal antibodies, within 28 days or 5 half-lives (whichever was longer) prior to the single dose of study drug in the present study.
6) Received a live vaccination within 3 months prior to Screening or planning to have a vaccination within 3 months after the dose of study drug.
7) Presence of any confirmed or suspected immunodeficient state OR recurrent, severe infections OR chronic systemic immunosuppressant medication use within 8 weeks prior to Day 1.
8) Positive serology test for the surface antigen of hepatitis B (HBsAg), hepatitis C (HCV) antibodies (unless adequately treated for HCV), or antibodies to (human immunodeficiency virus) HIV-1 and/or HIV-2.
9) History or evidence of a clinically significant disorder, condition, or disease that could pose a risk to subject safety or interfere with the study, or would make the subject unsuitable for participation, eg, clinically significant respiratory, renal, hepatic, gastrointestinal, hematological, lymphatic, neurological, cardiovascular, psychiatric disease.
10) Positive QuantiFERON-TB Gold Plus test at Screening.
11) Female who was breastfeeding or who has a positive serum pregnancy test at Screening or a positive serum pregnancy test on Day −1.
12) QTcF>450 msec for males and >470 msec for women or has clinically significant ECG findings as determined by the investigator.
13) Vital signs measures that include the following (one repeat measurement was allowed):
   a. Systolic blood pressure (BP)>140 mm/Hg or diastolic BP>90 mm/Hg.
   b. Systolic BP<90 mm/Hg or diastolic BP<50 mm/Hg.
   c. Pulse>100 beats per minute (bpm) or <40 bpm.
14) Participated in any other study with receipt of an investigational product or device within 28 days or 5 half-lives (whichever was longer) of dose of study drug in the present study.
15) Known or suspected allergy or hypersensitivity to any component of BION-1301, or history of severe hypersensitivity reaction to any monoclonal antibody.

For purposes of analysis, the following analysis populations are defined in Table 37.

TABLE 37
Analysis Populations.

| Population | Description |
|---|---|
| PK | All subjects who received a complete dose of study drug |
| | and for whom PK samples up to day 15 were obtained and |
| | successfully analyzed. |
| Safety | All subjects who received study |
| | treatment. |
| PD | All subjects who received study treatment and for whom |
| | at least one post-dose PD sample was obtained and |
| | successfully analyzed. |

A subject was considered enrolled once the subject passed all the inclusion and exclusion criterion and was been randomized to one of the treatment arms.

An evaluable subject was defined as an enrolled subject who received at least one dose of study treatment and provided post-baseline PK samples up to Day 15.

The statistical analysis plan (SAP) was developed and finalized before database lock and described in more detail the subject populations to be included in the analyses, and procedures for accounting for missing, unused, and spurious data.

All pharmacokinetic analyses were performed on the PK population using Phoenix® WinNonlin® (Version 8.1). Analyses for all PK endpoints were provided separately for each treatment arm. PK parameters were estimated by non-compartmental analysis, including but not limited to the following:

| Parameter | Definition |
|---|---|
| C₀ | Serum concentration extrapolated to time zero |
| Cₘₐₓ | Maximum observed serum concentration |
| Cₘᵢₙ | Minimum observed serum concentrations |
| tₘₐₓ | Time corresponding to occurrence of Cₘₐₓ |
| F | Bioavailability |
| AUCₗₐₛₜ | Area under the serum concentration-time curve |
| | from time 0 to last observed time point |
| AUC₀₋ₜ | Area under the serum concentration-time curve |
| | from time 0 to t |
| AUC_{0-inf} | Area under the serum concentration-time curve |
| | from time 0 to infinity Tₘₐₓ |
| | Time to maximum serum concentration |
| t_{1/2} | Terminal elimination half-life |
| CL | Clearance |
| Vₛₛ | Volume of distribution at steady state |
| MRT | Mean residence time |

Measurements and estimates for the PK parameters were tabulated and summarized by arm using descriptive statistics (mean, standard deviation [SD], coefficient of variation (CV %), median, minimum, and maximum). The PK summary statistics may include reporting of CV %, geometric mean, and geometric CV % as appropriate. Graphical presentations will include mean (±SD) serum concentration-time curves and individual subject concentration-time curves over the PK sampling times, when possible.

Specification of PK parameters for analysis; and statistical level of significance used, procedures for accounting for missing, unused data, procedures for reporting deviations, and selection of subjects to be included in the analysis populations were presented in the SAP as appropriate. A comparison of the PK parameters within each treatment arm were performed.

This study was completed, using BION-1301 20 mg/mL solution Lot No. P197673-0004L001/P02919 and 150 mg/mL solution Lot No. P217955-0001L001/P04819 from AMRI Glasgow. Subjects had samples collected for PK and PD, collected predose and at 0.25, 2, 4, 8 hours post end of infusion/injection, and 24 (day 2), 48 (day 3), 72 (day 4), 168 (day 8), 336 (dau 15), 672 (day 29), 1008 (day 43), and 1344 (day 57) hours post dose. Of the 17 subjects receiving a 300 mg IV dose by IV administration, two did not complete the study, while of the 17 subjects receiving a 300 mg dose by SC administration, three did not complete the study, and were considered lost to follow up. The 300 mg dose by either route was found to be safe and well-tolerated. The BION-1301 serum levels, free APRIL levels, and IgA, IgG and IgM antibody levels were measured.

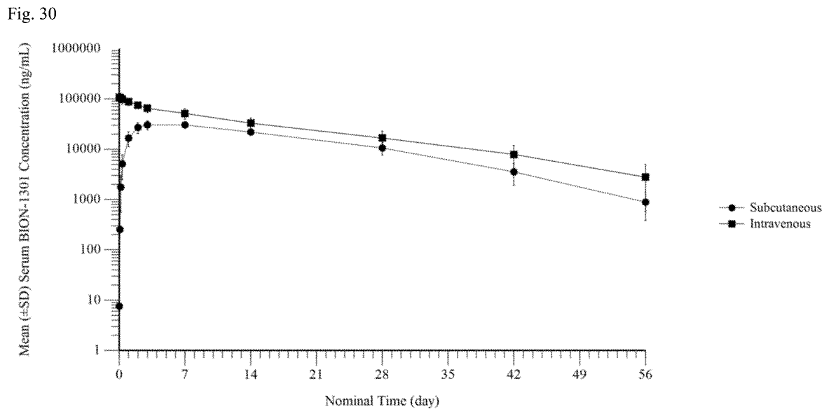

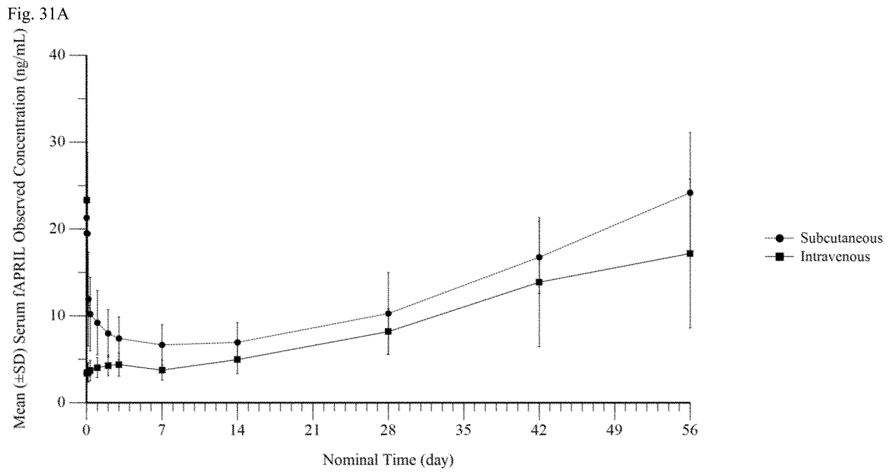

The mean (±SD) BION-1301 serum concentration levels are shown in FIG. 30, where time 0 is time of dosing BION-1301. The following Table 38 provides the pharmacokinetic parameters of BION-1301 for IV vs SC dose administration.

TABLE 38
Geometric mean summary BION-1301 PK parameters for IV vs SC dosing.

| Serum PK | | | | | |
|---|---|---|---|---|---|
| Parameter^{a} | Units | Intravenous | | Subcutaneous | |
| AUC_{0-14 d} | day * ng/mL | 750000 | (21.1); 16 | 357000 | (15.8); 16 |
| AUC_{0-28 d} | day * ng/mL | 1080000 | (23.5); 16 | 576000 | (16.1); 14 |
| AUC₀₋ₜ | day * ng/mL | 1280000 | (28.1); 16 | 639000 | (27.9); 16 |
| AUCₗₐₛₜ | day * ng/mL | 1330000 | (29.7); 16 | 703000 | (18.4); 14 |
| Cₘₐₓ | ng/mL | 109000 | (20.0); 17 | 31900 | (15.5); 17 |
| tₘₐₓ^{b} | h | 2.28 | (2.25, 4.05); 17 | 72.85 | (48.73, 168.02); 17 |
| t_{1/2} | day | 11.1 | (24.3); 16 | 7.63 | (24.2); 14 |
| MRT_{inf} | day | 16.3 | (19.6); 16 | 16.9 | (12.8); 14 |

| | | | | |
|---|---|---|---|---|
| CL | L/day | 0.225 | (29.7); 16 | NR |

| | | | | |
|---|---|---|---|---|
| CL/F | L/day | NR | 0.427 | (18.4); 14 |

| | | | | |
|---|---|---|---|---|
| Vₛₛ | L | 3.66 | (16.3); 16 | NR |

^{a}Geometric Mean (Geometric CV %); N
^{b}Median (Min; Max); N
NR = not reported

The relative bioavailability was evaluated between IV BION-1301 (reference) and SC BION-1301 (test) treatment using an ANOVA and the results are shown in the following Table 39, where Test treatment is 300 mg BION-1301 administered SC, Reference treatment is 300 mg BION-1301 administered intravenously, and CI=confidence interval, CV=coefficient of variation, LSM=least square means, and N=number of observations.

TABLE 39
Relative Bioavailability between Intravenous and Subcutaneous BION-1301 Treatments.

| | | Test | Reference | Ratio of | | Inter- |
|---|---|---|---|---|---|---|
| Serum PK | | Geometric | Geometric | Geometric | | Subject |
| Parameter | Units | LSM; N | LSM; N | LSM (%) | 90% CI | (CV %) |
| AUC_{inf} | day * ng/mL | 702500; 14 | 1334000; 16 | 52.67 | (45.18, 61.41) | 25.0 |
| AUC₀₋ₜ | day * ng/mL | 638900; 16 | 1282000; 16 | 49.84 | (42.27, 58.78) | 28.0 |
| Cₘₐₓ | ng/mL | 31940; 17 | 108500; 17 | 29.43 | (26.54, 32.63) | 17.9 |
| AUC_{0-14 d} | day * ng/mL | 357200; 16 | 750100; 16 | 47.62 | (42.62, 53.21) | 18.6 |
| AUC_{0-28 d} | day * ng/mL | 575800; 14 | 1075000; 16 | 53.54 | (47.22, 60.71) | 20.4 |

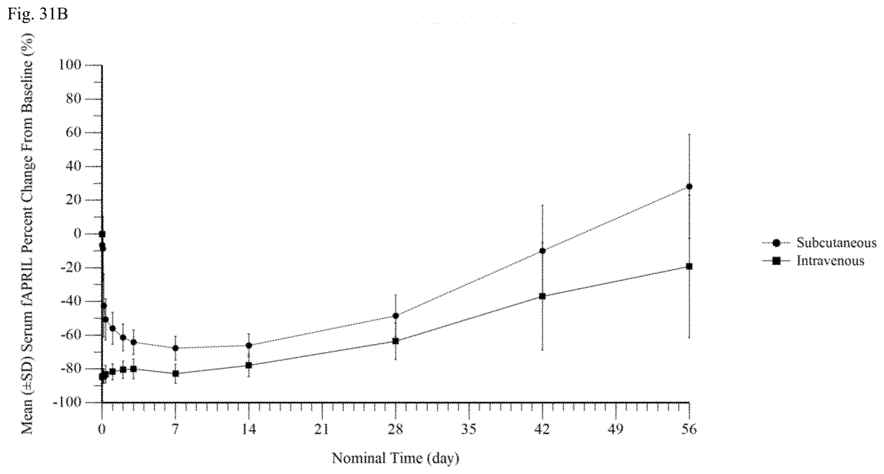

For the measurement of APRIL concentrations, the mean (±SD) fAPRIL concentrations after single-dose IV or SC administration are shown in FIG. 31A and the mean (±SD) percent change relative to the baseline of fAPRIL after single-dose IV or SC administration are shown in FIG. 31B, where time 0 is time of dosing BION-1301. The following Table 40 provides the pharmacodynamic parameters of fAPRIL after IV vs SC dose administration, where the AUEC Below B₀₋ₜ is the area of the response curve that is below the baseline effect value from time point 0 to time point t, using the linear trapezoid interpolation rule, Rₘᵢₙ is the minimum observed response value post-dose, PBRₘᵢₙ is the minimum percent change from baseline response value post-dose, calculated as (Rₘᵢₙ−B)/B×100, and tₘᵢₙ is the time of Rₘᵢₙ.

TABLE 40
Mean summary fAPRIL pharmacodynamic parameters.

| Serum PD Parameter^{a} | Units | Intravenous | | Subcutaneous | |
|---|---|---|---|---|---|
| AUEC_Below_B | day * ng/mL | 804 | (52.2); 16 | 446 | (58.1); 16 |
| AUEC Below B_{0-7 d} | day * ng/mL | 134 | (40.7); 17 | 93.3 | (47.9); 17 |
| AUEC Below B_{0-14 d} | day * ng/mL | 274 | (40.9); 16 | 195 | (49.3); 16 |
| AUEC Below B_{0-28 d} | day * ng/mL | 519 | (42.6); 16 | 345 | (45.3); 14 |
| Baseline | ng/mL | 23.3 | (36.4); 17 | 21.3 | (41.2); 17 |
| Rₘᵢₙ | ng/mL | 3.13 | (32.3); 17 | 6.29 | (35.5); 17 |
| PBRₘᵢₙ | % | −86.0 | (−4.3); 17 | −69.5 | (−10); 17 |
| tₘᵢₙ^{b} | h | 4.00 | (2.25, 168.05); 17 | 168.02 | (8.00, 673.83); 17 |

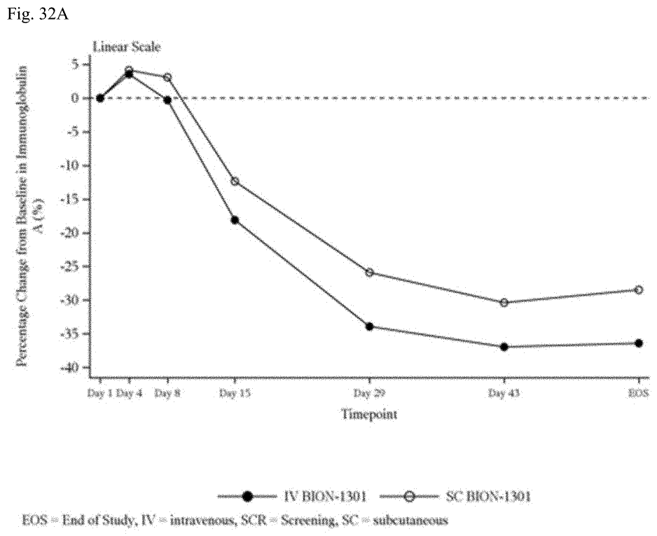

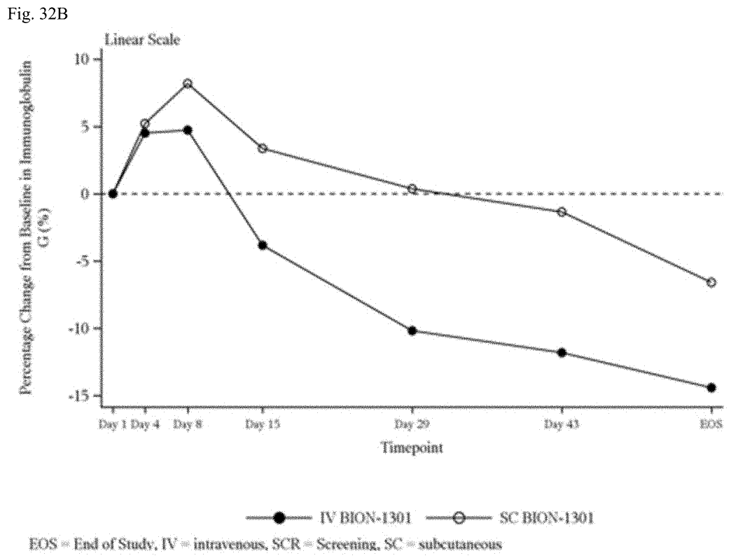

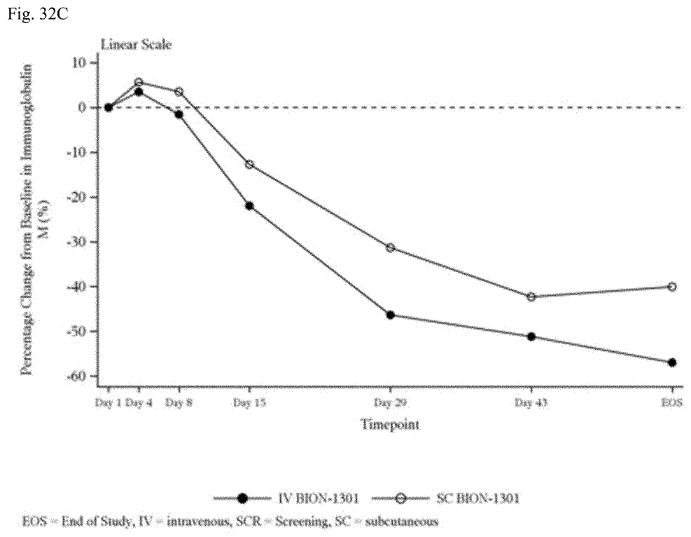

For the measurement of immunoglobulin levels, the mean % change relative to baseline in serum immunoglobulin levels over time for IV vs SC dosing of BION-1301 are shown in FIGS. 32A (IgA), 32B (IgG), and 32C (IgM).

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention.

It is to be understood that the invention is not limited in its application to the details of construction and to the arrangements of the components set forth in the following description or illustrated in the drawings. The invention is capable of embodiments in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

While the invention has been described and exemplified in sufficient detail for those skilled in this art to make and use it, various alternatives, modifications, and improvements should be apparent without departing from the spirit and scope of the invention. The examples provided herein are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patent applications, patents, publications and other references mentioned in the specification are indicative of the levels of those of ordinary skill in the art to which the invention pertains and are each incorporated herein by reference. The references cited herein are not admitted to be prior art to the claimed invention.

Unless otherwise defined, all technical and scientific terms used herein have the same meaning as commonly understood by one of ordinary skill in the art to which this invention belongs. In the case of conflict, the present specification, including definitions, will control.

The use of the articles “a”, “an”, and “the” in both the description and claims are to be construed to cover both the singular and the plural, unless otherwise indicated herein or clearly contradicted by context. The terms “comprising”, “having”, “being of” as in “being of a chemical formula”, “including”, and “containing” are to be construed as open terms (i.e., meaning “including but not limited to”) unless otherwise noted. Additionally whenever “comprising” or another open-ended term is used in an embodiment, it is to be understood that the same embodiment can be more narrowly claimed using the intermediate term “consisting essentially of” or the closed term “consisting of”.

The term “about”, “approximately”, or “approximate”, when used in connection with a numerical value, means that a collection or range of values is included. For example, “about X” includes a range of values that are ±20%, ±10%, ±5%, ±2%, ±1%, ±0.5%, ±0.2%, or ±0.1% of X, where X is a numerical value. In one embodiment, the term “about” refers to a range of values which are 10% more or less than the specified value. In another embodiment, the term “about” refers to a range of values which are 5% more or less than the specified value. In another embodiment, the term “about” refers to a range of values which are 1% more or less than the specified value.

Recitation of ranges of values are merely intended to serve as a shorthand method of referring individually to each separate value falling within the range, unless otherwise indicated herein, and each separate value is incorporated into the specification as if it were individually recited herein. A range used herein, unless otherwise specified, includes the two limits of the range. For example, the terms “between X and Y” and “range from X to Y, are inclusive of X and Y and the integers there between. On the other hand, when a series of individual values are referred to in the disclosure, any range including any of the two individual values as the two end points is also conceived in this disclosure. For example, the expression “a dose of about 100 mg, 200 mg, or 400 mg” can also mean “a dose ranging from 100 to 200 mg”, “a dose ranging from 200 to 400 mg”, or “a dose ranging from 100 to 400 mg”.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims.

We claim:

1. An antibody formulation suitable for pharmaceutical infusion or subcutaneous injection, comprising:
an anti-APRIL antibody at a concentration of between about 20 mg/mL to about 190 mg/mL;
about 10 mM L-histidine;
about 75 mM L-arginine;
about 3% wt % sorbitol;
about 0.01 wt % polysorbate 20; and
a pH of about 6.0 to about 6.6;
wherein the formulation (i) has a viscosity of about 16 cP or less, (ii) does not comprise glutamic acid or its salt, (iii) has an osmolality of between about 250 mOsm/kg to about 390 mOsm/kg, and (iv) has an OD330 of less than about 1.0.

2. An antibody formulation according to claim 1, wherein the formulation maintains at least 96% purity of the anti-APRIL antibody following storage at 2-8° C. for 9 months following manufacture of the formulation.

3. An antibody formulation according to claim 2, wherein the formulation maintains at least 95% purity of the anti-APRIL antibody following storage at 25° C. for 6 months following manufacture of the formulation.

4. An antibody formulation according to claim 1, wherein the formulation has a second virial coefficient of 2.5×10⁻⁵ mol·mL/g² or greater measured at 25° C.

5. An antibody formulation according to claim 1, wherein the formulation has a calculated isoelectric point of about 7.4 or greater.

6. An antibody formulation according to claim 1, wherein the anti-APRIL antibody in the formulation is at a concentration of about 150 mg/mL.

7. An antibody formulation according to claim 6, wherein the formulation has an osmolality of between about 290 mOsm/kg to about 390 mOsm/kg.

8. An antibody formulation according to claim 6, wherein the formulation has an OD330 of about 0.8 or less.

9. An antibody formulation according to claim 1, wherein the anti-APRIL antibody is at a concentration of about 20 mg/mL.

10. An antibody formulation according to claim 9, wherein the formulation has an osmolality of between about 293 mOsm/kg to about 333 mOsm/kg.

11. An antibody formulation according to claim 1, wherein the anti-APRIL antibody is a humanized antibody comprising a heavy chain variable region.light chain variable region pair selected from the group consisting of VH11.VL15, VH12.VL15, VH13.VL15, VH14.VL15, VH14_1.VL15, VH14_1C.VL15, VH14_1D.VL15, VH14_1E.VL15, and VH14_1G.VL15.

12. An antibody formulation according to claim 1, wherein the formulation is free of glycine, carbonate, HEPES, phosphate, citrate, and acetate.

13. A single-use or multi-use vial comprising the antibody formulation of claim 1.

14. The single-use or multi-use vial of claim 13 containing a volume of between 0.5 mL and 50 mL of the formulation having an anti-APRIL antibody concentration of between about 20 mg/mL to about 190 mg/mL.

15. The single-use or multi-use vial of claim 14, wherein the formulation has an anti-APRIL antibody concentration of about 20 mg/mL.

16. The single-use or multi-use vial of claim 14, wherein the formulation has an anti-APRIL antibody concentration of about 150 mg/mL.

17. The single-use or multi-use vial of claim 14, containing a volume of 5 mL of the formulation.

18. A pre-filled syringe, autoinjector, or injector pen comprising the antibody formulation of claim 1.

19. The pre-filled syringe, autoinjector, or injector pen of claim 18 containing a volume of between about 0.5 mL and about 10 mL of the formulation having an anti-APRIL antibody concentration of between about 20 mg/mL to about 190 mg/mL.

20. The pre-filled syringe, autoinjector, or injector pen of claim 19, wherein the formulation has an anti-APRIL antibody concentration of about 20 mg/mL.

21. The pre-filled syringe, autoinjector, or injector pen of claim 19, wherein the formulation has an anti-APRIL antibody concentration of about 150 mg/mL.

22. The pre-filled syringe, autoinjector, or injector pen of claim 19, containing a volume of about 2 mL of the formulation.

23. A method of administering an anti-APRIL antibody to an individual in need thereof comprising administering the formulation of claim 1 by subcutaneous injection into the individual.

24. A method according to claim 23, wherein the method comprises repeating the administration on at least an every week (QW) schedule for at least 2 administration cycles.

25. A method according to claim 23, wherein the method comprises repeating the administration on at least an every two weeks (Q2W) schedule for at least 2 administration cycles.

26. A method according to claim 23, wherein the method comprises repeating the administration on at least an every 4 weeks (Q4W) or monthly (QMT) schedule for at least 2 administration cycles.

27. A method according to claim 23, wherein a total dose of between about 10 mg to about 1350 mg of the anti-APRIL antibody is administered per dosing event.

28. A method according to claim 27, wherein about 2 mL of the formulation at a concentration of about 150 mg/mL of the anti-APRIL antibody is delivered per administration, and each dosing event comprises one or more of said administrations.

29. A method according to claim 27, wherein about 4 mL of the formulation at a concentration of about 150 mg/mL of the anti-APRIL antibody is delivered per administration, and each dosing event comprises one or more of said administrations.

30. A method according to claim 23, wherein the formulation is subcutaneously administered into a site in the individual's thigh, abdomen, or upper arm.

31. A method of administering an anti-APRIL antibody to an individual in need thereof comprising administering the formulation of claim 1 by intravenous infusion into the individual.

32. A method according to claim 31, wherein the method comprises repeating the administration on at least a QW schedule for at least 2 administration cycles.

33. A method according to claim 31, wherein the method comprises repeating the administration on at least a Q2W schedule for at least 2 administration cycles.

34. A method according to claim 31, wherein the method comprises repeating the administration on at least a Q4W or monthly schedule for at least 2 administration cycles.

35. A method according to any claim 31, wherein the intravenous infusion comprises:
diluting the formulation of claim 1 to a concentration of between about 0.1 mg/mL to about 10 mg/mL in 0.9% saline; and
administering a total dose of between about 10 mg to about 1350 mg of the anti-APRIL antibody to the individual in a single intravenous dose of the diluted formulation over a period of about 2 hours.

36. A method according to claim 35, wherein 15 mL of the formulation at a concentration of 20 mg/mL is added to 235 mL of 0.9% saline to provide the intraveneous dose at a concentration of 1.2 mg/mL.

37. A method of administering an anti-APRIL antibody to an individual in need thereof comprising administering an anti-APRIL by a loading/maintenance administration protocol.

38. A method according to claim 37, wherein the loading component of the loading/maintenance administration protocol comprises one or more administrations of the anti-APRIL antibody at a higher concentration than the anti-APRIL antibody concentration in the maintenance component of the loading/maintenance administration protocol.

39. A method according to claim 37, wherein the loading component of the loading/maintenance administration protocol comprises one or more administrations of the anti-APRIL antibody at a higher frequency than the frequency of administration of the anti-APRIL antibody in the maintenance component of the loading/maintenance administration protocol.

40. A method according to claim 37, wherein the loading component of the loading/maintenance administration protocol comprises one or more administrations of the anti-APRIL antibody at a different route than the route of administration of the anti-APRIL antibody in the maintenance component of the loading/maintenance administration protocol.

41. A method according to claim 37, wherein the loading component of the loading/maintenance administration protocol comprises one or more intravenous administrations of the anti-APRIL antibody and the maintenance component of the loading/maintenance administration protocol comprises one or more subcutaneous administrations of the anti-APRIL antibody.

42. A method according to claim 37, wherein the anti-APRIL antibody is an antibody formulation according to claim 1.

43. A method according to claim 23, wherein the individual in need thereof has a serum IgA level greater than 4 g/L.

44. A method according to claim 23, wherein the individual in need thereof is an IgA nephropathy patient.

45. A method according to claim 23, wherein the individual has a hyperimmunoglobulinemia.
